(19) United States
(12) Patent Application Publication
(10) Pub. No.: US 2022/0235405 A1
(43) Pub. Date: Jul. 28, 2022

(54) METHODS AND RELATED KITS FOR SPATIAL ANALYSIS

(71) Applicant: Encodia, Inc., San Diego, CA (US)

(72) Inventors: Mark S. CHEE, San Diego, CA (US); Kevin L. GUNDERSON, San Diego, CA (US)

(73) Assignee: Encodia, Inc., San Diego, CA (US)

(21) Appl. No.: 17/611,523

(22) PCT Filed: May 19, 2020
(86) PCT No.: PCT/US2020/033658

§ 371 Date: Nov. 15, 2021

Related U.S. Application Data
(60) Provisional application No. 62/850,426, filed on May 20, 2019.
(60) Provisional application No. 62/850,410, filed on May 20, 2019.

(51) Int. Cl. C12Q 1/6841

(52) U.S. Cl. CPC C12Q 1/6841

(57) ABSTRACT

Provided herein are methods and compositions for spatial analysis of macromolecules (e.g., proteins, polypeptides, or peptides). In some embodiments, the methods are for analyzing a macromolecule or a plurality of macromolecules, (e.g., peptides, polypeptides, and proteins) including determining spatial information and sequencing the macromolecule. In some embodiments, the analysis employs barcoding and/or nucleic acid encoding of molecular recognition events, and/or detectable labels. Also provided are compositions, e.g., kits, containing components for performing the provided methods for analysis of the macromolecule.

### RELATED APPLICATIONS

The present application claims priority to U.S. provisional patent application No. 62/850,410, filed on May 20, 2019 and U.S. provisional patent application No. 62/850,426, filed on May 20, 2019, the disclosures and contents of each are incorporated by reference in their entireties for all purposes.

### SEQUENCE LISTING ON ASCII TEXT

This patent or application file contains a Sequence Listing submitted in computer readable ASCII text format (file name: 4614-2001840_20200518 SeqList_ST25.txt, recorded: 18 May 2020, size: 693 bytes). The content of the Sequence Listing file is incorporated herein by reference in its entirety.

### TECHNICAL FIELD

The present disclosure relates to methods and compositions for analysis or spatial analysis of macromolecules (e.g., proteins, polypeptides, or peptides). In some embodiments, the methods are for analyzing a macromolecule or a plurality of macromolecules, (e.g., peptides, polypeptides, and proteins) including assessing or determining spatial information, characteristics, sequence, and/or identity of the macromolecule(s). In some embodiments, the analysis employs barcoding and nucleic acid encoding of molecular recognition events, and/or detectable labels. Also provided are compositions, e.g., kits, containing components for performing the provided methods for analysis of the macromolecule(s).

### BACKGROUND

Existing methods for identifying and analyzing molecules from a sample while providing information regarding characteristics of the sample, for example, the identity, concentration and/or spatial distribution of multiple macromolecules in a sample are limited. For example, known approaches for identifying proteins while retaining other sample or spatial information is not appropriate for analyzing a large number of unknown proteins within a sample. Some current techniques may detect only a few targets at one time and require use of additional biological samples from a source which limits the ability to determine relative characteristics of the targets between samples. Moreover, in certain instances, a limited amount of sample may be available for analysis or the individual sample may require further analysis, including analysis of the identity and/or sequence of the proteins. In some cases, imaging based approaches for large numbers of cells may lack the ability to provide information regarding the cellular features of the sample, such as cell types or phenotypes. Accordingly, there remains a need in the art for improved techniques relating to macromolecule (e.g., polypeptide or polynucleotide) analysis that is multiplex and/or also allows characterization which can provide spatial information, identity, and/or sequencing of proteins that is highly-parallelized, accurate, sensitive, and/or high-throughput.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided herein is a method for analyzing a macromolecule including: providing a spatial sample comprising a macromolecule associated with a recording tag at a spatial location; assessing (e.g., observing) the spatial location of the macromolecule in the spatial sample in situ; binding a molecular probe comprising a probe tag to the macromolecule or a moiety in proximity to the macromolecule in the spatial sample; extending the recording tag by transferring information from the probe tag in the molecular probe to the recording tag, wherein transferring information from the probe tag to the recording tag generates an extended recording tag; determining at least the sequence of the probe tag in the extended recording tag; and correlating the sequence of the probe tag in the extended recording tag with the molecular probe and/or spatial location assessed, thereby associating information from the sequence of the extended recording tag or a portion thereof with the observed spatial location of the macromolecule.

Provided herein are methods of analyzing a macromolecule (e.g., protein, polypeptide, or peptide) comprising steps: (a) providing a spatial sample comprising a macromolecule associated with a recording tag; (b1) providing a spatial probe comprising a spatial tag to the spatial sample; (b2) assessing the spatial tag in situ to obtain the spatial location of the spatial tag in the spatial sample; (b3) extending the recording tag by transferring information from the spatial tag in the spatial probe to the recording tag; (c1) binding a molecular probe comprising a probe tag to the macromolecule or a moiety in proximity to the macromolecule in the spatial sample; (c2) extending the recording tag by transferring information from the probe tag in the molecular probe to the recording tag, wherein transferring information from the spatial tag and/or probe tag to the recording tag generates an extended recording tag; (d) determining at least the sequence of the probe tag and spatial tag in the extended recording tag; and (e) correlating the sequence of the spatial tag determined in step (d) with the spatial tag assessed in step (b2); thereby associating information from the sequence of the extended recording tag or a portion thereof, e.g., the information from the spatial tag and/or probe tag, determined in step (d) with the spatial location of the spatial probe assessed in step (b2).

In some embodiments, the method is for analyzing a plurality of macromolecules. In some aspects, the macromolecule is a polypeptide. In some cases, the method further includes performing a macromolecule analysis assay or a polypeptide analysis assay. In some embodiments, the method includes binding a plurality of molecular probes and plurality of spatial probes to the spatial sample. In some embodiments, information from more than one probe tag is transferred to a recording tag. In some embodiments, information from more than one spatial tag is transferred to a recording tag. In some embodiments, cycles of binding with molecular probes, transferring information from the probe tags associated with the molecular probes to the recording tag (thereby extending the recording tag and generating an extended recording tag), binding with the spatial probes, and transferring information from the spatial tags associated with the spatial probes to the recording tag (thereby extending the recording tag and generating an extended recording tag) is performed. The probe tags and/or the spatial tags may include a barcode, in addition other optional nucleic acid components. In some embodiments, one or more of the provided steps are repeated one or more times. In some aspects, the order of performing at least some of the steps of the method may be altered.

In some embodiments, the method further includes performing a macromolecule analysis assay, such as a polypeptide analysis assay. The macromolecule analysis assay includes contacting the macromolecule with a one or more binding agents and transferring identifying information from a coding tag associated with the binding agent to the recording tag. In some embodiments, the contacting of the macromolecule with the binding agent and transferring information from the coding tag to the recording tag is repeated two or more times. In some embodiments, the macromolecules and associated recording tags comprising information transferred from the probe tag and spatial tag are released from the spatial sample prior to performing the macromolecule analysis assay. In some of any such embodiments, the macromolecule analysis assay includes one or more cycles of contacting the macromolecule with a binding agent capable of binding to the macromolecule, wherein the binding agent comprises a coding tag with identifying information regarding the binding agent; and transferring the information of the coding tag to the recording tag to further extend the extended recording tag. In some embodiments, the extended recording tag comprises information from one or more spatial tags, one or more probe tags, and optionally one or more coding tags.

Provided herein are methods of analyzing a macromolecule (e.g., protein, polypeptide, or peptide) comprising steps: (a) providing a spatial sample comprising a macromolecule with a recording tag; (b) binding a molecular probe comprising a detectable label and a probe tag to the macromolecule or a moiety in proximity to the macromolecule in the spatial sample; (c) transferring information from the probe tag in the molecular probe to the recording tag to generate an extended recording tag; (d) assessing, e.g., observing, the detectable label to obtain spatial information of the molecular probe; (e) determining at least the sequence of the probe tag in the extended recording tag; and (f) correlating the sequence of the probe tag determined in step (e) with the molecular probe; thereby associating information from the sequence determined in step (e) with its spatial information determined in step (d). In some embodiments, the method is for analyzing a plurality of macromolecules. In some aspects, the macromolecule is a polypeptide. In some embodiments, the method includes binding a plurality of molecular probes each comprising a detectable label and a probe tag to the spatial sample. The molecular probe may bind to a macromolecule in the spatial sample or a moiety in proximity to the macromolecule in the spatial sample. In some embodiments, the molecular probe binds to a moiety that is bound to, associated with or complexed with the macromolecule in the spatial sample. In some embodiments, information from more than one probe tag is transferred to a recording tag. In some embodiments, cycles of binding with molecular probes, transferring information from the molecular probe to the recording tag, and/or assessing, e.g., observing, the detectable label are performed. In some aspects, the order of performing at least some of the steps of any of the provided methods may be altered. In some embodiments, the recording tags are not associated with or attached to the macromolecule. In some embodiments, the recording tags are associated with or attached to the macromolecule.

In some embodiments, the method further includes performing a macromolecule analysis assay. In some cases, the macromolecule analysis assay is a polypeptide analysis assay which comprises contacting the macromolecule with a binding agent associated with a coding tag and transferring information from the coding tag to the recording tag, thereby extending the recording tag. In some embodiments, the macromolecules and associated recording tags comprising information transferred from the probe tag is released from the spatial sample prior to performing the macromolecule analysis assay. In some of any such embodiments, the macromolecule analysis assay includes one or more cycles of contacting the macromolecule with a binding agent capable of binding to the macromolecule, wherein the binding agent comprises a coding tag with identifying information regarding the binding agent; and transferring the information of the coding tag to the recording tag to further extend the extended recording tag.

Also provided herein are kits and reagents for performing any of the methods for analyzing macromolecule, e.g., polypeptides, provided herein. In some embodiments, the kits comprise one or more of the following components: spatial probe(s), spatial tag(s), molecular probe(s), probe tag(s), reagent(s) for sequencing, reagent(s) for performing nucleic acid extension recoding tag(s), reagent(s) for attaching or transferring the recording tag, binding agent(s), reagent(s) for transferring identifying information from the probe tag or spatial tag to the recording tag, reagent(s) for transferring identifying information from the coding tag to the recording tag, and/or solid support(s).

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A-1D is a schematic depicting an exemplary workflow for providing polypeptides in a tissue section with recording tags and steps for spatial analysis utilizing one or more molecular probes associated with a detectable label and a probe tag.

FIG. 2A-2F is a schematic depicting an exemplary workflow for providing polypeptides in a tissue section with recording tags and steps for spatial analysis utilizing spatial probes (e.g., beads) associated with a spatial tag and one or more molecular probes associated with a probe tag.

### DETAILED DESCRIPTION

Provided herein are methods and kits for analyzing a macromolecule or a plurality of macromolecules, e.g., peptides, polypeptides, and proteins. In some embodiments, the analysis employs barcoding and nucleic acid encoding of molecular recognition events and/or detectable labels. In some aspects, the macromolecule is a polypeptide. In some embodiments, the method provides information (e.g., identity, characteristics, location in the spatial sample, spatial distribution, density, location) regarding the macromolecule. In some cases, the identity and/or at least a partial sequence of the polypeptide or the protein in the spatial sample is obtained from performing the method and may be associated with spatial information regarding the spatial tag in the spatial sample (such as its location in the sample).

Current methods for identifying and analyzing molecules from a sample while providing information regarding characteristics of the sample, for example, the presence, absence, concentration, and/or spatial distribution of multiple biological targets of interest in a sample are limited. For example, known approaches for identifying proteins while retaining other sample or spatial information is not appropriate for analyzing a large number of unknown proteins within a sample. Some current techniques may detect only a few targets at one time, require use of multiple samples, and/or require further processes for analysis, including analysis of the identity and/or sequence of the proteins. Accordingly, there remains a need in the art for improved techniques relating to multiplex macromolecule (e.g., polypeptide) analysis and/or characterization that is highly-parallelized, accurate, sensitive, and/or high-throughput with an option to also further perform analysis and/or sequencing of proteins.

In some embodiments, the present disclosure provides, in part, methods for analyzing macromolecules, (e.g., peptides, polypeptides, and proteins) including obtaining spatial information (e.g., distribution and/or location) related to the macromolecule to use with methods of highly-parallel, high throughput digital macromolecule characterization and quantitation, with direct applications to protein and peptide characterization and sequencing. In some embodiments, the method provides spatial information (e.g., position or location) of one or more polypeptides in a spatial sample and the identity or a partial sequence of the polypeptide(s) analyzed.

Provided herein are methods of analyzing a macromolecule (e.g., protein, polypeptide, or peptide) comprising steps: (a) providing a spatial sample comprising a macromolecule associated with a recording tag; (b1) providing a spatial probe comprising a spatial tag to the spatial sample; (b2) assessing the spatial tag in situ to obtain the spatial location of the spatial tag in the spatial sample; (b3) extending the recording tag by transferring information from the spatial tag in the spatial probe to the recording tag; (c1) binding a molecular probe comprising a probe tag to the macromolecule or a moiety in proximity to the macromolecule in the spatial sample; (c2) extending the recording tag by transferring information from the probe tag in the molecular probe to the recording tag, wherein transferring information from the spatial tag and/or probe tag to the recording tag generates an extended recording tag; (d) determining at least the sequence of the probe tag and spatial tag in the extended recording tag; and (e) correlating the sequence of the spatial tag determined in step (d) with the spatial tag assessed in step (b2); thereby associating information from the sequence of the extended recording tag or a portion thereof, e.g., the information from the spatial tag and/or probe tag, determined in step (d) with the spatial location of the spatial probe assessed in step (b2). In some embodiments, step (a) comprises providing the spatial sample with a plurality of recording tags. In some of any such embodiments, the macromolecules are polypeptides. In some embodiments, the method further includes performing a polypeptide analysis assay.

Provided herein are methods and kits for analyzing a macromolecule, (e.g., peptide, polypeptide, and protein) including steps: providing a spatial sample comprising a macromolecule with a recording tag; (b) binding a molecular probe comprising a detectable label and a probe tag to the macromolecule or a moiety in proximity to the macromolecule in the spatial sample; (c) transferring information from the probe tag in the molecular probe to the recording tag to generate an extended recording tag; (d) assessing, e.g., observing, the detectable label to obtain spatial information of the molecular probe; (e) determining at least the sequence of the probe tag in the extended recording tag; and (f) correlating the sequence of the probe tag determined in step (e) with the molecular probe; thereby associating information from the sequence determined in step (e) with its spatial information determined in step (d). In some embodiments, the method includes performing a polypeptide analysis assay. In some embodiments, a macromolecule analysis assay is not performed prior to step (e) and (f). In other embodiments, a macromolecule analysis assay is performed prior to steps (e) and (O.

Provided herein are methods and kits for analyzing a macromolecule including steps: (a) providing a spatial sample comprising a macromolecule with a recording tag; (b) binding a molecular probe comprising a detectable label and a probe tag to the macromolecule or a moiety in proximity to the macromolecule in the spatial sample; (c) transferring information from the probe tag in the molecular probe to the recording tag to generate an extended recording tag; (d) assessing, e.g., observing, the detectable label to obtain spatial information of the molecular probe; (e) determining at least the sequence of the probe tag in the extended recording tag; and (f) correlating the sequence of the probe tag determined in step (e) with the molecular probe; thereby associating information from the sequence determined in step (e) with its spatial information determined in step (d).

Also provided are kits for use with any of the provided methods. In some embodiments, the kits comprise one or more of the following components: spatial probe(s), spatial tag(s), molecular probe(s), probe tag(s), reagent(s) for sequencing, reagent(s) for performing nucleic acid extension recoding tag(s), reagent(s) for attaching or transferring the recording tag, binding agent(s), reagent(s) for transferring identifying information from the probe tag or spatial tag to the recording tag, reagent(s) for transferring identifying information from the coding tag to the recording tag, and/or solid support(s).

In some of any such embodiments, the macromolecules are polypeptides. In some embodiments, a plurality of molecular probes are used in the method to bind the spatial sample and a plurality of spatial probes are provided to associate with the spatial sample. In some embodiments, the molecular probes bind to nucleic acids, polypeptides, or other macromolecules in the spatial sample. In some embodiments, more than one cycle of binding with molecular probes and transferring information from the molecular probe to the recording tag is performed. The transferring of information to the recording tag from one or more probe tags forms an extended recording tag by using any suitable transfer methods.

The method may also include providing a plurality of spatial probes to the spatial sample. In some embodiments, the spatial probe comprises a plurality of spatial tags, and the spatial tags comprise a barcode. In some embodiments, the spatial probes (with associated barcodes) are randomly distributed among the spatial sample. In some cases, the method includes determining, analyzing, and/or sequencing the spatial tag in situ to obtain the spatial location of the spatial tag in the spatial sample. In some embodiments, the methods includes a step of decoding the barcodes associated with the spatial probes in situ. In some embodiments, the method allows association of spatial information gained from assessing the spatial tag in situ to obtain the spatial location of the spatial tag in the spatial sample with any information recorded on the extended recording tag.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide or protein after its translation by ribosomes is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, carbohydrate, or a small molecule that binds to, associates, unites with, recognizes, or combines with an analyte, e.g., a macromolecule or a component or feature of a macromolecule. A binding agent may form a covalent association or non-covalent association with the analyte, e.g., a macromolecule or component or feature of a macromolecule. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a macromolecule (e.g., a single amino acid of a peptide) or bind to a plurality of linked subunits of a macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may for example bind to a chemically modified or labeled amino acid over a non-modified or unlabeled amino acid. For example, a binding agent may for example bind to an amino acid that has been modified with an acetyl moiety, guanyl moiety, dansyl moiety, PTC moiety, DNP moiety, SNP moiety, etc., over an amino acid that does not possess said moiety. A binding agent may bind to a post-translational modification of a polypeptide molecule. A binding agent may exhibit selective binding to a component or feature of an analyte, such as a macromolecule (e.g., a binding agent may selectively bind to one of the 20 possible natural amino acid residues and bind with very low affinity or not at all to the other 19 natural amino acid residues). A binding agent may exhibit less selective binding, where the binding agent is capable of binding a plurality of components or features of an analyte, such as a macromolecule (e.g., a binding agent may bind with similar affinity to two or more different amino acid residues). A binding agent comprises a coding tag, which may be joined to the binding agent by a linker.

As used herein, the term “fluorophore” refers to a molecule which absorbs electromagnetic energy at one wavelength and re-emits energy at another wavelength. A fluorophore may be a molecule or part of a molecule including fluorescent dyes and proteins. Additionally, a fluorophore may be chemically, genetically, or otherwise connected or fused to another molecule to produce a molecule that has been “tagged” with the fluorophore.

As used herein, the term “linker” refers to one or more of a nucleotide, a nucleotide analog, an amino acid, a peptide, a polypeptide, or a non-nucleotide chemical moiety that is used to join two molecules. A linker may be used to join a binding agent with a coding tag, a recording tag with a polypeptide, a polypeptide with a solid support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

The terminal amino acid at one end of the peptide chain that has a free amino group is referred to herein as the “N-terminal amino acid” (NTAA). The terminal amino acid at the other end of the chain that has a free carboxyl group is referred to herein as the “C-terminal amino acid” (CTAA). An N-terminal diamino acid may comprise the N-terminal amino acid and the penultimate N-terminal amino acid. A C-terminal diamino acid is similarly defined for the C-terminus. The amino acids making up a peptide may be numbered in order, with the peptide being “n” amino acids in length. As used herein, NTAA is considered the nt^{h} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be functionalized with a chemical moiety.

As used herein, the term “nucleic acid barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for or regarding a macromolecule, a polypeptide, a binding agent, a set of binding agents from a binding cycle, a sample of polypeptides, a set of samples, macromolecules (e.g., polypeptides) within a compartment (e.g., droplet, bead, or separated location), macromolecules (e.g. polypeptides) within a set of compartments, a fraction of macromolecules (e.g. polypeptides), a set of polypeptide fractions, a spatial region or set of spatial regions, a library of macromolecules or polypeptides, a molecular probe or a set of molecular probes, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

As used herein “peptide barcode” or “amino acid barcode” refers to a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode can be distinguished from other peptide barcodes by having a different length, sequence, or other physical property (for example, hydrophobicity). A peptide barcode can provide a unique identifier tag or origin information for or regarding a macromolecule, a polypeptide, a binding agent, a set of binding agents from a binding cycle, a sample of polypeptides, a set of samples, a location (e.g., a spatial location), macromolecules (e.g., polypeptides) within a compartment (e.g., droplet, bead, or separated location), macromolecules (e.g. polypeptides) within a set of compartments, a fraction of molecules, a set of fractions, a spatial region or set of spatial regions, a library of macromolecules or polypeptides, a molecular probe or a set of molecular probes, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated.

A “sample barcode”, also referred to as “sample tag” identifies from which sample a polypeptide derives.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “encoder sequence” or “encoder barcode” refers to a nucleic acid molecule of about 2 bases to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder sequence may uniquely identify its associated binding agent. In certain embodiments, an encoder sequence is provides identifying information for its associated binding agent and for the binding cycle in which the binding agent is used. In other embodiments, an encoder sequence is combined with a separate binding cycle-specific barcode within a coding tag. Alternatively, the encoder sequence may identify its associated binding agent as belonging to a member of a set of two or more different binding agents. In some embodiments, this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In another example, a common encoder sequence is used for polyclonal antibodies, which comprises a mixture of antibodies that recognize more than one epitope of a protein target, and have varying specificities. In other embodiments, where an encoder sequence identifies a set of possible binding agents, a sequential decoding approach can be used to produce unique identification of each binding agent. This is accomplished by varying encoder sequences for a given binding agent in repeated cycles of binding (see, Gunderson et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder sequence) provides the uniquely identifying information for the binding agent. Preferably, the encoder sequences within a library of binding agents possess the same or a similar number of bases.

As used herein the term “binding cycle specific tag”, “binding cycle specific barcode”, or “binding cycle specific sequence” refers to a unique sequence used to identify a library of binding agents used within a particular binding cycle. A binding cycle specific tag may comprise about 2 bases to about 8 bases (e.g., 2, 3, 4, 5, 6, 7, or 8 bases) in length. A binding cycle specific tag may be incorporated within a binding agent's coding tag as part of a spacer sequence, part of an encoder sequence, part of a UMI, or as a separate component within the coding tag.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the macromolecule (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a polypeptide, information from a coding tag linked to a binding agent can be transferred to the recording tag associated with the polypeptide while the binding agent is bound to the polypeptide. In other embodiments, after a binding agent binds to a polypeptide, information from a recording tag associated with the polypeptide can be transferred to the coding tag linked to the binding agent while the binding agent is bound to the polypeptide. A recoding tag may be directly linked to a macromolecule, e.g., a polypeptide, linked to a macromolecule, e.g., a polypeptide, via a multifunctional linker, or associated with a macromolecule, e.g., a polypeptide, by virtue of its proximity (or co-localization) on a solid support. A recording tag may be linked via its 5′ end or 3′ end or at an internal site, if the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length providing a unique identifier tag for each polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5” or “sense”. The term “reverse” when used in context with a “universal priming site” or “universal primer” may also be referred to as “3” or “antisense”.

As used herein, the term “extended recording tag” refers to a recording tag to which information of at least one binding agent's coding tag (or its complementary sequence) has been transferred following binding of the binding agent to a macromolecule, e.g., a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “extended coding tag” refers to a coding tag to which information of at least one recording tag (or its complementary sequence) has been transferred following binding of a binding agent, to which the coding tag is joined, to a polypeptide, to which the recording tag is associated. Information of a recording tag may be transferred to the coding tag directly (e.g., ligation), or indirectly (e.g., primer extension). Information of a recording tag may be transferred enzymatically or chemically. In certain embodiments, an extended coding tag comprises information of one recording tag, reflecting one binding event. As used herein, the term “di-tag” or “di-tag construct” or “di-tag molecule” refers to a nucleic acid molecule to which information of at least one recording tag (or its complementary sequence) and at least one coding tag (or its complementary sequence) has been transferred following binding of a binding agent, to which the coding tag is joined, to a polypeptide, to which the recording tag is associated. Information of a recording tag and coding tag may be transferred to the di-tag indirectly (e.g., primer extension). Information of a recording tag may be transferred enzymatically or chemically. In certain embodiments, a di-tag comprises a UMI of a recording tag, a compartment tag of a recording tag, a universal priming site of a recording tag, a UMI of a coding tag, an encoder sequence of a coding tag, a binding cycle specific barcode, a universal priming site of a coding tag, or any combination thereof.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a paramagnetic bead, a glass bead, or a controlled pore bead. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” a macromolecule, means to identify, quantify, characterize, distinguish, or a combination thereof, all or a portion of the components of the macromolecule. For example, analyzing a peptide, polypeptide, or protein includes determining all or a portion of the amino acid sequence (contiguous or non-continuous) of the peptide. Analyzing a macromolecule also includes partial identification of a component of the macromolecule. For example, partial identification of amino acids in the macromolecule protein sequence can identify an amino acid in the protein as belonging to a subset of possible amino acids. Analysis typically begins with analysis of the nt^{h} NTAA, and then proceeds to the next amino acid of the peptide (i.e., n−1, n−2, n−3, and so forth). This is accomplished by cleavage of the n^{th} NTAA, thereby converting the (n−1)^{th} amino acid of the peptide to an N-terminal amino acid (referred to herein as the “(n−1)^{th} NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. METHODS OF ANALYZING MACROMOLECULES

Provided herein is a method of analyzing a macromolecule comprising providing a spatial sample comprising a macromolecule associated with a recording tag at spatial location; assessing the spatial location of the macromolecule in the spatial sample in situ; binding a molecular probe comprising and a probe tag to the macromolecule or a moiety in proximity to the macromolecule in the spatial sample; extending the recording tag by transferring information from the probe tag in the molecular probe to the recording tag, wherein transferring information from the probe tag to the recording tag generates an extended recording tag; determining at least the sequence of the probe tag in the extended recording tag; and correlating the sequence of the probe tag determined with the molecular probe and/or spatial location assessed in situ. In some cases, the method includes correlating the sequence of the probe tag determined with the molecular probe (e.g. the identity or binding information/characteristics regarding the molecular probe that bound). Using the method, the information from the sequence of the extended recording tag or a portion thereof determined can be associated with the spatial location assessed in situ. In some aspects, assessing the spatial location of the macromolecule in the spatial sample in situ is performed using imaging based approaches, e.g. fluorescent imaging, combinatorial hybridization-based approaches and/or in situ NGS sequencing.

In some embodiments, the recording tag may comprise spatial information. For example, the recording tag may comprise a spatial tag. The recording tag providing spatial information may be in the form of a UMI. In some aspects, the method includes a first step of providing a spatial sample comprising a macromolecule associated with a recording tag, wherein the recording tag comprises spatial information, such as a spatial tag. The spatial tag may be directly or indirectly associated or joined to the recording tag. The method may also include analyzing or assessing spatial tag in situ. The analyzing or assessing of the spatial tag may be performed using a microscope-based method. In some cases, the analyzing or assessing of the spatial tag includes sequencing, e.g., sequencing by ligation, single molecule sequencing, single molecule fluorescent sequencing, or sequencing by probe detection.

In general, the methods provided include assessing spatial information, either by decoding a spatial tag in situ or by assessing, e.g., observing, a detectable label to obtain spatial information of the location of the macromolecule or a moiety in proximity to the macromolecule. The spatial information may be in the form of providing spatial tags to the sample, wherein the spatial tags are transferred to the macromolecule, such as to the recording tag associated with the macromolecule. In some aspects, the decoding of the spatial tag can be performed before or after transferring the spatial tag to the recording tag. In some specific cases, decoding of the spatial tag includes assessing the spatial location of the macromolecule in the spatial sample in situ.

In some embodiments, assessing the spatial location of the macromolecule in the spatial sample is performed by providing a spatial probe comprising a spatial tag to the spatial sample and assessing the spatial tag in situ to obtain the spatial location of the spatial tag in the spatial sample. By observing the detectable label on the molecular probe or by assessing the spatial tag of the spatial probes in situ, both methods allow a way to observe (e.g., by imaging) the spatial location of the macromolecules in the sample, as described in section II.

In some embodiments, assessing the spatial location of the macromolecule in the spatial sample in situ is performed by binding a molecular probe comprising a detectable label and a probe tag to the macromolecule or a moiety in proximity to the macromolecule in the spatial sample and assessing, e.g., observing, the detectable label to obtain spatial information of the molecular probe, as described in section III.

In some embodiments, the present disclosure provides a recording method for capturing multiple sources of information into a recording tag, including spatial information and information from one or more molecular probes. The methods of the present invention also permit the detection, analysis, and/or sequencing of a plurality of peptides (two or more peptides) simultaneously, e.g., multiplexing. Simultaneously as used herein refers to detection, quantitation or sequencing of a plurality of peptides in the same assay. The plurality of peptides assessed can be present in the same sample, e.g., biological sample, or different samples. The plurality of peptides assessed can be different peptides, or the same peptides in different samples. The plurality is 10 or more peptides, 50 or more peptides, 100 or more peptides, 500 or more peptides, 1000 or more peptides, 10,000 or more peptides, 100,000 or more peptides or 1,000,000 or more peptides. In some aspects, the provided methods allow release and processing of the sample (or portions thereof) after assessing or determining spatial information, and further allow other steps to be performed on the sample after release.

### II. ANALYZING MACROMOLECULES USING SPATIAL PROBES

Provided herein are methods of analyzing a macromolecule (e.g., protein, polypeptide, or peptide) comprising steps: (a) providing a spatial sample comprising a macromolecule associated with a recording tag; (b1) providing a spatial probe comprising a spatial tag to the spatial sample; (b2) assessing the spatial tag in situ to obtain the spatial location of the spatial tag in the spatial sample; (b3) extending the recording tag by transferring information from the spatial tag in the spatial probe to the recording tag; (c1) binding a molecular probe comprising a probe tag to the macromolecule or a moiety in proximity to the macromolecule in the spatial sample; (c2) extending the recording tag by transferring information from the probe tag in the molecular probe to the recording tag, wherein transferring information from the spatial tag and/or probe tag to the recording tag generates an extended recording tag; (d) determining at least the sequence of the probe tag and spatial tag in the extended recording tag; and (e) correlating the sequence of the spatial tag or a portion thereof, e.g., the information from the spatial tag and/or probe tag, determined in step (d) with the spatial tag assessed in step (b2); thereby associating information from the sequence of the extended recording tag determined in step (d) with the spatial location of the spatial probe assessed in step (b2). In some embodiments, the macromolecule is a polypeptide. In some aspects, a plurality of macromolecules in a spatial sample is provided with recording tags in step (a). In some embodiments, the recording tags may be associated or attached, directly or indirectly to the macromolecules or other moieties in the spatial sample. In some other embodiments, the recording tags are not associated or attached, directly or indirectly to the macromolecules or other moieties in the spatial sample but are held in place in a matrix, scaffold, or substance applied to the spatial sample.

In some embodiments, the method includes determining at least the sequence of the probe tag and the spatial tag in the extended recording tag. In some aspects, the sequence of a series of probe tags (e.g., barcodes) and a series of probe tags (e.g., barcodes) is used to associate information contained in the extended recording tag with the spatial location of the associated macromolecule. In some embodiments, the information of the molecular probe(s), including target of the molecular probe(s) and other characteristics of the macromolecule bound by the molecular probe(s) can be associated with the spatial location of spatial tag assessed in situ. In some embodiments, the sample is sequentially bound by two or more molecular probes, removing any previous probe prior to binding of any subsequent probes.

Some of the steps of the provided methods may be reversed or performed in various orders. For example, step (b2) can be performed either before or after step (b3). In some embodiments, one or more of the steps may be repeated. In a preferred embodiment, the binding of the molecular probe and extending the recording tag by transferring information from the probe tag associated with the molecular probe to the recording tag is performed prior to providing a spatial probe comprising a spatial tag to the spatial sample. For example, steps (c1) and (c2) can be repeated two or more times in sequential order prior to performing steps (d) and (e). In one example, steps (a), (c1), (c2), (b1), (b2), (b3), (d), and (e) occur in sequential order. The method may include removing any molecular probe prior to providing a spatial probe to the spatial sample; or removing any spatial probe from the sample prior to binding the sample with a molecular probe. In some embodiments, the method includes removing the molecular probe from the spatial sample prior to repeating step (c1). In some embodiments, step (a) is performed prior to steps (1)1), (b2), (b3), (c1), (c2), (d), and (e). In some cases, step (b1) is performed prior to steps (b2), (d), and (e). In some examples, steps (c1) and (c2) are performed prior to steps (d) and step (e). In some aspects, steps (c1) and (c2) are performed prior to or after steps (b1), (b2), and/or (b3). In some cases, step (d) is performed prior to step (e). In some embodiments, step (b2) is performed after steps (a), (b1), (b3), (c1), and/or (c2). In some embodiments, step (e) is performed after steps (a) (b1), (b2), (b3), (c1), (c2), and (d). In some embodiments, the macromolecule analysis assay is not performed. In some embodiments, the method includes performing a macromolecule analysis assay after steps (1)1), (b2), (b3), (e1), and (c2). In some embodiments, the macromolecule analysis assay is performed before steps (d) and (e).

In some embodiments, the extended recording tag analyzed comprises information from a plurality of probe tags sequentially transferred to the recording tag. In some embodiments, the extended recording tag comprises information from at least one probe tag and spatial tag. In some further embodiments, the extended recording tags comprise information transferred from at least one probe tag, at least one spatial tag, and at least one coding tag.

In some of the embodiments provided, the binding of a molecular probe to the spatial sample and transferring information from the probe tag to the recording tag can be repeated one or more times. In some aspects, any previous molecular probes may be removed after transferring information from the probe tag to the recording tag and prior to binding of the sample with any subsequent molecular probes. In some embodiments of the provided methods, the molecular probe binds to the spatial sample by binding to a macromolecule in the spatial sample or binding to a moiety in proximity to the macromolecule in the spatial sample. In some embodiments, the molecular probe binds to a moiety that is bound to, associated with or complexed with the macromolecule in the spatial sample. In some embodiments, a plurality of molecular probes is applied to the spatial sample. In some embodiments, the molecular probe is capable of selective and/or specific binding. In some embodiments, the molecular probe binds to a macromolecule in complex with other macromolecules. For example, the molecular probe may bind to a nucleic acid in a complex with a polypeptide and the polypeptide is associated with a recording tag. In some specific embodiments, the molecular probe binds to the polypeptide to which the recording tag is associated.

In some aspects, the molecular probe comprises a probe tag which may comprise any sequenceable molecule. In some examples, the probe tag comprises a barcode. The information of the probe tag is transferred in any suitable manner to the recording tag. In some embodiments, the information from one probe tag may be transferred to two or more recording tags. In some embodiments, the information from two or more probe tags may be transferred to one recording tag.

In some embodiments, a plurality of spatial probes is applied to the spatial sample. In some aspects, the spatial probe comprises a spatial tag attached via a cleavable linker to a support (e.g. a bead). In some embodiments, the spatial probe does not exhibit selective and/or specific binding. For example, a plurality of spatial probes are randomly distributed onto a spatial sample for transferring the spatial tags to the recording tags. In some embodiments, the spatial probe associates with the sample non-specifically via adhesive forces such as charge interaction, DNA hybridization, or reversible chemical coupling. In some embodiments, the spatial probes distributed or applied to the spatial sample are closely packed in a confined space or area. In some examples, the spatial probes are provided as an array of immobilized beads. For example, the spatial tag associates with a recording tag via hybridization of a sequence complementary to the recording tag comprised in the spatial tag (or a portion thereof). The spatial probe comprises a spatial tag which may comprise any sequenceable molecule. In some examples, the spatial tag comprises a barcode. The information of the spatial tag is transferred in any suitable manner to the recording tag.

In some embodiments, a spatial sample includes a biological sample. For example, the spatial sample may include macromolecules, cells, and/or tissues obtained from a subject. In some examples, the spatial sample is derived from a sample such as an intact tissue or a liquid sample. For example, the liquid sample may be spread deposited onto a surface prior to performing the methods. In some examples, the spatial sample is processed prior to binding of the molecular probes or spatial probes to the spatial sample, such as by treating the sample with a permeabilizing, fixing, and/or cross-linking reagent.

In some embodiments, after generating an extended recording tag comprising information from probe tags and spatial tags, a sample containing a plurality of macromolecules may be treated to allow release of the macromolecules. Optionally, the spatial sample or any portion thereof can be removed from a solid support after transfer of information from at least one probe tag and spatial tag to the recording tag. Thus, a method of the present disclosure can include a step of washing a solid support to remove macromolecules, cells, tissue or other materials from the spatial sample. Removal of the spatial sample or any portion thereof can be performed using any suitable technique and will be dependent on the sample. In some cases, the solid support can be washed with water containing various additives, such as surfactants, detergents, enzymes (e.g., proteases and collagenases), cleavage reagents, or the like, to facilitate removal of the specimen. In some embodiments, the solid support is treated with a solution comprising a proteinase enzyme. In some embodiments, macromolecules are released during or after the specimen is removed from the solid support. The release of the sample from a solid support may be performed by physical or chemical treatment, including but not limited to trypsin digest, scraping, chemical dissociation, etc. In some embodiments, after generating an extended recording tag comprising information from probe tags and spatial tags (and optionally from coding tags), the extended recording tags are released from the spatial sample. In some embodiments, after generating an extended recording tag comprising information from probe tags and spatial tags (and optionally from coding tags), the extended recording tags are amplified. In some embodiments, released macromolecules attached to the extended recording tags may be used in a macromolecule analysis assay.

In some embodiments, the method further include performing a macromolecule analysis assay. In some embodiments, the macromolecule (e.g., polypeptide or polynucleotide) analysis assay is performed in situ. In some other embodiments, the macromolecule analysis assay is performed after the macromolecules with the associated recording tags are released from the spatial sample. In some examples, the macromolecule analysis assay comprises a polypeptide analysis assay. In some of any such embodiments, the macromolecule analysis assay includes one or more cycles of contacting the macromolecule with a binding agent capable of binding to the macromolecule, wherein the binding agent comprises a coding tag with identifying information regarding the binding agent; and transferring the information of the coding tag to the recording tag to generate an extended recording tag. The identifying information from the binding agent is transferred to the recording tag associated with the polypeptide which also comprises information transferred from the probe tag and spatial tag. Thus, in some embodiments, the extended recording tag comprises information from one or more probe tags, one or more spatial tags, one or more coding tags, and optionally any other nucleic acid components.

In some embodiments, the macromolecule analysis assay comprises determining the sequence of at least a portion of a macromolecule (e.g., polypeptide or polynucleotide). In some cases, the analysis method may include performing any of the methods as described in International Patent Publication No. WO 2017/192633. In some cases, the sequence of a polypeptide is analyzed by construction of an extended nucleic acid sequence which represents the polypeptide sequence or a portion thereof, such as an extended nucleic acid onto the recording tag (or any additional barcodes or tags attached thereto). In some embodiments, the method further comprising determining at least a portion of the sequence of the macromolecule or the identity of the macromolecule and associating with the spatial location assessed in step (b2).

An exemplary workflow for analyzing polypeptides may include the following: a spatial sample of a tissue section is provided on a solid support. The macromolecules (e.g., proteins) of the spatial sample are labeled with recording tags. The recording tags may include a universal priming site that is useful for later amplification. A plurality of molecular probes each comprising a probe tag is applied to the spatial sample and binds to the sample. The information from the probe tags are transferred to recording tags attached to the proteins by a suitable method, such as by ligation or extension. After transfer of the information from the probe tags, the molecular probes may be removed, released, or washed. Optionally, additional rounds of binding with molecular probes and transferring information from the probe tags to the recording tags may be performed. A plurality of spatial probes each comprising a bead with a plurality of probe tags (containing barcodes) attached via a photo-cleavable linker to the bead is randomly distributed onto the spatial sample. The spatial tags on the spatial probe (e.g., bead) are determined in situ to provide information of the spatial location of the spatial tag in the sample. The barcodes are cleaved from the other components of the spatial probe (e.g., bead) and allowed to diffuse into the tissue section and hybridize with complementary DNA on recording tags attached to proteins. The tissue section is exposed to a polymerase extension mix to transfer barcode information from the hybridized barcode serving as a template to the DNA recording tag. After transfer of information from the probe tags and spatial tags onto the extended recording tag, the polypeptides and attached recording tags are released from the spatial sample. In an optional step, the polypeptides are digested and a polypeptide analysis assay may be optionally performed, the polypeptides and associated recording tags (comprising information from the spatial and probe tags) can be immobilized randomly on a single molecule sequencing substrate (e.g., beads) at an appropriate intramolecular spacing. If a polypeptide analysis assay is performed on the polypeptides associated with the extended recording tag, further identifying information from coding tags is transferred to the extended recording tags. At least a portion of the sequence of the extended recording tag (with the information from the spatial and probe tag comprised therein) is determined. Using this workflow, information on the polypeptide associated with the extended recording tag is associated with spatial location of the polypeptide in the spatial sample from which it originated.

A method set forth herein can include one or more steps of acquiring an image of a spatial sample (e.g., a biological specimen). In some embodiments, two or more images of the spatial sample or a portion thereof are obtained. In some cases, the method includes comparing, aligning, and/or overlaying two or more images. The imaging may be performed on a spatial sample that is in contact with a solid support. An image can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g., different regions of a tissue, different cells, specific subcellular components or the like). In other embodiments, a biological specimen can be imaged without staining. In some embodiments, the method includes overlaying two or more images obtained of the spatial sample to produce an composite image.

A detection system including microscopes configured for light, bright field, dark field, phase contrast, fluorescence, reflection, interference, and/or confocal imaging may be used in conjunction with one or more steps of the method. The detection system may include an electron spin resonance (ESR) detection system, a charge coupled device (CCD) detection system (e.g., for radioisotopes), a fluorescent detection system, an electrical detection system, a photographic film detection system, a chemiluminescent detection system, an enzyme detection system, an atomic force microscopy (AFM) detection system (for detection of microbeads), a scanning tunneling microscopy (STM) detection system (for detection of microbeads), an optical detection system, a near field detection system, or a total internal reflection (TIR) detection system.

In some embodiments, the method includes correlating locations in an image of the sample with spatial tags. Other characteristics of the spatial sample containing a biological specimen that are identifiable in the image can be obtained. Any of a variety of morphological characteristics can be obtained, including for example, cell shape, cell size, tissue shape, staining patterns, presence of particular proteins (e.g. as detected by immunohistochemical stains) or other characteristics that are routinely evaluated in pathology or research applications. Accordingly, the biological state of a tissue or its components as determined by visual observation can also be obtained.

A. Samples

In one aspect, the present disclosure relates to the analysis of macromolecules from a sample. A macromolecule can be a large molecule composed of smaller subunits. In certain embodiments, a macromolecule is a protein, a protein complex, polypeptide, peptide, nucleic acid molecule, carbohydrate, lipid, macrocycle, or a chimeric macromolecule. In some embodiments, the macromolecule is a protein, a polypeptide, or a peptide.

In some embodiments, the macromolecules (e.g., proteins, polypeptides, or peptides) are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells.

The sample may be a spatial sample, from which information regarding the spatial arrangement and/or location of anatomical features, morphological features, cellular features, and/or subcellular features may be desired. In some embodiments, the sample is further processed by methods known in the art. For example, a sample is processed to remove, clear, or isolate cellular material (e.g., by centrifugation, filtration, etc.). The spatial sample may refer to a biological sample arranged such that constituents, portions, or regions of the sample may be referenced spatially (e.g. arranged in a planar format such as a tissue section on a slide).

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids. In some of any of the provided embodiments, a sample, such as a fluid sample, may be deposited on a surface. For example, a liquid sample may be processed to prepare a cell spread on a solid surface such as a slide. In some embodiments, a sample or a portion thereof (such as analytes or cells obtained from the sample) may be deposited in a polymer resin. In some cases, the polymer resin comprises a hydrogel-forming natural or synthetic polymer.

In some embodiments, the sample is a tissue sample. A tissue can be prepared in any convenient or desired way for its use in any of the methods described herein. Fresh, frozen, fixed or unfixed tissues can be used. A tissue can be prepared, fixed or embedded using methods described herein or known in the art (Fischer et al., CSH Protoc (2008) pdb prot4991; Fischer et al., CSH Protoc (2008) pdb top36; Fischer et al., CSH Protoc. (2008) pdb.prot4988). The tissue can be freshly excised from an organism or it may have been previously preserved for example by freezing, embedding in a material such as paraffin (e.g. formalin fixed paraffin embedded samples), formalin fixation, infiltration, dehydration or the like. In some examples, a matrix-forming material can be used to encapsulate a biological sample, such as a tissue sample. In some cases, the sample is embedded in a paraffin block. For example, the spatial sample may be a formalin-fixed, paraffin-embedded (FFPE) section. Optionally, a tissue section can be attached to a solid support, for example, using techniques and compositions exemplified herein with regard to attaching nucleic acids, cells, viruses, beads or the like to a solid support (Ramos-Vera et al., J Vet Diagn Invest. (2008) 20(4):393-413). As a further option, a tissue can be permeabilized and the cells of the tissue lysed when the tissue is in contact with a solid support. Standard conditions and reagents may be used for tissue permeabilization including incubation with any suitable detergents, Triton X-100, ethoxylated nonylphenol (Tergitol-type NP-40), Tween 20, Saponin, Digitonin, or acetone (Fischer et al., CSH Protoc (2008) pdb top36).

In some embodiments, the sample is a “planar sample” that is substantially planar, i.e., two dimensional. In some embodiments, a sample is deposited in a substrate or deposited on a solid surface. In some embodiments, the sample is a three dimensional sample. In some examples, a material or substrate (e.g. glass, metal, ceramics, organic polymer surface or gel) may contain cells or any combination of biomolecules derived from cells, such as proteins, nucleic acids, lipids, oligo/polysaccharides, biomolecule complexes, cellular organelles, extracellular vesicles, cellular debris or excretions. In some embodiments, the planar cellular sample can be made by, e.g., depositing cells or portions thereof on a planar surface, e.g., by centrifugation, by cutting a three dimensional object that contains cells into sections and mounting the sections onto a planar surface, i.e., producing a tissue section. In some embodiments, the sample is a tissue section that refers to a piece of tissue that has been obtained from a subject, fixed, sectioned (e.g., cryosectioning), and mounted on a planar surface, e.g., a microscope slide.

In some embodiments, the spatial sample (e.g., specimen or tissue sample) is treated to expand the sample. In some aspects, the spatial sample is preserved and expanded isotropically using a chemical process. For example, a tissue sample may be treated to attach anchors to biomolecules in the spatial sample, perform in situ polymer synthesis, perform mechanical homogenization, and perform specimen expansion (See e.g., Zhao et al., Nature Biotechnology (2017) 35(8):757-764; Chang et al., Nature Methods (2017) 14:593-599; Chang et al., Nature Methods (2016) 13(8):679-84; Tillberg et al., Nature Biotechnology (2016) 34:987-992; Chen et al., Science (2015) 347(6221):543-548; Asano et al., Current Protocols in Cell Biology (2018) 80(1):e56; Wassie et al., Nature Methods (2018) 16(1):33-41; Boyden et al., Mater. Horiz., (2019) 6, 11-13; Alon et al., FEB S J. 2019 April; 286(8):1482-1494. Karagiannis et al., Current Opinion in Neurobiology (2018) 50:56-63; Gao et al., BMC Biology (2017) 15(1):50).

In some embodiments, the method includes obtaining and preparing macromolecules (e.g., polypeptides and proteins) from a single cell type or multiple cell types. In some embodiments, the sample comprises a population of cells. In some embodiments, the macromolecules (e.g., proteins, polypeptides, or peptides) are from a cellular or subcellular component, an extracellular vesicle, an organelle, or an organized subcomponent thereof. In some embodiments, the polypeptides are from one or more packaging of molecules (e.g., separate components of a single cell or separate components isolated from a population of cells, such as organelles or vesicles). The macromolecules (e.g., proteins, polypeptides, or peptides) may be from organelles, for example, mitochondria, nuclei, or cellular vesicles. In one embodiment, one or more specific types of single cells or subtypes thereof may be isolated. In some embodiments, the spatial samples may include but are not limited to cellular organelles, (e.g., nucleus, golgi apparatus, ribosomes, mitochondria, endoplasmic reticulum, chloroplast, cell membrane, vesicles, etc.).

1. Fixation and Permeabilization

In some embodiments, the methods provided herein further include one or more fixing (e.g., cross linking) and/or permeabilizing steps. In certain embodiments, the sample comprising macromolecules (e.g., proteins, polypeptides, or peptides) for analysis may be fixed and/or permeabilized. For example, holes or openings may be formed in membranes of the cells and/or any subcellular components. The cells, subcellular structures and components, or biomolecules may be fixed using any number of reagents including but not limited to formalin, methanol, ethanol, paraformaldehyde, formaldehyde, methanol: acetic acid, glutaraldehyde, bifunctional crosslinkers such as bis(succinimidyl)suberate, bis(succinimidyl)polyethyleneglycole etc.

In some examples, the methods of treating proteins and analyzing proteins provided herein may comprise fixing the sample at any step in the method. In some cases, fixing the sample is performed prior to permeabilizing the sample (e.g., permeabilizing the cells or other membranes). In some examples, fixing the sample is performed after permeabilizing the sample. In some embodiments, the sample is fixed or cross linked prior to providing a protein in a spatial sample with a recording tag. In some embodiments, the sample is permeabilized prior to binding the spatial sample with one or more molecular probes.

In some embodiments, the samples may be fixed or cross-linked such that the cellular and subcellular components are immobilized or held in place. In some embodiments, the macromolecules in the sample (e.g., DNA, RNA, proteins, polypeptides, lipids) may be fixed or cross-linked such that the molecules contained are immobilized within the cellular or subcellular component. In some embodiments, the sample (e.g., cells and subcellular components) is fixed such that the spatial location of the molecules within the sample are maintained.

In some cases, the sample undergoes fixation to crosslink proteins within the tissue or within a cellular structure and may stabilize the lipid membrane. In some examples, the sample is fixed using formaldehyde in phosphate buffered saline (PBS). Standard methods of fixation are known and include incubation with 0.5-5% formaldehyde in 1×PBS for 10-30 min. In some embodiments, the sample is fixed by incubation in methanol or ethanol. In some embodiments, after fixation, the sample is treated to permeabilized and allow access to the interior of the structural components by enzymes and DNA tags (e.g., recording tags, probe tags, spatial tags, or copies thereof, barcodes, or other nucleic acids).

In some embodiments, one or more washing steps are performed before and/or after fixation and/or permeabilization. Commercial fixation and permeabilization kits can be used to prepare the sample. In some embodiments, the fixing or cross-linking of the sample may be reversed.

In some embodiments, reversal of fixation or cross-linking of the sample is performed prior to isolating the macromolecules (e.g., proteins, polypeptides, or peptides) and associated recording tags from the spatial sample. In some embodiments, reversal of fixation or cross-linking of the sample is performed after isolating the macromolecules (e.g., proteins, polypeptides, or peptides) and associated recording tags from the spatial sample. For example, crosslinking may be reversed by incubating the cross-linked sample in high salt (approximately 200 mM NaCl) at 65° C. for about four hours or more.

In some embodiments, a tissue sample will be treated to remove embedding material (e.g. to remove paraffin or formalin) from the sample prior to release, capture or treatment of the macromolecules (e.g., proteins, polypeptides, or peptides) from the spatial sample. This can be achieved by contacting the sample with an appropriate solvent (e.g. xylene and ethanol washes). Treatment can occur prior to contacting the tissue sample with a solid support set forth herein or the treatment can occur while the tissue sample is on the solid support.

2. Providing a Recording Tag

The methods provided herein include providing a spatial sample comprising one or more macromolecules (e.g., proteins, polypeptides, or peptides) with a recording tag. In some embodiments, the spatial sample is provided with a plurality of recording tags. In some aspects, a plurality of macromolecules in a spatial sample is provided with recording tags. The recording tags may be associated or attached, directly or indirectly to the macromolecules or other moieties in the spatial sample. In some embodiments, the recording tags are attached to the macromolecules using any suitable means. In some embodiments, a macromolecule may be associated with one or more recording tags. In some aspects, the recording tag may be any suitable sequenceable moiety to which information from the probe tag, spatial tag, and optionally identifying information of one or more coding tags, can be transferred. The recording tag serves as a moiety to which information, such as information from the molecular probe or spatial probe, can be transferred or recorded.

In some other embodiments, the recording tags are not associated or attached, directly or indirectly to the macromolecules or other moieties in the spatial sample but are held in place in a matrix applied to the spatial sample. In some embodiments, the spatial sample is exposed to a matrix (e.g., a polymer matrix), scaffold, or other substance containing recording tags. See e.g., Gao et al., BMC Biology (2017) 15:50). For example, the matrix may comprise hydrogel polymer chains. In some embodiments, the spatial sample (e.g., a biological tissue or specimen) is chemically fixed and treated with compounds that bind to macromolecules such that the biomolecules are tethered to hydrogel polymer chains. For example, a hydrogel made of closely spaced, densely cross-linked, highly charged monomers is polymerized evenly throughout the cells or tissue in the spatial sample, intercalating between and around the macromolecules and biomolecules in the spatial sample. In some cases, the embedded spatial sample can be exposed to a mechanical homogenization step involving denaturation and/or digestion of structural molecules. In some embodiments, a spatial sample comprises a specimen-hydrogel composite.

In some embodiments of the provided methods, information from one or more probe tag, spatial tag, and/or coding tag is transferred to the recording tag. The recording tag may comprise other nucleic acid components. In some embodiments, the recording tag may comprise a unique molecular identifier, a compartment tag, a partition barcode, sample barcode, a fraction barcode, information transferred from a probe tag, information transferred from a spatial tag, a spacer sequence, a universal priming site, or any combination thereof. In some embodiments, the recording tag can further comprise other information including information from a macromolecule analysis assay, such as binder identifier (e.g., from a coding tag), cycle identifier (e.g., from a coding tag), etc.

In some embodiments, at least one recording tag is associated or co-localized directly or indirectly with the macromolecule (e.g., polypeptide). In a particular embodiment, a single recording tag is attached to a polypeptide, preferably via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

In some embodiments, the density or number of macromolecules provided with a recording tag is controlled or titrated. In other embodiments, the matrix or substance containing recording tags applied to the spatial sample is titrated for a desired density of recording tags. For example, it may be desirable to space the recording tags in or on the spatial sample appropriately to accommodate methods to be used to assess the spatial location of the macromolecules. In some cases, the amount or density of recording tags associated with macromolecules in the spatial sample is titrated on the surface of the sample or within the volume of the sample.

In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a macromolecule in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

A recording tag may comprise DNA, RNA, or polynucleotide analogs including PNA, γPNA, GNA, BNA, XNA, TNA, other polynucleotide analogs, or a combination thereof. A recording tag may be single stranded, or partially or completely double stranded. A recording tag may have a blunt end or overhanging end. A recording tag may comprise a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. In some embodiments, the recording tag may comprise a peptide or sequence of amino acids. In some cases, the recording tag is a moiety that allows a sequence of amino acids (e.g., a peptide barcode) to be attached or added.

In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of macromolecules within a sample are labeled with recording tags. In a particular embodiment, a subset of macromolecules from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the macromolecules in the spatial sample in situ. In some embodiments, the recording tags are attached to the macromolecules prior to providing the sample on a solid support. In some embodiments, the recording tags are attached to the macromolecules after providing the sample on the solid support.

In some embodiments, the recording tag can also include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a solid support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual macromolecules. In some embodiments, within a library of macromolecules, each macromolecule is associated with a single recording tag, with each recording tag comprising a unique UMI. In other embodiments, multiple copies of a recording tag are associated with a single macromolecule, with each copy of the recording tag comprising the same UMI. In some embodiments, a UMI has a different base sequence than the spacer or encoder sequences within the binding agents' coding tags to facilitate distinguishing these components during sequence analysis. In some embodiments, the UMI may provide function as a location identifier and also provide information in the macromolecule analysis assay. For example, the UMI may be used to identify molecules that are identical by descent, and therefore originated from the same initial molecule. In some aspects, this information can be used to correct for variations in amplification, and to detect and correct sequencing errors.

In some embodiments, the recording tag may comprise spatial information. For example, the recording tag may comprise a UMI which, in some cases, may serve as a spatial tag.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:1) or an Illumina P7 primer (5′-CAAGCAGAAGACGGCATACGAGAT-3′-SEQ ID NO:2).

The recording tags may comprise a reactive moiety for a cognate reactive moiety present on the target macromolecule, e.g., the target protein (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule. A suitable linker can be attached to various positions of the recording tag, such as the 3′ end, at an internal position, or within the linker attached to the 5′ end of the recording tag.

B. Molecular Probe

The methods provided herein include binding of one or more molecular probes to the spatial sample. In some embodiments, the molecular probe comprises a probe tag. After providing a spatial sample comprising one or more macromolecules with one or more recording tags, the method includes applying and binding one or more molecular probes to the spatial sample. In some embodiments, prior to binding of the spatial sample with one or more molecular probes, the spatial sample is treated with a blocking agent. The molecular probe may bind to a macromolecule in the spatial sample or a moiety in proximity to the macromolecule in the spatial sample.

In some embodiments, two or more molecular probes are applied to the spatial sample. In some cases where a plurality of molecular probes are used, molecular probes of the same identity may be associated with the same probe tag. The one or more molecular probes may be applied sequentially or a plurality of molecular probes may be applied at the same time. In some cases, the method may include decoding combinatorial information from transferring two or more probe tags serially to the recording tag. In some embodiments, a plurality of macromolecules and associated extended recording tags may contain the same barcode transferred from probe tags.

The molecular probe may be comprised of any composition suitable for binding the spatial sample. In some examples, the molecular probe comprises a nucleic acid, a peptide, a polypeptide, a protein, carbohydrate, or a small molecule that binds to, associates, unites with, recognizes, or combines with the spatial sample. The molecular probe may form a covalent association or non-covalent association with the spatial sample or a component of the spatial sample. In some aspects, the molecular probe may form a reversible association with the spatial sample or a component of the spatial sample. A molecular probe may be a chimeric molecule, composed of two or more types of molecules, such as a nucleic acid molecule-peptide chimeric molecular probe or a carbohydrate-peptide chimeric molecular probe. A molecular probe may be a naturally occurring, synthetically produced, or recombinantly expressed molecule. A molecular probe may bind to a linear molecule or a molecule having a three-dimensional structure (also referred to as conformation).

In some examples, the molecular probe comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

In some embodiments, the molecular probe comprises a microprotein (cysteine knot protein, knottin), a DARPin; a Tetranectin; an Affibody; an Affimer, a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)2, a peptide mimetic molecule, or a synthetic molecule (See e.g., Nelson, MAbs (2010) 2(1): 77-78, Goltsev et al., Cell. 2018 Aug. 9; 174(4):968-981, or as described in US Patent Nos. or Patent Publication Nos. U.S. Pat. Nos. 5,475,096, 5,831,012, 6,818,418, 7,166,697, 7,250,297, 7,417,130, 7,838,629, US 2004/0209243, and/or US 2010/0239633).

In some embodiments, the molecular probe is capable of chemically binding, covalently binding, and/or reversible binding to the spatial sample. In some embodiments, the molecular probe binds to a moiety that is bound to, associated with or complexed with the macromolecule in the spatial sample. In some examples, the molecular probe binds to a macromolecule (e.g., target macromolecule), a moiety in proximity to the macromolecule, or a moiety associated or bound to the macromolecule in the spatial sample. In some embodiments, the molecular probe binds a moiety in proximity to the macromolecule such that transfer of information from a probe tag can be transferred to a recording tag allow association with the molecular probe. For example, the distance between the macromolecule and the moiety in proximity to the macromolecule is about 10 nm to 100 nm; about 10 nm to 500 nm, about 10 nm to 1,000 nm, about 10 nm to 5,000 nm, about 100 nm to 300 nm; about 100 nm to 600 nm; about 100 nm to 1,000 nm; about 100 nm to 5,000 nm; about 300 nm to 600 nm, about 300 nm to 1,000 nm; or 300 nm to 5,000 nm. In some cases, transfer of information from the probe tag to the recording tag can occur if the recording tag is in proximity to the probe tag, regardless where the molecular probe is bound to the macromolecule. In some embodiments, the molecular probe is attached to the probe tag via a linker which may be of various lengths. In some cases, the length of the linker between the molecular probe and the probe tag may increase the distance between a moiety in proximity to the molecular probe and the molecular probe which allows association to the molecular probe. In some embodiments, the proximity of the moiety to the macromolecule may depend on the length of any linkers used in the molecular probe to attach the probe tag.

In some examples, the targeting moiety is configured to bind to a macromolecule, including but not limited to a nucleic acid, a carbohydrate, a lipid, a polypeptide, a post-translational modification of a polypeptide, or any combinations thereof. In some embodiments, the targeting moiety is a protein-specific targeting moiety, an epitope-specific targeting moiety, or a nucleic acid-specific targeting moiety. In some cases, the molecular probe is configured to bind to a cell surface marker. In some embodiments, the targeting moiety binds to a post-translational modifications (PTMs) of a polypeptide or amino acid. Examples of PTMs include but is not limited to phosphorylation, ubiquitination, methylation, acetylation, glycosylation, oxidation, lipidation, nitrosylation, SUMOylation, ubiquitination, and others.

In some embodiment, the molecular probe comprises a targeting moiety capable of specific or partially specific binding. In some embodiment, the molecular probe comprises a targeting moiety capable of specific and/or selective binding. An example of a structure-specific binder may include a protein-specific molecule that may bind to a protein target. Examples of suitable protein-specific molecules may include antibodies and antibody fragments, nucleic acids (for example, aptamers that recognize protein targets), or protein substrates. In some embodiments, a target of the targeting moiety may include an antigen and a molecular probe may include an antibody. A suitable antibody may include monoclonal antibodies, polyclonal antibodies, multi-specific antibodies (for example, bispecific antibodies), or antibody fragments so long as they bind specifically to a target antigen. In some embodiments, the molecular probe comprises a moiety or a nucleic acid component configured to specifically bind nucleic acids, such as a specific target nucleic acid sequence.

The molecular probes provided herein may optionally comprise a suitable detectable label, including but not limited to radioisotopes, fluorescent labels, colorimetric labels and various enzyme-substrate labels know in the art. In some embodiments, the signal from the detectable label can be amplified by binding a secondary probe to the primary molecular probe. For example, the secondary probe may be fluorescently labeled or may be conjugated to an enzyme that can then amplify a signal. In some embodiments, the detectable label or a secondary probe is detectable visually by microscopy or using an imager. In some embodiments, one or more steps of the method may be performed using an system, such as an automated system, including application of the molecular probes. In some embodiments, a microfluid system for cell analysis can be used which delivers and applies the reagents for the provided methods. In some aspects, the system for performing one or more steps of the method may be multiplex. For example, a multiplexed tissue processing platform may be utilized. In some embodiments, a microfluidic flow cell may be used for the binding of the molecular probes to the spatial sample.

In some embodiments, signal intensity, signal wavelength, signal location, signal frequency, or signal shift of the optional detectable label associated with the molecular probe is observed. In some embodiments, the observation of the detectable label may be performed prior to transfer of the information from the probe tag to the recording tag. In some cases, the observation of the detectable label may be performed after transfer of the information from the probe tag to the recording tag. In some embodiments, one or more aforementioned characteristics of the signal may be observed, measured, and recorded.

In the methods provided herein, the molecular probe comprises a probe tag comprising information to be transferred to the recording tag associated with the macromolecules (e.g., proteins, polypeptides, or peptides). In the methods provided herein, the molecular probe comprises a probe tag comprising information to be transferred to the recording tag contained in a matrix applied to the spatial sample. In some embodiments, the information from a plurality of probe tags is transferred to a plurality of recording tags. In some embodiments, the information from one probe tag is transferred to two or more recording tags. In some embodiments, the information from more than one probe tag is transferred to a recording tag. In some embodiments, the probe tag comprises a barcode. In some embodiments, the transferred information from the probe tag to the recording tag may also be referred to as a probe tag. In some aspects, the extended recording tag comprises a probe tag sequence.

In some embodiments, the use of the molecular probes may include adjustments useful for subsampling and/or tuning the dynamic range. In some cases, the concentration of molecular probes provided to the sample can be tuned and adjusted. For example, for detection of single molecules, the concertation of the molecular probes provided can be reduced. In some embodiments, the sample is provided with a plurality of molecule probes, wherein some molecular probes are labeled with a probe tag and some are not labeled with a probe tag (e.g. a “dummy molecular probe”). In some cases, the sample is provided with a plurality of molecular probes that includes at least about 10%, 15%, 20%, 25%, 30%, 35%, 40%, 45%, 50%, 55%, 60%, 65%, 70%, 75%, 80%, 85%, 90%, 95%, 97%, or 99% molecular probes that are not labeled with a probe tag (e.g. “dummy molecular probes”). In some aspects, the sample is provided with a plurality of molecule probes, wherein two or more of the same molecular probes are associated with different probe tags.

A plurality of macromolecules of the spatial sample can be labeled with a probe tag or contain information transferred from a probe tag comprising the same barcode. In some embodiments, a plurality of recording tags in proximity to probe tags associated with molecular probes can be extended by transferring information from the probe tags. The recording tags need not be attached or associated to the moiety bound by the molecular probe as long as the recording tags are in proximity to the probe tag. For example, the distance between the recording tag and the moiety or macromolecule bound by the molecular probe comprising the probe tag is about 10 nm to 100 nm; about 10 nm to 500 nm, about 10 nm to 1,000 nm, about 10 nm to 5,000 nm, about 100 nm to 300 nm; about 100 nm to 600 nm; about 100 nm to 1,000 nm; about 100 nm to 5,000 nm; about 300 nm to 600 nm, about 300 nm to 1,000 nm; or 300 nm to 5,000 nm. In some examples, a plurality of macromolecules within a cell may be labeled with a probe tag or contain information transferred from a probe tag comprising the same barcode. In some examples, a plurality of macromolecules within an organelle may be labeled with a probe tag or contain information transferred from a probe tag comprising the same barcode.

In some embodiments, a probe tag is a nucleic acid or an amino acid tag comprising a barcode that is transferred to the recording tag. In some cases, the recording tag may be associated with the macromolecules or be suspended in a matrix or substance applied to the spatial sample. In some embodiments, probe tag information is transferred to the recording tag by generating the sequence in situ on the recoding tag associated with the macromolecule in the spatial sample, thereby generating an extended recording tag. By transferring the information from the probe tag to the recording tag, in some embodiments, the extended recording tag comprises a probe tag. In some examples, the method includes generating in situ a sequence on the recording tag that contains a barcode sequence from the probe tag. In some embodiments, the probe tag is physically transferred to the recording tag. In some cases, extending the recording tag by transferring information from the probe tag associated with the molecular probe to the recording tag is performed using any suitable chemical/enzymatic reaction, such as ligation or polymerase extension. For example, ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof can be used to transfer information from the probe tag to the recording tag to generate an extended recording tag.

In certain embodiments, a probe tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length.

The probe tag may be any suitable tag. In some examples, the probe tag comprises a DNA molecule, DNA with pseudo-complementary bases, an RNA molecule, a BNA molecule, an XNA molecule, a LNA molecule, a PNA molecule, or a γPNA molecule. In some embodiments, the probe tag comprises a non-nucleic acid sequenceable polymer, e.g., a polysaccharide, a polypeptide, a peptide, or a polyamide, or a combination thereof. In some embodiments, the probe tag is a nucleic acid. In some embodiments, the probe tag comprises a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length. A probe tag may comprise a barcode sequence, which is optionally flanked by one spacer on one side or flanked by a spacer on each side. A probe tag may be single stranded or double stranded. A double stranded probe tag may comprise blunt ends, overhanging ends, or both. A probe tag may refer to the probe tag that is directly attached to a molecular probe, to a complementary sequence to the probe tag that is directly attached to a probe agent, or to probe tag information present in an extended recording tag.

In certain embodiments, a probe tag comprises a barcode. A barcode is a nucleic acid molecule of about 3 to about 30 bases, about 3 to about 25 bases, about 3 to about 20 bases, about 3 to about 10 bases, about 3 to about 10 bases, about 3 to about 8 bases in length. In some embodiments, a barcode is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In one embodiment, a barcode allows for multiplex sequencing of a plurality of samples or libraries. Barcodes can be used to de-convolute multiplexed sequence data and identify sequence reads from an individual sample or library. In some embodiments, the probe tag comprises more than one barcode. For example, the probe tag can be comprised of a string of 2 or more tags, each being a barcode. In some aspects, a concatenated string of barcodes can allow increased diversity of barcodes for labeling or identifying. For example, if 10 different tags (e.g., barcodes) are used and concatenated in a random way into a string of 3 tags as a barcode, then the concatenated barcode would have 10³=1000 possible sequences by using 10 tags arranged in a combinatorial manner. In some embodiments, a string of probe tags used in a combinatorial manner may be used to provide information regarding one or more molecular probes. For example, the recording tag may contain information in a series from one, two, three, four, five, six, seven, eight, nine, ten, or more probe tags.

In some embodiments, the probe tag comprises a spacer. In some embodiments, the spacer on the probe tag is configured to hybridize to a sequence comprised by the recording tag. In some cases, the probe tag comprises a spacer at the 5′ end. In some cases, the probe tag comprises a spacer at the 3′ end. In some embodiments, the probe tag comprises a universal priming site. In some embodiments, the probe tag further comprises other nucleic acid components. In some embodiments, the probe tag further comprises a universal priming site.

In some embodiments, the probe tag comprises a peptide or amino acid barcode, that comprises a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode that can be distinguished from other peptide barcodes can have different physical characteristics (amino acid sequence, sequence length, charge, size, molecular weight, hydrophobicity, reverse phase separation, affinity or other separable property). See e.g., International Patent Publication Nos. WO2016145416 and WO2018/078167. The probe tag may comprise a barcode that is associated with one molecular probe or a plurality of molecular probes. The molecular probes may be associated with or attached to the peptide barcode using any suitable means, including but not limited to any enzymatic or chemical attachment means. The information of the peptide barcode of the probe tag can be transferred to the recording tag using any suitable means, including but not limited to any enzymatic or chemical attachment means. See e.g., Miyamoto et al., PLoS One. (2019) 14(4):e0215993; Wroblewska et al., Cell. (2018) 175(4):1141-1155.e16. In some embodiments, linkers made of amino acid sequences that are typically flexible permitting the attachment of two different polypeptides can be used. For example, a linear linking peptide consists of between two and 25 amino acids, between two and 15 amino acids, or longer linkers can be used.

Information from the probe tag may be transferred to the recording tag in any suitable manner. In some embodiments, the method includes extending the recording tag by transferring information from one or more probe tags associated with the molecular probe to the recording tag. For example, information from the probe tag may be transferred to the recording tag by extension or ligation. In some embodiments, transferring information from the probe tag to the recording tag comprises contacting the spatial sample with a polymerase and a nucleotide mix, thereby adding one or more nucleotides to the recording tag. In some cases, the probe tag associated with the molecular probe serves as a template for extension. In certain embodiments, information of a probe tag is transferred to a recording tag via primer extension (see e.g., Chan et al., Curr Opin Chem Biol. (2015) 26: 55-61A spacer sequence on the terminus of a recording tag anneals with complementary spacer sequence on the opposite terminus of a probe tag and a polymerase (e.g., strand-displacing polymerase) extends the recording tag sequence, using the annealed probe tag as a template.

In some embodiments, information from the probe tag is capable of being transferred to any recording tag in proximity to the probe tag. The recording tags need not be attached or associated to the moiety bound by the molecular probe (either directly or indirectly) as long as the recording tags are in proximity to the probe tag for information transfer. The distance which allows the probe tag information to be transferred to the recording tag may depend on the distance a probe tag and recording tag may reach. For example, a molecular probe may be a nucleic acid that binds to a target nucleic acid and the target nucleic acid is bound to a polymerase. In this example, the polymerase is attached to a recording tag and the recording tag is in the vicinity of the probe tag attached to the target nucleic acid. In another example, a recording tag contained in a matrix applied to the spatial sample may be in proximity to a probe tag attached to a molecular probe that is bound to a polypeptide in the spatial sample.

The transferring of information from the probe tag to a recording tag can be directly from the probe tag associated with the molecular probe or indirectly via a copy of the probe tag. In some embodiments, the probe tag associated with the molecular probe is copied one or more times prior to transferring the information of the probe tag to a recording tag. For example, the probe tag associated with the molecular probe may be amplified before transferring the information of the probe tag to a recording tag. In some cases, the amplification of the probe tag is linear amplification. In some aspects, the amplification of the probe tag is performed using a RNA polymerase. In cases where copies of the probe tag comprises RNA, the transferring of the probe tag to the recording tag may be performed using reverse transcription. In one example, the molecular probe may bind to a cell surface marker and recording tags are inside a cell. In this case, copies of the probe tag attached to the molecular probe bound to the outside of the cell is made, and the copies of the probe tag may then diffuse into the cells and transfer of information from the copies of the probe tag to the recording tags inside the cells may occur.

C. Spatial Probe

The methods provided herein include binding of one or more spatial probes to the spatial sample. In some embodiments, the spatial probe comprises a spatial tag. In some cases, the spatial tag may comprise one or more nucleic acid components, including a barcode and optionally a spacer and/or universal priming site. After providing a spatial sample comprising one or more macromolecules with one or more recording tags, the method includes providing one or more spatial probes to the spatial sample. In some examples, the method includes providing a plurality of spatial probes to the spatial sample. In some embodiments, information from the spatial probe is transferred to the recording tag, thereby generating an extended recording tag. In some embodiments, the method include performing steps (b1) providing a spatial probe comprising a spatial tag to the spatial sample; (b2) determining the spatial tag in situ to obtain the spatial location of the spatial tag in the spatial sample; and (b3) extending the recording tag by transferring information from the spatial tag associated with the spatial probe to the recording tag are performed. In some embodiments, information (e.g., barcode) from the spatial tag is capable of being transferred to any recording tag in proximity to the spatial probe.

Exemplary steps involving the spatial probes may include: providing a plurality of polypeptides with spatial probes comprising spatial tags; attaching DNA barcodes to beads via a photocleavable, chemical, or enzymatic linker which enables removal and subsequent diffusive transfer of the barcodes to the tissue section; providing barcoded beads to the spatial sample which may attach to or associate non-specifically with the tissue surface through adhesive forces such as charge interaction, DNA hybridization, or reversible chemical coupling; decoding or sequencing the tissue-attached barcoded DNA beads; releasing DNA barcodes by enzymatic, chemical, or photocleavage of the cleavable linker; allowing barcodes to permeate the tissue slice and anneal to the DNA recording tags attached to macromolecules, e.g., proteins within the tissue slice; and performing a reaction (e.g., polymerase extension) to transfer the barcodes to the recording tags on the macromolecules in the spatial sample. In some embodiments, the barcoded beads may be provided in any suitable formats, including any described herein.

In some embodiments, the spatial probe comprises a nucleic acid, a support, a polypeptide, a small molecule, and/or a chemical moiety. In some embodiments, the spatial probe comprises a support, e.g., a solid support, and a spatial tag comprising a nucleic acid. In some preferred embodiments, the spatial probe contains a support attached to a plurality of nucleic acids (e.g., spatial tag). For example, the support is a bead or a microparticle. Any suitable bead material and size may be used to deliver barcodes to the polypeptides in the sample, including but not limited to porous or non-solid beads. In some embodiments, the spatial probe comprises a barcoded bead. In some examples, the beads are porous to accommodate a higher loading of barcodes on a bead. In some cases, the spatial probe comprises two or more copies of the same barcodes. In some embodiment, the bead is a polystyrene bead, a polyacrylate bead, a cellulose bead, a dextran bead, a polymer bead, an agarose bead, an acrylamide bead, a solid core bead, a porous bead, a paramagnetic bead, glass bead, or a controlled pore bead, or any combinations thereof.

In some embodiments, the spatial sample labeled by a spatial tag from a spatial probe is determined by the size of the spatial probe. For example, a single molecule or a plurality of molecules in a region may be labeled with spatial tags from a spatial probe. In some aspects, the size of the spatial probe may be selected and adjusted based on the resolution preferred. Other characteristics of the spatial probe may also be considered including packing, stability, layering, etc. In some embodiments, the spatial probe size or type is selected based on the ability to optically resolve the probes, e.g., imaging resolution or sensor resolution. In some examples, the spatial probe (e.g., bead or nanoparticle) ranges between about 50 nm to about 10 μm, between about 50 nm to about 1 μm, between about 50 nm to about 100 nm, between about 100 nm to about 1 μm, between about 100 nm to about 10 μm, between about 0.1 μm to about 100 μm, between about 0.1 μm to about 50 μm, between about 10 μm to about 50 μm, between about 5 μm to about 10 μm, between about 0.5 μm to about 100 μm, between about 0.5 μm to about 50 μm, between about 0.5 μm to about 10 μm, between about 0.5 μm to about 5 μm, or between about 0.5 μm to about 1 μm in diameter. In some examples, the beads are about 50 nm to about 10 μm in diameter.

In some embodiments, the probe comprises one or more spatial tags attached to the support with a cleavable linker. In some embodiments, DNA barcodes are attached to beads via a photocleavable, chemical, or enzymatic linker which enables removal and subsequent diffusive transfer of the barcodes to the tissue section. DNA barcodes may be released by enzymatic, chemical, or photocleavage of a cleavable linker. Various methods can be used to generate the barcoded beads and apply to the sample, including a split-pool synthesis strategy as described in Klein et al., Lab Chip (2017) 17(15): 2540-2541; covering a surface with DNA-barcoded beads as described in Rodrigues et al., Science (2019) 363(6434):1463-1467; or use of a spatially barcoded bead array as described in Vickovic et al. (2019) Nat Methods 16(10): 987-990. For example, use of spatially indexed beads can include distributing beads on a planar surface and barcoding positions correlated with spatial position. In some aspects, each bead has a single population of DNA barcodes. DNA barcodes are attached to the bead using any suitable methods. In some cases, the spatial tag (e.g., barcodes) are cleaved from the beads and transferred to the polypeptides. In some embodiments, the cleavage of the barcode from the bead is via photocleavage such as by exposure to long wavelength UV. The cleaved barcodes diffuse into the tissue section of the spatial sample and hybridize to recording tags. The released barcodes may be transferred to the recording tags using any suitable methods, including but not limited to by ligation or extension. For example, ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof can be used to transfer information from the spatial tag to the recording tag to generate an extended recording tag. In some embodiments, a polymerase extension mix is added to the spatial sample to transfer barcode information from the hybridized barcode to the DNA recording tag.

In some embodiments, the spatial tag is assessed in situ in the spatial sample or after associating with macromolecules in the spatial sample. For example, randomly distributed barcodes are provided to the spatial sample and the barcodes are decoded or assessed in situ. In some embodiments, the barcodes can be decoded or assessed in situ before or after transferring to the recording tag. In some embodiments, the barcodes can be decoded or assessed in situ after it is in the spatial location and position for transfer to the surface where the spatial sample is immobilized. For example, the barcodes of the spatial tag can be decoded or assessed while attached to the spatial probe or after being transferred to the recording tag. In some embodiments, the barcodes are not known prior to being decoded or assessed in situ. In some aspects, the assessing of the spatial tag is prior to releasing macromolecules of the sample for further macromolecule analysis.

In one example, barcoded beads form an array which are spatially indexed prior to transferring the barcodes to the polypeptides (See e.g., Rodrigues et al., Science (2019) 363(6434):1463-1467). In some cases, the method includes determining the spatial tag in situ to obtain the spatial location of the spatial tag in the spatial sample. In some embodiments, determining the spatial tag in situ to obtain the spatial location of the spatial tag in the spatial sample is performed while the spatial tag is attached to a support. In some embodiments, determining the spatial tag in situ to obtain the spatial location of the spatial tag in the spatial sample is performed after the spatial tag is released or cleaved from the support.

In some other embodiments, the spatial sample is labeled with barcodes reflecting the spatial position of the molecule within the cellular tissue mounted on a surface, then the spatial distribution of protein analytes within the tissue slice can later be reconstructed after sequence analysis, much as is done for spatial transcriptomics (e.g., Stahl et al. 2016 Science 353(6294):78-82; Crosetto et al. Nat Rev Genet. 2015 January; 16(1):57-66). In another embodiment, molecules in cellular organelles and cellular/subcellular compartments can be labeled (Christoforou et al., 2016, Nat. Commun. 7:8992; Lundberg et al., (2019) Nat Rev Mol Cell Biol 20(5): 285-302, incorporated by reference in its entirety). A number of approaches can be used to provide intracellular barcodes to attach to proximal proteins. Some methods of spatial cellular labelling are described in the review by Marx, 2015, Nat Methods 12:815-819, incorporated by reference in its entirety.

In one embodiment, the macromolecules (e.g. polypeptides) in the spatial sample are provided with a recording tag which comprises a sequence of nucleotides that is complementary to at least a portion of the spatial tag or a portion thereof. In some embodiments, the spatial tag comprises a barcode and a sequence of nucleotides complementary to the recording tag. In some embodiments, the complementary sequence shared by the recoding tag and spatial tag is useful for transferring a barcode from the spatial tag to the recording tag. In some cases, the complementary sequence allows association between the barcode from the spatial tag and the recording tag. In some embodiments for providing and transferring a spatial tag to a recording tag attached to polypeptides, the barcode on the bead is flanked by an upstream spacer sequence and a downstream primer extension sequence complementary to the at least a portion of the recording tag attached to the polypeptides.

The spatial tag may be any suitable tag. In some examples, the spatial tag comprises a DNA molecule, DNA with pseudo-complementary bases, an RNA molecule, a BNA molecule, an XNA molecule, a LNA molecule, a PNA molecule, or a γPNA molecule. In some embodiments, the spatial tag comprises a non-nucleic acid sequenceable polymer, e.g., a polysaccharide, a polypeptide, a peptide, or a polyamide, or a combination thereof. In some embodiments, the spatial tag is a nucleic acid. In some embodiments, the spatial tag comprises a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length. A spatial tag may comprise a barcode sequence, which is optionally flanked by one spacer on one side or flanked by a spacer on each side. A spatial tag may be single stranded or double stranded. A double stranded spatial tag may comprise blunt ends, overhanging ends, or both. A spatial tag may refer to the spatial tag that is associated with the spatial probe (e.g., a bead), to a complementary sequence to the spatial tag that is directly attached to associated with the spatial probe (e.g., a bead), or to spatial tag information present in an extended recording tag.

In certain embodiments, a spatial tag comprises a barcode. See e.g. Weinstein et al., Cell. 2019 Jun. 27; 178(1):229-241. A barcode is a nucleic acid molecule of about 3 to about 30 bases, about 3 to about 25 bases, about 3 to about 20 bases, about 3 to about 10 bases, about 3 to about 10 bases, about 3 to about 8 bases in length. In some embodiments, a barcode is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In one embodiment, a barcode allows for multiplex sequencing of a plurality of samples or libraries. Barcodes can be used to de-convolute multiplexed sequence data and identify sequence reads from an individual sample or library. In some embodiments, the spatial tag comprises more than one barcode. For example, the spatial tag can be comprised of a string of 2 or more tags, each being a barcode. In some aspects, a concatenated string of barcodes can allow increased diversity of barcodes for labeling or identifying. In some embodiments, a string of spatial tags used in a combinatorial manner may be used to provide information regarding one or more molecular probes.

In certain embodiments, a spatial tag comprises an optional, unique molecular identifier (UMI), which provides a unique identifier tag for each macromolecule (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length.

In some embodiments, the spatial tag comprises a spacer. In some embodiments, the spacer on the spatial tag is configured to hybridize to a sequence comprised by the recording tag. In some cases, the spatial tag comprises a spacer at the 5′ end. In some cases, the spatial tag comprises a spacer at the 3′ end. In some embodiments, the spatial tag comprises a universal priming site. In some embodiments, the spatial tag further comprises other nucleic acid components. In some embodiments, the spatial tag further comprises a universal priming site.

In some embodiments, the spatial tags (e.g., barcodes) are transferred from a solid substrate to the sample using various ways. For example, the barcodes are transferred from microparticles (e.g., beads) to the macromolecules in the sample. In some examples, a tissue sample on a surface is exposed to a plurality of beads with barcodes attached and the barcodes are transferred to the macromolecules (e.g. polypeptides). Each bead may contain multiple barcodes with the same sequence. In some examples, the barcodes from the barcoded beads are randomly attached to the macromolecules of the spatial sample. In some embodiments, the beads are delivered to the spatial sample by embedding the barcoded beads in a hydrogel coated over the tissue section surface. In some embodiments, a capillary gap flow cell may be used to deliver or distribute barcoded beads to the spatial sample.

In some embodiments, the spatial tag comprises a peptide or amino acid barcode, that comprises a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode that can be distinguished from other peptide barcodes can have different physical characteristics (amino acid sequence, sequence length, charge, size, molecular weight, hydrophobicity, reverse phase separation, affinity or other separable property). See e.g., International Patent Publication Nos. WO2016145416 and WO2018/078167. The spatial probe may be associated with or attached to the peptide barcode using any suitable means, including but not limited to any enzymatic or chemical attachment means. The information of the peptide barcode of the spatial tag can be transferred to the recording tag using any suitable means, including but not limited to any enzymatic or chemical attachment means. See e.g., Miyamoto et al., PLoS One. (2019) 14(4):e0215993; Wroblewska et al., Cell. (2018)175(4):1141-1155.e16. In some embodiments, linkers made of amino acid sequences that are typically flexible permitting the attachment of two different polypeptides can be used. For example, a linear linking peptide consists of between two and 25 amino acids, between two and 15 amino acids, or longer linkers can be used.

In other embodiments, the method includes a step in which the barcodes are assessed, determined, detected and/or analyzed in situ. In some cases, the barcodes are analyzed, decoded and/or sequenced in situ after the barcodes are randomly transferred to the spatial sample. For example, the spatial tags attached to the spatial probe (e.g., bead) are determined in situ to provide information of the spatial location of the spatial tag in the sample. In this case, the spatial tags are assessed before being released from beads. In other examples, the barcodes can be determined after the barcodes are released from the beads. Spatial decoding of the barcoded beads on the tissue sample may be performed before the barcodes are attached to the recording tags. The assembled barcoded beads may be spatially decoded in situ using fluorescent imaging and combinatorial hybridization-based approaches or in situ NGS sequencing (See e.g., Gunderson et al., Genome Res (2004) 14(5): 870-877; Lee et al., Nat Protoc. (2015) 10(3): 442-458, Rodrigues et al., Science (2019) 363(6434): 1463-1467); Goltsev et al., Cell. 2018 Aug. 9; 174(4):968-981; U.S. Patent Application Publication No. US 2014/0066318). In some embodiments, the decoding of barcoded beads is performed to generate sequences containing information of location in the spatial sample as described herein.

The transfer of the barcodes from the bead to the polypeptides may utilize any suitable methods, such as transfer by enzymatic means, including ligation or extension. In some cases, extending the recording tag by transferring information from the spatial to the recording tag is performed using any suitable chemical/enzymatic reaction, such as ligation or polymerase extension. For example, ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof can be used. In some embodiments, the beads are released after transfer of the barcode to the recording tags.

In some embodiments, determining the spatial tag to obtain the spatial location of the spatial tag in the spatial sample is performed in situ. For example, determining the spatial tag in situ is performed using a microscope based method. In some cases, determining the spatial tag in situ is performed using a fluorescence based method. In some cases, determining the spatial tag in situ is performed using a multiplex microscope and/or fluorescence based method. In some embodiments, determining the spatial tag in situ generates a visual signal. In some embodiments, the methods includes in situ sequencing or labeling of the protein. In some examples, determining the spatial tag in situ provides position information of the spatial tag (e.g., spatial position information in reference to the spatial sample). For single molecule decoding, hybridization of several rounds of pooled fluorescently-labeled decoding oligonucleotides can be used (See e.g., Gunderson et al., Genome Res (2004) 14(5): 870-877). In some embodiments, determining the spatial tag in situ comprises using one or more decoders, wherein the decoder comprises one or more detectable labels and a sequence complementary to the spatial tag or a portion thereof. In some examples, the detectable label comprises a radioisotope, a fluorescent label, a colorimetric label, or an enzyme-substrate label. For example, two or more decoders are used to detect one or more of the spatial tags.

In some embodiments, determining the spatial tag in situ to obtain the spatial location of the spatial tag in the spatial sample is performed using sequencing methods including, but not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

In some of any such embodiments, the method includes use of any microscopy methods know in the art and as described here. For example, fluorescently-labeled decoding oligonucleotides may be imaged. More than one image may be obtained. In some embodiments, the method includes correlating spatial location of the spatial tag with barcode sequences of the spatial tag.

### III. ANALYZING MACROMOLECULES USING MOLECULAR PROBES WITH A DETECTABLE LABEL

Provided herein are methods for analyzing a macromolecule (e.g., polypeptide or polynucleotide) comprising (a) providing a spatial sample comprising a macromolecule with a recording tag; (b) binding a molecular probe comprising a detectable label and a probe tag to the macromolecule or a moiety in proximity to the macromolecule in the spatial sample; (c) transferring information from the probe tag in the molecular probe to the recording tag to generate an extended recording tag; (d) assessing, e.g., observing, the detectable label to obtain spatial information of the molecular probe; (e) determining at least the sequence of the probe tag in the extended recording tag; and (f) correlating the sequence of the probe tag determined in step (e) with the molecular probe; thereby associating information from the sequence determined in step (e) with its spatial information determined in step (d).

Provided herein are methods for analyzing a macromolecule (e.g., polypeptide or polynucleotide) comprising providing a spatial sample comprising a macromolecule with a recording tag; binding a molecular probe comprising a detectable label and a probe tag to the spatial sample, such as by binding to the macromolecule or a moiety in proximity to the macromolecule in the spatial sample; transferring information from the probe tag associated in molecular probe to the recording tag to generate an extended recording tag; and assessing, e.g., observing, the detectable label to obtain spatial information of the molecular probe. The steps including binding a molecular probe to the sample, transferring information from the probe tag, and assessing, e.g., observing, the detectable label can be repeated one or more times. In some embodiments, the method further includes determining the sequence of the extended recording tag which includes one or more probe tags. In some aspects, the sequence of a series of probe tags (e.g., barcodes) is correlated with the molecular probes bound to the sample. In some embodiments, the information of the molecular probe(s), including target of the molecular probe(s) and other characteristics of the macromolecule bound by the molecular probe(s) can be associated with the spatial information from assessing, e.g., observing, the detectable label associated with the molecular probe. In some embodiments, the sample is sequentially bound by two or more molecular probes. In some cases, the molecular probe is removed, or the detectable label is inactivated after the detectable label has been observed.

In some embodiments, the macromolecule is a polypeptide. In some examples, the macromolecule analysis assay comprises a polypeptide analysis assay.

Some of the steps of the provided methods may be reversed or performed in various orders. In some embodiments, the macromolecule analysis assay is not performed. In some examples, steps (a), (b), (c), (d), (e), and (f) occur in sequential order. In other examples, steps (a), (b), (d), (c), (e), and (f) occur in sequential order. In some examples, steps (a), (b), (c), (d), (e), and (f) occur in sequential order. In some examples, steps (a), (b), (d), (c), (e), and (f) occur in sequential order. In some cases, one or more steps of the method is repeated. In some embodiments, step (d) is repeated two or more times. In some cases, the method includes repeating step (b) and step (c) sequentially two or more times. In some examples, the method includes removing the molecular probe from the spatial sample prior to repeating step (b). In some cases, the assessing, e.g., observing, of the detectable label is repeated for methods involving the binding of two or more molecular probes. In some embodiments, steps (b), (c), and (d) are sequentially repeated two or more times prior to performing steps (e) and (f). In some cases, steps (b), (c), and (d) are sequentially repeated two or more times prior to performing a macromolecule analysis assay. In some embodiments, steps (b), (d), and (c) are sequentially repeated two or more times prior to performing steps (e) and (f). In some cases, steps (b), (d), and (c) are sequentially repeated two or more times prior to performing a macromolecule analysis assay. In methods including performing a macromolecule analysis assay, the assay can be performed after steps (a), (b), (c), and (d). In methods including performing a macromolecule analysis assay, the assay can be performed prior to steps (e) and (f).

In some embodiments, the extended recording tag analyzed comprises information from a plurality of probe tags sequentially transferred to the recording tag. In some embodiments, the extended recording tag comprises information from one or more probe tags and one or more coding tags. In some cases, the extended recording tag comprises information from two or more probe tags and two or more coding tags. In some embodiments, the recording tag (e.g., extended recording tag) is directly or indirectly attached to the macromolecule. In some embodiments, the extended recording tag is not attached to the macromolecule.

In some embodiments of the provided methods, the molecular probe binds to the spatial sample by binding to a macromolecule in the spatial sample. In some embodiments of the provided methods, the molecular probe binds to the spatial sample by binding to a moiety in proximity to the macromolecule in the spatial sample. In some embodiments, a plurality of molecular probes is applied to the spatial sample. In some embodiments, the molecular probe is capable of selective and/or specific binding. In some embodiments, the molecular probe binds to a macromolecule in complex with other macromolecules. For example, the molecular probe may bind to a nucleic acid in a complex with a polypeptide of interest. In some specific embodiments, the molecular probe binds to the polypeptide to which the recording tag is associated or attached. In some specific embodiments, the molecular probe binds to a macromolecule and the binding brings the probe tag in the molecular probe into proximity to a recording tag applied to the spatial sample.

The molecular probe comprises a probe tag which may comprise any sequenceable molecule. In some examples, the probe tag comprises a barcode. The information of the probe tag is transferred in any suitable manner to the recording tag. In some aspects, the transferred information from one or more probe tags to a particular recording tag links the information from the one or more molecular probes to spatial information of the molecular probe(s) and the bound location. In some embodiments, the information from one probe tag may be transferred to two or more recording tags. In some embodiments, the information from two or more probe tags may be transferred to one recording tag.

In some embodiments, a spatial sample includes a biological sample. For example, the spatial sample may include macromolecules, cells, and/or tissues obtained from a subject. In some examples, the spatial sample is derived from a sample such as an intact tissue or a liquid sample. For example, the liquid sample may be spread deposited onto a surface prior to performing the methods. In some examples, the spatial sample is processed prior to binding of the molecular probes to the spatial sample, such as by treating the sample with a permeabilizing, fixing, and/or cross-linking reagent. In some embodiments, the spatial sample is exposed to a matrix or other substance containing recording tags. For example, the matrix may comprise hydrogel polymer chains.

In some embodiments, the method include further performing a macromolecule (e.g., polypeptide or polynucleotide) analysis assay in situ. In some other embodiments, the macromolecule analysis assay is performed after the macromolecules are released from the spatial sample. In some embodiments including additionally performing a macromolecule analysis assay, the macromolecule is attached to or associated with one or more recording tags. In some of any such embodiments, the macromolecule analysis assay includes one or more cycles of contacting the macromolecule with a binding agent capable of binding to the macromolecule, wherein the binding agent comprises a coding tag with identifying information regarding the binding agent; and transferring the information of the coding tag to the recording tag to extend to the recording tag. The identifying information from the binding agent is transferred to the recording tag associated with the polypeptide which also comprises information transferred from the probe tag. Thus, in some embodiments, the extended recording tag comprises information from one or more probe tags, and optionally one or more coding tag. In some embodiments, the method further includes determining at least a portion of the sequence of the macromolecule or the identity of the macromolecule and associating with the spatial location of the molecular probe determined in step (d).

In some embodiments, the macromolecule analysis assay comprises determining the sequence of at least a portion of a macromolecule (e.g., polypeptide or polynucleotide). In some cases, the analysis method may include performing any of the methods as described in International Patent Publication No. WO 2017/192633. In some cases, the sequence of a polypeptide is analyzed by construction of an extended nucleic acid sequence which represents the polypeptide sequence or a portion thereof, such as an extended nucleic acid onto the recording tag (or any additional barcodes or tags attached thereto).

An exemplary workflow for analyzing polypeptides may include the following: a spatial sample is provided on a solid support. The polypeptides of the spatial sample are labeled with recording tags or the spatial sample is exposed to a matrix containing recording tags. The recording tags may include a universal priming site that is useful for later amplification. A plurality of molecular probes each comprising a detectable label and a probe tag is applied to the spatial sample and binds to the sample. The information from the probe tags are transferred to recording tags by a suitable method, such as by ligation or extension. After transfer of the information from the probe tags, the molecular probes may be removed, released, or washed. Optionally, additional rounds of binding with molecular probes and transferring information from the probe tags to the recording tags may be performed. The detectable labels of the molecular probe is assessed and/or observed, such as by using imaging. In some embodiments where multiple cycles of binding with molecular probes are performed, the observation of the detectable label may include more than one imaging step. After assessing or observing the detectable label, the recording tags may be released and collected for analysis, such as for sequencing. If a macromolecule analysis assay is further performed, after transfer of information from the probe tag, polypeptides attached to recording tags are used and released from the spatial sample. In an optional step, the polypeptides are digested. Prior to performing the polypeptide analysis assay, the polypeptides and associated recording tags (comprising information from the probe tags) can be immobilized randomly on a single molecule sequencing substrate (e.g., beads) at an appropriate intramolecular spacing. A polypeptide analysis assay is performed on the polypeptides associated with the recording tag, thereby further adding information to the extended recording tags. At least a portion of the sequence of the extended recording tag (with the information from the probe tag) is determined. The sequence of the information from the probe tag determined from the extended recording tag is correlated with the molecular probe associated with the same probe tag; thereby associating information from the sequence determined from the extended recording tag with its spatial information determined from assessing, e.g., observing, the detectable label associated with the molecular probe. Any information regarding the sample bound by the molecular probe may also be correlated with the spatial information including tissue/cell phenotype, state, and presence or absence of particular markers. Using this workflow, the information in the extended recording tag is associated with spatial location of the molecular probe.

A. Samples

In one aspect, the present disclosure relates to the analysis of macromolecules from a sample. A macromolecule can be a large molecule composed of smaller subunits. In certain embodiments, a macromolecule is a protein, a protein complex, polypeptide, peptide, nucleic acid molecule, carbohydrate, lipid, macrocycle, or a chimeric macromolecule. In some embodiments, the macromolecule is a protein, a polypeptide, or a peptide.

In some embodiments, the macromolecules (e.g., proteins, polypeptides, or peptides) are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. The sample may be a spatial sample, from which information regarding the spatial arrangement and/or location of anatomical features, morphological features, cellular features, and/or subcellular features may be desired. In some embodiments, the sample is further processed by methods known in the art. For example, a sample is processed to remove, clear, or isolate cellular material (e.g., by centrifugation, filtration, etc.). The spatial sample may refer to a biological sample arranged such that constituents, portions, or regions of the sample may be referenced spatially (e.g., arranged in a planar format such as a tissue section on a slide). In some embodiments, the sample comprises two or more cells.

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids. In some of any of the provided embodiments, a sample, such as a fluid sample, may be deposited on a surface. For example, a liquid sample may be processed to prepare a cell spread on a solid surface such as a slide. In some embodiments, a sample or a portion thereof (such as analytes or cells obtained from the sample) may be deposited in a polymer resin. In some cases, the polymer resin comprises a hydrogel-forming natural or synthetic polymer.

In some embodiments, the sample is a tissue sample. A tissue can be prepared in any convenient or desired way for its use in any of the methods described herein. Fresh, frozen, fixed or unfixed tissues can be used. A tissue can be prepared, fixed or embedded using methods described herein or known in the art (Fischer et al., CSH Protoc (2008) pdb prot4991; Fischer et al., CSH Protoc (2008) pdb top36; Fischer et al., CSH Protoc. (2008) pdb.prot4988). The tissue can be freshly excised from an organism or it may have been previously preserved for example by freezing, embedding in a material such as paraffin (e.g. formalin fixed paraffin embedded samples), formalin fixation, infiltration, dehydration or the like. In some examples, a matrix-forming material can be used to encapsulate a biological sample, such as a tissue sample. In some cases, the sample is embedded in a paraffin block. For example, the spatial sample may be a formalin-fixed, paraffin-embedded (FFPE) section. Optionally, a tissue section can be attached to a solid support, for example, using techniques and compositions exemplified herein with regard to attaching nucleic acids, cells, viruses, beads or the like to a solid support (Ramos-Vera et al., J Vet Diagn Invest. (2008) 20(4):393-413). As a further option, a tissue can be permeabilized and the cells of the tissue lysed when the tissue is in contact with a solid support. Standard conditions and reagents may be used for tissue permeabilization including incubation with any suitable detergents, Triton X-100, ethoxylated nonylphenol (Tergitol-type NP-40), Tween 20, Saponin, Digitonin, or acetone (Fischer et al., CSH Protoc (2008) pdb top36).

In some embodiments, the sample is a “planar sample” that is substantially planar, i.e., two dimensional. In some embodiments, a sample is deposited in a substrate or deposited on a solid surface. In some embodiments, the sample is a three dimensional sample. In some examples, a material or substrate (e.g. glass, metal, ceramics, organic polymer surface or gel) may contain cells or any combination of biomolecules derived from cells, such as proteins, nucleic acids, lipids, oligo/polysaccharides, biomolecule complexes, cellular organelles, extracellular vesicles (exosomes, micro vesicles), cellular debris or excretions. In some embodiments, the planar cellular sample can be made by, e.g., depositing cells or portions thereof on a planar surface, e.g., by centrifugation, by cutting a three dimensional object that contains cells into sections and mounting the sections onto a planar surface, i.e., producing a tissue section. In some embodiments, the sample is a tissue section that refers to a piece of tissue that has been obtained from a subject, fixed, sectioned (e.g., cryosectioning), and mounted on a planar surface, e.g., a microscope slide.

In some embodiments, the spatial sample (e.g., specimen or tissue sample) is treated to expand the sample. In some aspects, the spatial sample is preserved and expanded isotropically using a chemical process. For example, a tissue sample may be treated to attach anchors to biomolecules in the spatial sample, perform in situ polymer synthesis, perform mechanical homogenization, and perform specimen expansion (See e.g., Zhao et al., Nature Biotechnology (2017) 35(8):757-764; Chang et al., Nature Methods (2017) 14:593-599; Chang et al., Nature Methods (2016) 13(8):679-84; Tillberg et al., Nature Biotechnology (2016) 34:987-992; Chen et al., Science (2015) 347(6221):543-548; Asano et al., Current Protocols in Cell Biology (2018) 80(1):e56; Wassie et al., Nature Methods (2018) 16(1):33-41; Boyden et al., Mater. Horiz., (2019) 6, 11-13; Alon et al., FEB S J. 2019 April; 286(8):1482-1494. Karagiannis et al., Current Opinion in Neurobiology (2018) 50:56-63; Gao et al., BMC Biology (2017) 15:50).

In some embodiments, the method includes obtaining and preparing macromolecules (e.g., polypeptides and proteins) from a single cell type or multiple cell types. In some embodiments, the sample comprises a population of cells. In some embodiments, the macromolecules (e.g., proteins, polypeptides, or peptides) are from a cellular or subcellular component, an extracellular vesicle, an organelle, or an organized subcomponent thereof. In some embodiments, the polypeptides are from one or more packaging of molecules (e.g., separate components of a single cell or separate components isolated from a population of cells, such as organelles or vesicles). The macromolecules (e.g., proteins, polypeptides, or peptides) may be from organelles, for example, mitochondria, nuclei, or cellular vesicles. In one embodiment, one or more specific types of single cells or subtypes thereof may be isolated. In some embodiments, the spatial samples may include but are not limited to cellular organelles, (e.g., nucleus, golgi apparatus, ribosomes, mitochondria, endoplasmic reticulum, chloroplast, cell membrane, vesicles, etc.).

1. Fixation and Permeabilization

In some embodiments, the methods provided herein further include one or more fixing (e.g., cross linking) and/or permeabilizing steps. In certain embodiments, the sample comprising macromolecules (e.g., proteins, polypeptides, or peptides) for analysis may be fixed and/or permeabilized. In some embodiments, the fixing, cross-linking, and/or permeabilizing the spatial sample is performed prior to providing the spatial sample with a recording tag. In some embodiments, the fixing, cross-linking, and/or permeabilizing the spatial sample is performed prior to binding a molecular probe to the macromolecule or a moiety in proximity to the macromolecule in the spatial sample. For example, holes or openings may be formed in membranes of the cells and/or any subcellular components. The cells, subcellular structures and components, or biomolecules may be fixed using any number of reagents including but not limited to formalin, methanol, ethanol, paraformaldehyde, formaldehyde, methanol: acetic acid, glutaraldehyde, bifunctional crosslinkers such as bis(succinimidyl)suberate, bis(succinimidyl)polyethyleneglycole etc.

In some examples, the methods of treating proteins and analyzing proteins provided herein may comprise fixing the sample at any step in the analysis method. In some cases, fixing the sample is performed prior to permeabilizing the sample (e.g., permeabilizing the cells or other membranes). In some examples, fixing the sample is performed after permeabilizing the sample. In some embodiments, the sample is fixed or cross linked prior to providing a protein in a spatial sample with a recording tag. In some embodiments, the sample is permeabilized prior to binding the spatial sample with one or more molecular probes.

In some embodiments, the samples may be fixed or cross-linked such that the cellular and subcellular components are immobilized or held in place. In some embodiments, the macromolecules in the sample (e.g., DNA, RNA, proteins, polypeptides, lipids) may be fixed or cross-linked such that the molecules contained are immobilized within the cellular or subcellular component. In some embodiments, the sample (e.g., cells and subcellular components) is fixed such that the spatial location of the molecules within the sample are maintained.

In some cases, the sample undergoes fixation to crosslink proteins within the tissue or within a cellular structure and may stabilize the lipid membrane. In some examples, the sample is fixed using formaldehyde in phosphate buffered saline (PBS). Standard methods of fixation are known and include incubation with 0.5-5% formaldehyde in 1×PBS for 10-30 min. In some embodiments, the sample is fixed by incubation in methanol or ethanol. In some embodiments, after fixation, the sample is treated to permeabilized and allow access to the interior of the structural components by enzymes and DNA tags (e.g., recording tags, probe tags or copies thereof, barcodes, or other nucleic acids).

In some embodiments, one or more washing steps are performed before and/or after fixation and/or permeabilization. Commercial fixation and permeabilization kits can be used to prepare the sample. In some embodiments, the fixing or cross-linking of the sample may be reversed.

In some embodiments, reversal of fixation or cross-linking of the sample is performed prior to isolating the macromolecules (e.g., proteins, polypeptides, or peptides) and associated recording tags from the spatial sample. In some embodiments, reversal of fixation or cross-linking of the sample is performed after isolating the macromolecules (e.g., proteins, polypeptides, or peptides) and associated recording tags from the spatial sample. For example, crosslinking may be reversed by incubating the cross-linked sample in high salt (approximately 200 mM NaCl) at 65° C. for about four hours or more.

In some embodiments, a tissue sample will be treated to remove embedding material (e.g. to remove paraffin or formalin) from the sample prior to release, capture or treatment of the macromolecules (e.g., proteins, polypeptides, or peptides) from the spatial sample. This can be achieved by contacting the sample with an appropriate solvent (e.g. xylene and ethanol washes). Treatment can occur prior to contacting the tissue sample with a solid support set forth herein or the treatment can occur while the tissue sample is on the solid support.

2. Providing a Recording Tag

The methods provided herein include providing a spatial sample comprising one or more macromolecules (e.g., proteins, polypeptides, or peptides) with a recording tag. In some embodiments, the spatial sample is provided with a plurality of recording tags. In some aspects, a plurality of macromolecules in a spatial sample is provided with recording tags. The recording tags may be associated or attached, directly or indirectly to the macromolecules or other moieties in the spatial sample. In some embodiments, the recording tags are attached to the macromolecules using any suitable means. In some embodiments, a macromolecule may be associated with one or more recording tags. In some aspects, the recording tag may be any suitable sequenceable moiety to which information from the probe tag, and optionally identifying information of one or more coding tags, can be transferred. The recording tag serves as a moiety to which information, such as information regarding a molecular probe, can be transferred or recorded.

In some other embodiments, the recording tags are not associated or attached, directly or indirectly to the macromolecules or other moieties in the spatial sample but are held in place in a matrix, scaffold, or substance applied to the spatial sample. In some embodiments, the spatial sample is exposed to a matrix (e.g., a polymer matrix), scaffold, or other substance containing recording tags. See e.g., Gao et al., BMC Biology (2017) 15:50). For example, the matrix may comprise hydrogel polymer chains. In some embodiments, the spatial sample (e.g., a biological tissue or specimen) is chemically fixed and treated with compounds that bind to macromolecules such that the biomolecules are tethered to hydrogel polymer chains. For example, a hydrogel made of closely spaced, densely cross-linked, highly charged monomers is polymerized evenly throughout the cells or tissue in the spatial sample, intercalating between and around the macromolecules and biomolecules in the spatial sample. In some cases, the embedded spatial sample can be exposed to a mechanical homogenization step involving denaturation and/or digestion of structural molecules. In some embodiments, a spatial sample comprises a specimen-hydrogel composite.

In some embodiments of the provided methods, information from a probe tag is transferred to the recording tag. The recording tag may comprise other nucleic acid components. In some embodiments, the recording tag may comprise a unique molecular identifier, a compartment tag, a partition barcode, sample barcode, a fraction barcode, information transferred from a probe tag, a spacer sequence, a universal priming site, or any combination thereof.

In embodiments of the methods including a macromolecule analysis assay, at least one recording tag is associated or co-localized directly or indirectly with the macromolecule (e.g., polypeptide). In a particular embodiment, a single recording tag is attached to a polypeptide, preferably via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

In some embodiments, the density or number of macromolecules provided with a recording tag is controlled or titrated. In other embodiments, the matrix or substance containing recording tags applied to the spatial sample is titrated for a desired density of recording tags. For example, it may be desirable to space the recording tags in or on the spatial sample appropriately to accommodate methods to be used to assess the spatial location of the macromolecules. In some cases, the amount or density of recording tags associated with macromolecules in the spatial sample is titrated on the surface of the sample or within the volume of the sample.

In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a macromolecule in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

A recording tag may comprise DNA, RNA, or polynucleotide analogs including PNA, γPNA, GNA, BNA, XNA, TNA, other polynucleotide analogs, or a combination thereof. A recording tag may be single stranded, or partially or completely double stranded. A recording tag may have a blunt end or overhanging end. In some embodiments, the recording tag may comprise a peptide or sequence of amino acids. In some cases, the recording tag is a moiety that allows a sequence of amino acids (e.g., a peptide barcode) to be attached or added.

In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of macromolecules within a sample are labeled with recording tags. In a particular embodiment, a subset of macromolecules from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the macromolecules in the spatial sample in situ. In some embodiments, the recording tags are attached to the macromolecules prior to providing the sample on a solid support. In some embodiments, the recording tags are attached to the macromolecules after providing the sample on the solid support. In some other embodiments, the recording tags are not associated or attached, directly or indirectly to the macromolecules or other moieties in the spatial sample but are provided in a matrix, scaffold, or substance applied to the spatial sample.

In some embodiments, the recording tag can also include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a solid support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual macromolecules. In some embodiments, within a library of macromolecules, each macromolecule is associated with a single recording tag, with each recording tag comprising a unique UMI. In other embodiments, multiple copies of a recording tag are associated with a single macromolecule, with each copy of the recording tag comprising the same UMI. In some embodiments, a UMI has a different base sequence than the spacer or encoder sequences within the binding agents' coding tags to facilitate distinguishing these components during sequence analysis. In some embodiments, the UMI may provide function as a location identifier and also provide information in the macromolecule analysis assay. For example, the UMI may be used to identify molecules that are identical by descent, and therefore originated from the same initial molecule. In some aspects, this information can be used to correct for variations in amplification, and to detect and correct sequencing errors.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:1) or an Illumina P7 primer (5′-CAAGCAGAAGACGGCATACGAGAT-3′-SEQ ID NO:2).

The recording tags may comprise a reactive moiety for a cognate reactive moiety present on the target macromolecule, e.g., the target protein (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule. A suitable linker can be attached to various positions of the recording tag, such as the 3′ end, at an internal position, or within the linker attached to the 5′ end of the recording tag.

B. Molecular Probe

The methods provided herein include binding of one or more molecular probes to the spatial sample. In some embodiments, the molecular probe comprises a detectable label and a probe tag. After providing a spatial sample comprising one or more macromolecules with one or more recording tags, the method includes applying and binding one or more molecular probes to the spatial sample. The spatial sample may include any sample of interest, such as described and optionally treated as described above. In some embodiments, prior to binding of the spatial sample with one or more molecular probes, the spatial sample is treated with a blocking agent.

In some embodiments, two or more molecular probes are applied to the spatial sample. In some cases where a plurality of molecular probes are used, molecular probes of the same identity are associated with the same probe tag. In some embodiments, each molecular probe in the plurality of molecular probes is associated with a unique detectable label. In some embodiments, two or more probes are associated with the same detectable label. The one or more molecular probes may be applied sequentially or a plurality of molecular probes may be applied at the same time. In some cases, molecular probes of different identities are associated with the same probe tag. In some cases, molecular probes of different identities are associated with the same detectable label. In some aspects, molecular probes of different identities may be associated with the same detectable label due to a limited number of detectable labels available. In some cases, the method may include decoding combinatorial information from transferring two or more probe tags serially to the recording tag. In some particular embodiments, the sample is provided with a plurality of molecule probes, wherein some molecular probes associated with a detectable label and some are not associated with a detectable label (e.g. a “dummy molecular probe”).

The molecular probe may be comprised of any composition suitable for binding the spatial sample. In some examples, the molecular probe comprises a nucleic acid, a peptide, a polypeptide, a protein, carbohydrate, or a small molecule that binds to, associates, unites with, recognizes, or combines with the spatial sample. The molecular probe may form a covalent association or non-covalent association with the spatial sample or a component of the spatial sample. In some aspects, the molecular probe may form a reversible association with the spatial sample or a component of the spatial sample. A molecular probe may be a chimeric molecule, composed of two or more types of molecules, such as a nucleic acid molecule-peptide chimeric molecular probe or a carbohydrate-peptide chimeric molecular probe. A molecular probe may be a naturally occurring, synthetically produced, or recombinantly expressed molecule. A molecular probe may bind to a linear molecule or a molecule having a three-dimensional structure (also referred to as conformation).

In some examples, the molecular probe comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

In some embodiments, the molecular probe comprises a microprotein (cysteine knot protein, knottin), a DARPin; a Tetranectin; an Affibody; an Affimer, a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)2, a peptide mimetic molecule, or a synthetic molecule (See e.g., Nelson, MAbs (2010) 2(1): 77-78, Goltsev et al., Cell. 2018 Aug. 9; 174(4):968-981, or as described in US Patent Nos. or Patent Publication Nos. U.S. Pat. Nos. 5,475,096, 5,831,012, 6,818,418, 7,166,697, 7,250,297, 7,417,130, 7,838,629, US 2004/0209243, and/or US 2010/0239633).

In some embodiments, the molecular probe is capable of chemically binding, covalently binding, and/or reversible binding to the spatial sample. In some embodiments, the molecular probe binds to a moiety that is bound to, associated with or complexed with the macromolecule in the spatial sample. In some examples, the molecular probe binds to a macromolecule (e.g., target macromolecule), a moiety in proximity to the macromolecule, or a moiety associated or bound to the macromolecule in the spatial sample. In some embodiments, the molecular probe binds a moiety in proximity to the macromolecule such that transfer of information from a probe tag can be transferred to a recording tag allow association with the molecular probe. For example, the distance between the macromolecule and the moiety in proximity to the macromolecule is about 10 nm to 100 nm; about 10 nm to 500 nm, about 10 nm to 1,000 nm, about 10 nm to 5,000 nm, about 100 nm to 300 nm; about 100 nm to 600 nm; about 100 nm to 1,000 nm; about 100 nm to 5,000 nm; about 300 nm to 600 nm, about 300 nm to 1,000 nm; or 300 nm to 5,000 nm. In some cases, transfer of information from the probe tag to the recording tag can occur if the recording tag is in proximity to the probe tag, regardless where the molecular probe is bound to the macromolecule. In some embodiments, the molecular probe is attached to the probe tag via a linker which may be of various lengths. In some cases, the length of the linker between the molecular probe and the probe tag may increase the distance between a moiety in proximity to the molecular probe and the molecular probe which allows association to the molecular probe. In some embodiments, the proximity of the moiety to the macromolecule may depend on the length of any linkers used in the molecular probe to attach the probe tag.

In some examples, the targeting moiety is configured to bind to a macromolecule, including but not limited to a nucleic acid, a carbohydrate, a lipid, a polypeptide, a post-translational modification of a polypeptide, or any combinations thereof. In some embodiments, the targeting moiety is a protein-specific targeting moiety, an epitope-specific targeting moiety, or a nucleic acid-specific targeting moiety. In some cases, the molecular probe is configured to bind to a cell surface marker. In some embodiments, the targeting moiety binds to a post-translational modifications (PTMs) of a polypeptide or amino acid. Examples of PTMs include but is not limited to phosphorylation, ubiquitination, methylation, acetylation, glycosylation, oxidation, lipidation, nitrosylation, SUMOylation, ubiquitination, and others.

In some embodiment, the molecular probe comprises a targeting moiety capable of specific and/or selective binding. In some embodiment, the molecular probe comprises a targeting moiety capable of specific or partially specific binding. An example of a structure-specific binder may include a protein-specific molecule that may bind to a protein target. Examples of suitable protein-specific molecules may include antibodies and antibody fragments, nucleic acids (for example, aptamers that recognize protein targets), or protein substrates. In some embodiments, a target of the targeting moiety may include an antigen and a molecular probe may include an antibody. A suitable antibody may include monoclonal antibodies, polyclonal antibodies, multi-specific antibodies (for example, bispecific antibodies), or antibody fragments so long as they bind specifically to a target antigen. In some embodiments, the molecular probe comprises a moiety or a nucleic acid component configured to specifically bind nucleic acids, such as a specific target nucleic acid sequence.

The molecular probes provided herein may comprise any suitable detectable label, including but not limited to radioisotopes, fluorescent labels, colorimetric labels, and various enzyme-substrate labels know in the art. In some embodiments, the signal from the detectable label can be amplified by binding a secondary probe to the primary molecular probe. For example, the secondary probe may be fluorescently labeled or may be conjugated to an enzyme that can then amplify a signal.

In some embodiments, the detectable label or a secondary probe is detectable visually by microscopy or using an imager. In certain cases, the fluorophore used may be a coumarin, a cyanine, a benzofuran, a quinoline, a quinazolinone, an indole, a benzazole, a borapolyazaindacene and or a xanthene including fluorescein, rhodamine and rhodol. In multiplexing embodiments, fluorophores may be chosen so that they are distinguishable, i.e., independently detectable, from one another, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same area of the section). Specific fluorescent dyes of interest include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachloro fluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, Naptho fluorescein, Texas Red, Cy3, and Cy5, etc.

In some embodiments, the present method includes one or more cycles of binding of the molecular probe to the spatial sample and assessing, e.g., observing, the detectable label of the molecular probe. In some embodiments, one or more cycles of binding of molecular probes and assessing, e.g., observing, the detectable label may be performed using an system, such as an automated system. In some embodiments, a microfluid system for cell analysis can be used which delivers and applies the reagents for the provided methods. In some aspects, the system for performing one or more steps of the method may be multiplex. For example, a multiplexed tissue processing platform may be utilized. In some embodiments, a microfluidic flow cell may be used for the binding of the molecular probes to the spatial sample and/or the observation of the detectable labels (e.g., Cell DIVE™ from GE Research).

In some embodiments, the method may include assessing, e.g., observing, some of the detectable labels of the provided molecular probes. In some particular cases, the spatial sample is provided with one or more molecular probes that is not labeled with a detectable signal. In some cases, the method does not require the detection of all molecular probes contacted with the spatial sample. For example, the probe tag of a molecular probe can be transferred to the recording tag without observing any detectable label associated with said molecular probe.

In some embodiments, signal intensity, signal wavelength, signal location, signal frequency, or signal shift of the detectable label associated with the molecular probe is observed. In some embodiments, the observation of the detectable label may be performed prior to transfer of the information from the probe tag to the recording tag. In some cases, the observation of the detectable label may be performed after transfer of the information from the probe tag to the recording tag. In some embodiments, one or more aforementioned characteristics of the signal may be observed, measured, and recorded. In some embodiments, a detectable label may include a fluorophore and fluorescence wavelength or fluorescent intensity may be determined using a fluorescence detection system.

A signal from detectable label may be detected using a detection system. Examples include microscopes configured for light, bright field, dark field, phase contrast, fluorescence, reflection, interference, and/or confocal imaging. The detection system may include an electron spin resonance (ESR) detection system, a charge coupled device (CCD) detection system (e.g., for radioisotopes), a fluorescent detection system, an electrical detection system, a photographic film detection system, a chemiluminescent detection system, an enzyme detection system, an atomic force microscopy (AFM) detection system (for detection of microbeads), a scanning tunneling microscopy (STM) detection system (for detection of microbeads), an optical detection system, a near field detection system, or a total internal reflection (TIR) detection system.

In some embodiments, assessing, e.g., observing, the detectable label may include capturing an image of the spatial sample. In some examples, the assessing, e.g., observing, the detectable label comprises obtaining a digital image of the spatial sample or a portion thereof. In some embodiments, a microscope connected to an imaging device may be used as a detection system, in accordance with the methods disclosed herein. In some embodiments, a detectable label (such as, fluorophore) may be excited and the signal (such as, fluorescence signal) obtained may be observed and recorded in the form of a digital signal (for example, a digitalized image). The same procedure may be repeated for different detectable labels (if present, such as on multiple molecular probes) that are bound in the sample using the appropriate fluorescence filters. In some embodiments, the method includes overlaying all of the images to produce an image showing the pattern of binding of all of the molecular probes to the sample.

In some embodiments of the methods provided herein, the method includes a step of acquiring at least one image of the spatial sample. In some cases, two or more digital images of the spatial sample are obtained. For example, the two or more digital images may provide combinatorial spatial information of the plurality of molecular probes. In some embodiments, the method may also include comparing, aligning, and/or overlaying at least two of the images. The assessing, e.g., observing, may be performed on a spatial sample that is in contact with a solid support. The image may include an image of the detectable label and/or spatial information of the sample. An image can be obtained using detection devices known in the art and as described above. A spatial sample containing a biological specimen can be stained prior to imaging to provide morphological or anatomical information, including to visualize different regions or cells. In some embodiments, more than one stain can be used to image different aspects of the specimen (e.g. different regions of a tissue, different cells, specific subcellular components or the like). In other embodiments, a spatial sample containing a biological specimen can be imaged without staining. In some cases, different images can be registered to each other (including correcting for distortions or warping of image and/or sample) by making use of features in the image. For example, fiducial registration markers can be introduced for this purpose or other types of marker detectable across images can be used.

In some examples, the provided methods can be used with other methods to identify features of a spatial sample, e.g. optical images of the spatial sample and/or images of histological staining. In some examples, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic reticulum, golgi body, nuclear envelope, and so forth), a compartment of the cell (e.g., cytosol, nuclear fraction, and so forth). The stain may enhance contrast or imaging of intracellular or extracellular structures. In some embodiments, the sample may be stained with haematoxylin and eosin (H&E). By combining other types of information, a richer spatial context for interpreting the protein information may be useful.

In some embodiments, the method includes correlating locations in an image of the sample with probe tags associated with a molecular probe. Accordingly, characteristics of the spatial sample containing a biological specimen that are identifiable in the image can be correlated with the molecular probes bound to the same location of the spatial sample. Any of a variety of morphological characteristics can be used in such a correlation, including for example, cell shape, cell size, tissue shape, staining patterns, presence of particular proteins (e.g. as detected by immunohistochemical stains) or other characteristics that are routinely evaluated in pathology or research applications. Accordingly, the biological state of a tissue or its components as determined by visual observation can be correlated with the molecular probes and information of the macromolecules from the macromolecule analysis assay.

In some embodiments, the method includes inactivating the detectable label after assessment or observation of the detection of the label is performed. For example, chemical inactivation of fluorescent dyes after each image acquisition round may be performed. In some embodiments, the molecular probe is removed after detection of the detectable label is performed. In an example, the method includes cycles of binding of the molecular probe to the spatial sample, observing the detectable label, and washing to remove the molecular probe. In some embodiments, the detectable label is inactivated prior to binding a new molecular probe to the sample. In some examples, the sample is treated with an inactivation solution to inactivate the detectable label. For example, the sample may be treated with alkaline oxidation chemistry to inactivate a dye. See e.g., Gerdes et al., Proc Natl Acad Sci USA. (2013) 110(29): 11982-11987.

C. Transfer of Probe Tag Information

In the methods provided herein, the molecular probe comprises a probe tag comprising information to be transferred to the recording tag. In some embodiments, the information from a plurality of probe tags is transferred to a plurality of recording tags. In some embodiments involving transfer of information from more than one probe tag to a recording tag, the information from each probe tag is transferred sequentially to the recording tag. In some embodiments, the information from one probe tag is transferred to two or more recording tags. In some embodiments, the information from more than one probe tag is transferred to a recording tag. In some embodiments, the probe tag comprises at least one barcode. In some embodiments, the transferred information from the probe tag to the extended recording tag may also be referred to as a probe tag. In some aspects, the extended recording tag comprises a probe tag sequence. In some cases, the transferred probe tag sequence may be complementary to the probe tag sequence associated or attached to the molecular probe.

In some embodiments, the use of the molecular probes may include adjustments useful for subsampling and/or tuning the dynamic range. In some cases, the concentration of molecular probes provided to the sample can be tuned and adjusted. For example, for detection of single molecules, the concertation of the molecular probes provided can be reduced. In some embodiments, the sample is provided with a plurality of molecule probes, wherein some molecular probes are labeled with a probe tag and some are not labeled with a probe tag (e.g. a “dummy molecular probe”). In some cases, the sample is provided with a plurality of molecular probes that includes at least about 10%, 15%, 20%, 25%, 30%, 35%, 40%, 45%, 50%, 55%, 60%, 65%, 70%, 75%, 80%, 85%, 90%, 95%, 97%, or 99% molecular probes that are not labeled with a probe tag (e.g. “dummy molecular probes”). In some aspects, the sample is provided with a plurality of molecule probes, wherein two or more of the same molecular probes are associated with different probe tags.

A plurality of macromolecules of the spatial sample can be labeled with a probe tag or contain information transferred from a probe tag comprising the same barcode. In some embodiments, a plurality of recording tags in proximity to probe tags associated with molecular probes can be extended by transferring information from the probe tags. The recording tags need not be attached or associated to the moiety bound by the molecular probe as long as the recording tags are in proximity to the probe tag. In some embodiments, information of a probe tag may be transferred to a recording tag that is in proximity, wherein the probe tag is indirectly associated with the moiety bound by the molecular probe. For example, the distance between the recording tag and the moiety or macromolecule bound by the molecular probe comprising the probe tag is about 10 nm to 100 nm; about 10 nm to 500 nm, about 10 nm to 1,000 nm, about 10 nm to 5,000 nm, about 100 nm to 300 nm; about 100 nm to 600 nm; about 100 nm to 1,000 nm; about 100 nm to 5,000 nm; about 300 nm to 600 nm, about 300 nm to 1,000 nm; or 300 nm to 5,000 nm. In some examples, a plurality of macromolecules within a cell may be labeled with a probe tag or contain information transferred from a probe tag comprising the same barcode. In some examples, a plurality of macromolecules within an organelle may be labeled with a probe tag or contain information transferred from a probe tag comprising the same barcode.

In some embodiments, a probe tag is a nucleic acid tag comprising a barcode that is transferred to the recording tag associated with the macromolecules in the spatial sample. In some embodiments, probe tag information is transferred to the recording tag by generating the sequence in situ on the recoding tag associated with the macromolecule in the spatial sample. By transferring the information from the probe tag to the recording tag, in some embodiments, the recording tag comprises a probe tag. In some examples, the method includes generating in situ a sequence on the recording tag that contains a barcode sequence from the probe tag. In some embodiments, the probe tag is physically transferred to the recording tag. In some cases, the probe tag is generated or attached using chemical/enzymatic reactions, such as ligation or polymerase or primer extension, onto the recording tag.

In certain embodiments, a probe tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length.

The probe tag may be any suitable tag. In some examples, the probe tag comprises a DNA molecule, DNA with pseudo-complementary bases, an RNA molecule, a BNA molecule, an XNA molecule, a LNA molecule, a PNA molecule, or a γPNA molecule. In some embodiments, the probe tag comprises a non-nucleic acid sequenceable polymer, e.g., a polysaccharide, a polypeptide, a peptide, or a polyamide, or a combination thereof. In some embodiments, the probe tag is a nucleic acid. In some embodiments, the probe tag comprises a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length. A probe tag may comprise a barcode sequence, which is optionally flanked by one spacer on one side or flanked by a spacer on each side. A probe tag may be single stranded or double stranded. A double stranded probe tag may comprise blunt ends, overhanging ends, or both. A probe tag may refer to the probe tag that is directly attached to a molecular probe, to a complementary sequence to the probe tag that is directly attached to a molecular probe, or to probe tag information present in an extended recording tag.

In certain embodiments, a probe tag comprises a barcode. A barcode is a nucleic acid molecule of about 3 to about 30 bases, about 3 to about 25 bases, about 3 to about 20 bases, about 3 to about 10 bases, about 3 to about 10 bases, about 3 to about 8 bases in length. In some embodiments, a barcode is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In one embodiment, a barcode allows for multiplex sequencing of a plurality of samples or libraries. Barcodes can be used to de-convolute multiplexed sequence data and identify sequence reads from an individual sample or library. In some embodiments, the probe tag comprises more than one barcode. For example, the probe tag can be comprised of a string of 2 or more tags, each being a barcode. In some aspects, a concatenated string of barcodes can allow increased diversity of barcodes for labeling or identifying. For example, if 10 different tags (e.g., barcodes) are used and concatenated in a random way into a string of 3 tags as a barcode, then the concatenated barcode would have 10³=1000 possible sequences by using 10 tags arranged in a combinatorial manner. In some embodiments, a string of probe tags used in a combinatorial manner may be used to provide information regarding one or more molecular probes. For example, the recording tag may contain information in a series from one, two, three, four, five, six, seven, eight, nine, ten, or more probe tags.

In some embodiments, the probe tag comprises a peptide or amino acid barcode, that comprises a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode that can be distinguished from other peptide barcodes can have different physical characteristics (amino acid sequence, sequence length, charge, size, molecular weight, hydrophobicity, reverse phase separation, affinity or other separable property). See e.g., International Patent Publication Nos. WO2016145416 and WO2018/078167. The probe tag may comprise a barcode that is associated with one or more molecular probes. The molecular probes may be associated with or attached to the peptide barcode using any suitable means, including but not limited to any enzymatic or chemical attachment means. The information of the peptide barcode of the probe tag can be transferred to the recording tag using any suitable means, including but not limited to any enzymatic or chemical attachment means. See e.g., Miyamoto et al., PLoS One. (2019) 14(4):e0215993; Wroblewska et al., Cell. (2018)175(4):1141-1155.e16. In some embodiments, linkers made of amino acid sequences that are typically flexible permitting the attachment of two different polypeptides can be used. For example, a linear linking peptide consists of between two and 25 amino acids, between two and 15 amino acids, or longer linkers can be used.

In some embodiments, the probe tag comprises a spacer. In some embodiments, the spacer on the probe tag is configured to hybridize to a sequence comprise by the recording tag. In some embodiments, the probe tag comprises a universal priming site. In some embodiments, the probe tag further comprises other nucleic acid components. In some embodiments, the probe tag further comprises a universal priming site.

Information from the probe tag may be transferred to the recording tag in any suitable manner. For example, information from the probe tag may be transferred to the recording tag by extension or ligation. In some cases, ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof can be used to transfer information from the probe tag to the recording tag to generate an extended recording tag. In some embodiments, transferring information from the probe tag to the recording tag comprises contacting the spatial sample with a polymerase and a nucleotide mix, thereby adding one or more nucleotides to the recording tag. In some cases, the probe tag in the molecular probe serves as a template for extension. In certain embodiments, information of a probe tag is transferred to a recording tag via primer extension (Chan et al., Curr Opin Chem Biol. (2015) 26: 55-61). A spacer sequence on the 3′-terminus of a recording tag anneals with complementary spacer sequence on the 3′ terminus of a probe tag and a polymerase (e.g., strand-displacing polymerase) extends the recording tag sequence, using the annealed probe tag as a template.

In some embodiments, information from the probe tag is capable of being transferred to any recording tag in the proximity of the probe tag. The distance between the position in the spatial sample bound by the molecular probe and a recording tag which allows the probe tag information to be transferred to the recording tag may depend on the distance a probe tag and recording tag may reach. For example, a molecular probe may be a nucleic acid that binds a target nucleic acid and the target nucleic acid is bound to a polymerase. In this example, the polymerase is attached to a recording tag and the recording tag is in the vicinity of the probe tag attached to the target nucleic acid. In another example, a recording tag contained in a matrix applied to the spatial sample may be in proximity to a probe tag attached to a molecular probe that is bound to a polypeptide in the spatial sample.

The transferring of information from the probe tag to a recording tag can be directly from the probe tag in the molecular probe or indirectly via a copy of the probe tag. In some embodiments, the probe tag in the molecular probe is copied one or more times prior to transferring the information of the probe tag to a recording tag. For example, the probe tag in the molecular probe may be amplified before transferring the information of the probe tag to a recording tag. In some cases, the amplification of the probe tag is linear amplification. In some aspects, the amplification of the probe tag is performed using a RNA polymerase. In cases where copies of the probe tag comprises RNA, the transferring of the probe tag to the recording tag may be performed using reverse transcription. In one example, the molecular probe may bind to a cell surface marker and polypeptides inside the cells are associated with recording tags. In this case, copies of the probe tag attached to the molecular probe bound to the outside of the cell is made, and the copies of the probe tag may then diffuse into the cells and transfer of information from the copies of the probe tag to the recording tags attached to macromolecules inside the cells may occur.

Following transfer of information from the probe tag to the recording tag, macromolecules associated with recording tags that contain information from one or more probe tags is used in a macromolecule analysis assay. In some aspects, the macromolecule analysis assay is a polypeptide analysis assay. In some embodiments, the probe tag comprises a barcode which can be used to provide or derive information regarding the spatial location of the protein within the spatial sample. The barcode may allow for multiplex sequencing of a plurality of samples or libraries from tissue section(s).

Optionally, the spatial sample or any portion thereof can be removed from a solid support after transfer of information from the one or more probe tags to the recording tags and after one or more images of the detectable label has been obtained. Thus, a method of the present disclosure can include a step of washing a solid support to remove macromolecules, cells, tissue or other materials from the spatial sample. Removal of the spatial sample or any portion thereof can be performed using any suitable technique and will be dependent on the sample. In some cases, the solid support can be washed with water containing various additives, such as surfactants, detergents, enzymes (e.g., proteases and collagenases), cleavage reagents, or the like, to facilitate removal of the specimen. In some embodiments, the solid support is treated with a solution comprising a proteinase enzyme. In some embodiments, macromolecules are released during or after the specimen is removed from the solid support. In some embodiments, the method includes releasing and/or collecting extended recording tags from the spatial sample. In some embodiments, the extended recording tags released and/or collected contain at least one probe tag.

### IV. MACROMOLECULE ANALYSIS ASSAY

In the methods provided, the macromolecules (e.g., polypeptide) associated with a recording tag comprising information transferred from one or more probe tags and spatial tags are optionally used in a macromolecule analysis assay. In some embodiments, the macromolecules with associated and/or attached recording tags (containing information transferred from one or more probe tags and spatial tags) are subjected to a polypeptide analysis assay. In some examples, the macromolecule analysis assay is performed on macromolecules released from the spatial sample. In a preferred embodiment, macromolecules with attached extended recording tags are released from the sample prior to performing the macromolecule analysis assay. The macromolecule analysis assay is performed to identify or determine at least a portion of the sequence, or assess the macromolecule. In some aspects, the provided methods provide spatial information with the information obtained from performing a macromolecule analysis assay.

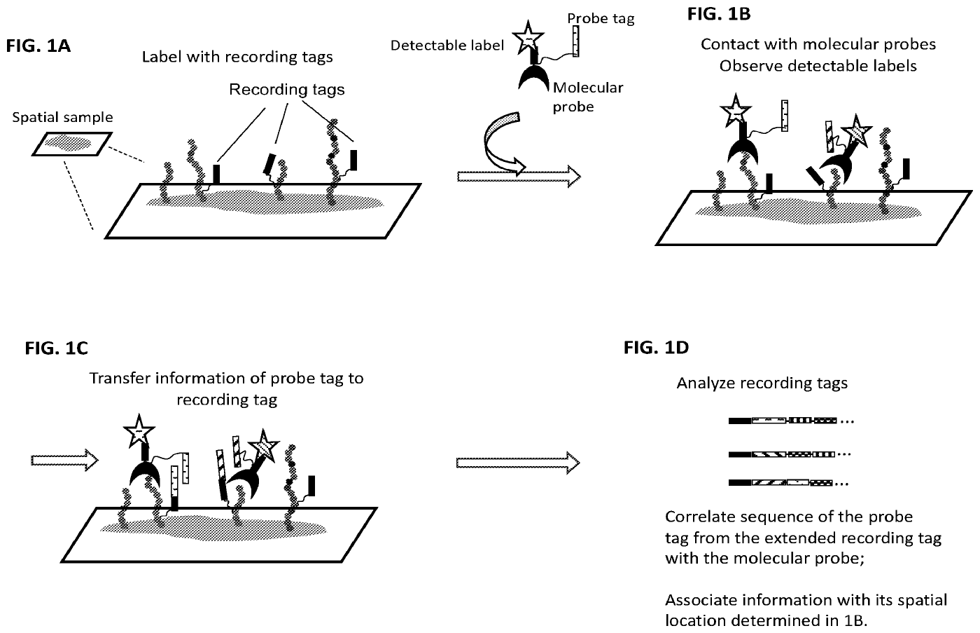

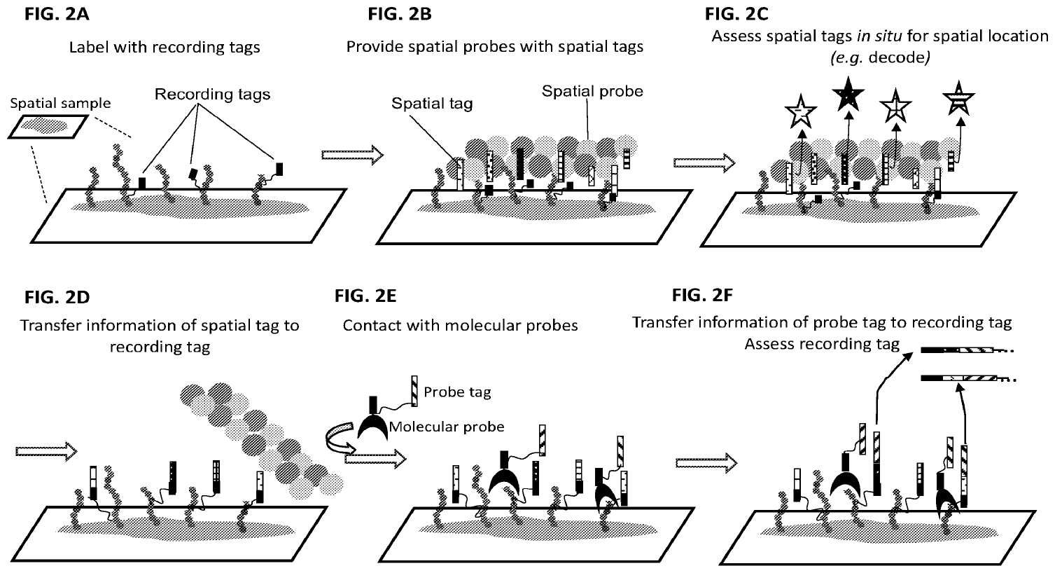

In an exemplary preparation method, a sample is prepared for spatial analysis by fixing and embedding a tissue sample in paraffin analysis (e.g., FFPE (formalin-fixed, paraffin-embedded sample), followed by sectioning the embedded tissue sample. The planar sections may then be attached to or provided on a slide. During the sample preparation, macromolecules in the spatial sample are provided with recording tags. The spatial sample is provided with a plurality of molecular probes each comprising a probe tag and optionally a detectable label. The molecular probes bind to the spatial sample and information from the probe tags associated with the molecular probes are transferred to the recording tags associated with macromolecules in the sample. Assessing of spatial location of the macromolecules in the sample may include 1) assessing the detectable label of the molecular probe, including observing the detectable label one or more times to obtain spatial information of the molecular probe, as shown in FIG. 1A-D; or 2) assessing a spatial tag provided to the spatial sample in situ to obtain the spatial location of the spatial tag in the spatial sample, as shown in FIG. 2A-2F. The macromolecules with the associated recording tags (containing information transferred from one or more probe tags) are subjected to a macromolecule analysis assay.

In some embodiments, the macromolecule analysis assay is a next generation protein assay (NGPA) using multiple binding agents and enzymatically-mediated sequential information transfer. In some cases, the analysis assay is performed on immobilized protein molecules simultaneously bound by two or more cognate binding agents (e.g., antibodies). After multiple cognate antibody binding events, a combined primer extension and DNA nicking step is used to transfer information from the coding tags of bound antibodies to the recording tag. In some cases, polyclonal antibodies (or mixed population of monoclonal antibody) to multivalent epitopes on a protein can be used for the assay. See e.g., International Patent Publication Nos. WO 2017/192633. In some particular embodiments, the polypeptide analysis assay can be performed to assay a peptide barcode (e.g., from the probe tag and/or spatial tag).

In some embodiments, the macromolecule is a polypeptide and a polypeptide analysis assay is performed. The macromolecule analysis assay may include contacting the macromolecule with a binding agent capable of binding to the macromolecule, wherein the binding agent comprises a coding tag with identifying information regarding the binding agent; and transferring the information of the coding tag to the recording tag to generate the extended recording tag (containing probe tag information). The contacting of the macromolecule with a binding agent capable of binding to the macromolecule, wherein the binding agent comprises a coding tag with identifying information regarding the binding agent; and transferring the information of the coding tag to the recording tag to extend the recording tag maybe be repeated one or more times. In some embodiments, transferring information from the probe tag in the molecular probe to the recording tag may be performed prior to or after assessing, e.g., observing, the detectable label to obtain spatial information of the molecular probe. In some cases, the polypeptide analysis assay is performed on polypeptides in situ without releasing the polypeptides from the spatial sample. In some cases, the polypeptide analysis assay is performed on polypeptides released from the spatial sample. In some cases, the polypeptide analysis assay is performed on polypeptides in situ without releasing the polypeptides from the spatial sample. In some embodiments, the sequence (or a portion of the sequence thereof) and/or the identity of a protein is determined using a polypeptide analysis assay. In some embodiments, the proteins from the spatial sample may be processed or further treated, such as with one or more enzymes and/or reagents.

In some examples, the polypeptide analysis assay includes assessing at least a partial sequence or identity of the polypeptide using suitable techniques or procedures. For example, at least a partial sequence of the polypeptide can be assessed by N-terminal amino acid analysis or C-terminal amino acid analysis. In some embodiments, at least a partial sequence of the polypeptide can be assessed using a ProteoCode assay. In some examples, at least a partial sequence of the polypeptide can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Publication Nos. WO 2017/192633, and WO/2019/089836, and WO 2019/089851.

In embodiments relating to methods of analyzing peptides or polypeptides, the method generally includes contacting and binding of a binding agent to terminal amino acid (e.g., NTAA) of a peptide and transferring the binding agent's coding tag information to the recording tag associated with the peptide, thereby generating a first order extended recording tag. The terminal amino acid bound by the binding agent may be a chemically labeled or modified terminal amino acid. In some embodiments, the terminal amino acid (e.g., NTAA) is eliminated. The terminal amino acid eliminated may be a chemically labeled or modified terminal amino acid. Removal of the NTAA by contacting with an enzyme or chemical reagents converts the penultimate amino acid of the peptide to a terminal amino acid. The polypeptide analysis may include one or more cycles of binding with additional binding agents to the terminal amino acid, transferring information from the additional binding agents to the extended nucleic acid thereby generating a higher order extended recording tag containing information from two or more coding tags, and eliminating the terminal amino acid in a cyclic manner. Additional binding, transfer, labeling, and removal, can occur as described above up to n amino acids to generate an nt^{h} order extended nucleic acid, which collectively represent the peptide. In some embodiments, steps including the NTAA in the described exemplary approach can be performed instead with a C terminal amino acid (CTAA).

In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent.

In some embodiments, the method optionally comprises collecting the protein with the associated extended recording tag (comprising information form the probe tag and/or spatial tag) prior to performing the protein (e.g., polypeptide) analysis assay. In some embodiments, the methods optionally comprise releasing the proteins from the spatial sample. The polypeptide analysis assay may utilize the extended recording tag by further transferring information to it.

In some embodiments, the method comprises fragmenting the proteins obtained from the spatial sample. In some embodiments, the fragmenting is performed prior to the polypeptide analysis assay. In some examples, the proteins are from a proteolytic digest, or were treated with a protease. In some cases, the protease is trypsin, LysN, or LysC. In some embodiments, the proteins remain intact. In some embodiments, the protein analysis assay is performed on an intact spatial sample. In some embodiments, the protein analysis assay comprises binding agents for target proteins (or portions thereof).

In some embodiments, the macromolecules (e.g., polypeptides) released from the spatial sample are joined to a surface of a solid support before performing a polypeptide analysis assay. A solid support can be any support surface including, but not limited to, a bead, a microbead, an array, a glass surface, a silicon surface, a plastic surface, a filter, a membrane, a PTFE membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, silica, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a solid support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof.

As used herein, the term “solid support”, “solid surface”, or “solid substrate”, or “sequencing substrate”, or “substrate” refers to any solid material, including porous and non-porous materials, to which a macromolecule, e.g., a polypeptide, can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, or a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

Various reactions may be used to attach the polypeptides to a solid support. The polypeptides may be attached directly or indirectly to the solid support. In some cases, the polypeptide is attached to the solid support via a nucleic acid. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, a moiety for a Staudinger reaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like.

In some embodiments, a plurality of proteins is attached to a solid support prior to the polypeptide analysis assay. In certain embodiments where multiple proteins are immobilized on the same solid support, the proteins can be spaced appropriately to accommodate methods of analysis to be used to assess the proteins. For example, it may be advantageous to space the proteins that optimally to allow a nucleic acid-based method for assessing and sequencing the proteins to be performed. In some embodiments, the method for assessing and sequencing the proteins involve a binding agent which binds to the protein and the binding agent comprises a coding tag with information that is transferred to a nucleic acid attached to the proteins (e.g., recording tag). In some cases, information transfer from a coding tag of a binding agent bound to one protein may reach a neighboring protein.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of analytes (e.g., proteins, polypeptide, or peptides) can be titrated on the surface or within the volume of a solid substrate by spiking a competitor or “dummy” reactive molecule when immobilizing the proteins, polypeptides or peptides to the solid substrate.

To control protein spacing on the solid support, the density of functional coupling groups for attaching the protein (e.g., TCO or carboxyl groups (COOH)) may be titrated on the substrate surface. In some embodiments, multiple proteins are spaced apart on the surface or within the volume (e.g., porous supports) of a solid support such that adjacent proteins are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple a proteins are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple a proteins are spaced apart on the surface of a solid support with an average distance of at least 50 nm. In some embodiments, proteins are spaced apart on the surface or within the volume of a solid support such that, empirically, the relative frequency of inter- to intra-molecular events (e.g. transfer of information) is <1:10; <1:100; <1:1,000; or <1:10,000.

In some embodiments, the plurality of proteins is coupled on the solid support spaced apart at an average distance between two adjacent proteins which ranges from about 50 to 100 nm, from about 50 to 250 nm, from about 50 to 500 nm, from about 50 to 750 nm, from about 50 to 1,000 nm, from about 50 to 1,500 nm, from about 50 to 2,000 nm, from about 100 to 250 nm, from about 100 to 500 nm, from about 200 to 500 nm, from about 300 to 500 nm, from about 100 to 1000 nm, from about 500 to 600 nm, from about 500 to 700 nm, from about 500 to 800 nm, from about 500 to 900 nm, from about 500 to 1000 nm, from about 500 to 2,000 nm, from about 500 to 5,000 nm, from about 1000 to 5,000 nm, or from about 3,000 to 5,000 nm.

In some embodiments, appropriate spacing of the polypeptides on the solid support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH2 and NH2-PEGₙ-mTet is added to the activated beads (wherein n is any number, such as 1-100). The ratio between the mPEG₃-NH₂ (not available for coupling) and NH2-PEG24-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH2 is about or greater than 1:1000, about or greater than 1:10,000, about or greater than 1:100,000, or about or greater than 1:1,000,000. In some further embodiments, the recording tag attaches to the NH2-PEGₙ-mTet. In some embodiments, the spacing of the polypeptides on the solid support is achieved by controlling the concentration and/or number of available COOH or other functional groups on the solid support.

A. Cyclic Transfer of Coding Tag Information to Recording Tags

In some embodiments, the polypeptide analysis assay includes performing an assay which utilizes the extended recording tag (comprising information transferred from the probe tag and/or spatial tag) associated with the macromolecule, e.g., the polypeptide. The recording tag associated with the polypeptide is used in the polypeptide analysis assay which includes transferring identifying information from one or more coding tags to the recording tag, thereby further extending the extended recording tag. In some embodiments, the recording tag comprises a spacer polymer. In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from its coding tag to the recording tag during the first binding cycle (e.g., via annealing of complementary spacer sequences for primer extension or sticky end ligation). In one embodiment, the spacer sequence is about 1-20 bases in length, about 2-12 bases in length, or 5-10 bases in length. The length of the spacer may depend on factors such as the temperature and reaction conditions of the primer extension reaction for transferring coding tag information to the recording tag.

In some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents.

In some aspects, the spacer sequence in the recording tag is designed to have minimal complementarity to other regions in the recording tag; likewise, the spacer sequence in the coding tag should have minimal complementarity to other regions in the coding tag. In other words, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In some embodiments, a recording tag comprises from 5′ to 3′ direction: a universal forward (or 5′) priming sequence, information transferred from the probe tag, and a spacer sequence. In some embodiments, a recording tag comprises from 5′ to 3′ direction: a universal forward (or 5′) priming sequence, information transferred from the probe tag, optionally other barcodes (e.g., sample barcode, partition barcode, compartment barcode, or any combination thereof), and a spacer sequence. In some other embodiments, a recording tag comprises from 5′ to 3′ direction: a universal forward (or 5′) priming sequence, information transferred from the probe tag, optionally other barcodes (e.g., sample barcode, partition barcode, compartment barcode, or any combination thereof), an optional UMI, and a spacer sequence.

The coding tag associated with the binding agent is or comprises a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence or a sequence with identifying information, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended nucleic acid on the recording tag. In certain embodiments, a coding tag may further comprise a binding cycle specific spacer or barcode, a unique molecular identifier, a universal priming site, or any combination thereof.

In some embodiments, the identifying information from the coding tag comprises information regarding the identity of the one or more amino acid(s) on the peptide or polypeptide bound by the binding agent.

In some examples, the final extended recording tag (including any additional tags attached) containing information from one or more binding agents is optionally flanked by universal priming sites to facilitate downstream amplification and/or DNA sequencing. The forward universal priming site (e.g., Illumina's P5-S1 sequence) can be part of the original design of the recording tag and the reverse universal priming site (e.g., Illumina's P7-S2′ sequence) can be added as a final step in the extension of the nucleic acid. In some embodiments, the addition of forward and reverse priming sites can be done independently of a binding agent.

In the methods described herein, upon binding of a binding agent to a macromolecule, e.g., a protein or peptide, identifying information of its linked coding tag is transferred to the recording tag (e.g., recording tag) associated with the peptide, thereby generating an extended recording tag. The nucleic acid associated with the protein or peptide for analysis can comprise the recording tag and information from one or more probe tags. In some embodiments, the recording tag further comprises barcodes and/or other nucleic acid components. In particular embodiments, the identifying information from the coding tag of the binding agent is transferred to the recording tag or added to any existing barcodes (or other nucleic acid components) attached thereto. The transfer of the identifying information may be performed using extension or ligation. In some embodiments, a spacer is added to the end of the recording tag, and the spacer comprises a sequence that is capable of hybridizing with a sequence on the coding tag to facilitate transfer of the identifying information.

Coding tag information associated with a specific binding agent may be transferred to a recording tag using a variety of methods. In certain embodiments, information of a coding tag is transferred to a recording tag via primer extension (See e.g., Chan et al. (2015) Curr Opin Chem Biol 26: 55-61). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell et al., 2015, Elife 4: e08646). The presence of a single-stranded binding protein can facilitate the strand displacement reaction.

The extended nucleic acid (e.g., recording tag) is any nucleic acid molecule or sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) that comprises identifying information for a macromolecule, e.g., a polypeptide, to which it is associated and/or information from a molecular probe. In certain embodiments, after a binding agent binds a polypeptide, information from a coding tag linked to a binding agent can be transferred to the nucleic acid associated with the polypeptide while the binding agent is bound to the polypeptide.

An extended nucleic acid associated with the macromolecule, e.g., the peptide, with identifying information from the coding tag may comprise information from a binding agent's coding tag representing each binding cycle performed. However, in some cases, an extended nucleic acid may also experience a “missed” binding cycle, e.g., if a binding agent fails to bind to the polypeptide, because the coding tag was missing, damaged, or defective, because the primer extension reaction failed. Even if a binding event occurs, transfer of information from the coding tag may be incomplete or less than 100% accurate, e.g., because a coding tag was damaged or defective, because errors were introduced in the primer extension reaction). Thus, an extended nucleic acid may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the coding tag information present in the extended nucleic acid may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag associated with the immobilized peptide may comprise information from multiple coding tags representing multiple, successive binding events. In these embodiments, a single, concatenated extended recording tag associated with the immobilized peptide can be representative of a single polypeptide. As referred to herein, transfer of coding tag information to the recording tag associated with the immobilized peptide also includes transfer to an extended recording tag as would occur in methods involving multiple, successive binding events.

In certain embodiments, the binding event information is transferred from a coding tag to the recording tag associated with the immobilized peptide in a cyclic fashion. Cross-reactive binding events can be informatically filtered out after sequencing by requiring that at least two different coding tags, identifying two or more independent binding events, map to the same class of binding agents (cognate to a particular protein). The coding tag may contain an optional UMI sequence in addition to one or more spacer sequences. Universal priming sequences may also be included in extended nucleic acids on the recording tag associated with the immobilized peptide for amplification and NGS sequencing.

Any binding agent described comprises a coding tag containing identifying information regarding the binding agent. A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, γPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

Coding tag information associated with a specific binding agent may be transferred using a variety of methods. In certain embodiments, information of a coding tag is transferred to a recording tag associated with the immobilized peptide via primer extension (Chan, McGregor et al. 2015). A spacer sequence on the 3′-terminus of a recording tag anneals with complementary spacer sequence on the 3′ terminus of a coding tag and a polymerase (e.g., strand-displacing polymerase) extends the nucleic acid sequence on the recording tag, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended nucleic acid. The 3′ terminal spacer, on the coding tag, remaining single stranded, preferably binds to the terminal 3′ spacer on the recording tag (or any barcodes or other nucleic acid components associated). In other embodiments, a nascent recording tag associated with the immobilized peptide can be coated with a single stranded binding protein to prevent annealing of the coding tag to internal sites.

In any of the preceding embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In some embodiments, a DNA polymerase that is used for primer extension possesses strand-displacement activity and has limited or is devoid of 3′-5 exonuclease activity. Several of many examples of such polymerases include Klenow exo-(Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, Annu. Rev. Biochem. (1997) 66:61-92); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recoding tag primes extension self-extension may be minimized by inclusion of single stranded binding proteins (T4 gene 32, E. coli SSB, etc.), DMSO (1-10%), formamide (1-10%), BSA(10-100 ug/ml), TMAC1 (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo-(Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag associated with the immobilized peptide (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In another embodiment, polymerase extension buffers are comprised of 40-120 mM buffering agent such as Tris-Acetate, Tris-HCl, HEPES, etc. at a pH of 6-9.

In some embodiments, to minimize non-specific interaction of the coding tag labeled binding agents in solution with the nucleic acids of immobilized proteins, competitor (also referred to as blocking) oligonucleotides complementary to nucleic acids containing spacer sequences (e.g., on the recording tag) can be added to binding reactions to minimize non-specific interactions. In some embodiments, the blocking oligonucleotides contain a sequence that is complementary to the coding tag or a portion thereof attached to the binding agent. In some embodiments, blocking oligonucleotides are relatively short. Excess competitor oligonucleotides are washed from the binding reaction prior to primer extension, which effectively dissociates the annealed competitor oligonucleotides from the nucleic acids on the recording tag, especially when exposed to slightly elevated temperatures (e.g., 30-50° C.). Blocking oligonucleotides may comprise a terminator nucleotide at its 3′ end to prevent primer extension.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag (or extensions thereof).

Self-priming/mis-priming events initiated by self-annealing of the terminal spacer sequence of the extended recording tag with internal regions of the extended recording tag may be minimized by including pseudo-complementary bases in the recording/extended recording tag (Lahoud, Timoshchuk et al. 2008), (Hoshika, Chen et al. 2010). Pseudo-complementary bases show significantly reduced hybridization affinities for the formation of duplexes with each other due the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper, Arar et al. 2006).

Coding tag information associated with a specific binding agent may be transferred to a nucleic acid on the recording tag associated with the immobilized peptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase, Electroligase® (See e.g., U.S. Patent Publication No. US20140378315). Alternatively, a ligation may be a chemical ligation reaction. As illustrated in International Patent Publication No. WO 2017/192633, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Peng et al., European J Org Chem (2010) (22): 4194-4197; El-Sagheeret al., Proc Natl Acad Sci USA (2011) 108(28): 11338-11343; El-Sagheer et al., Org Biomol Chem (2011) 9(1): 232-235; Sharma et al., Anal Chem (2012) 84(14): 6104-6109; Roloff et al., Bioorg Med Chem (2013) 21(12): 3458-3464; Litovchick et al., Artif DNA PNA XNA (2014) 5(1): e27896; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivitizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

A coding tag comprises an encoder sequence that provides identifying information regarding the associated binding agent. An encoder sequence is about 3 bases to about 30 bases, about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the encoder sequence determines the number of unique encoder sequences that can be generated. Shorter encoding sequences generate a smaller number of unique encoding sequences, which may be useful when using a small number of binding agents. In a specific embodiment, a set of >50 unique encoder sequences are used for a binding agent library.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. A spacer sequence is about 1 base to about 20 bases, about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, 11 bases, 12 bases, 13 bases, 14 bases, 15 bases or 20 bases in length. In some embodiments, a spacer within a coding tag is shorter than the encoder sequence, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the encoder sequence. In other embodiments, a spacer within a coding tag is the same length as the encoder sequence. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag may optionally contain pseudo complementary bases to a 3′ spacer on the recording tag to increase T. (Lehoud et al., 2008, Nucleic Acids Res. 36:3409-3419). In other embodiments, the coding tags within a library of binding agents do not have a binding cycle specific spacer sequence.

In one example, two or more binding agents that each bind to different targets have associated coding tags share the same spacers. In some cases, coding tags associated with two or more binding agents share coding tags with the same sequence or a portion thereof.

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. In some embodiments, a spacer sequence comprises a sufficient number of bases to anneal to a complementary spacer sequence in a recording tag or extended recording tag to initiate a primer extension reaction or sticky end ligation reaction.

In some embodiments, coding tags associated with binding agents used to bind in an alternating cycles comprises different binding cycle specific spacer sequences. For example, a coding tag for binding agents used in the first binding cycle comprise a “cycle 1” specific spacer sequence, a coding tag for binding agents used in the second binding cycle comprise a “cycle 2” specific spacer sequence, a coding tag for binding agents used in the third binding cycle also comprises the “cycle 1” specific spacer sequence, a coding tag for binding agents used in the fourth binding cycle comprises the “cycle 2” specific spacer sequence. In this manner, cycle specific spacers are not needed for every cycle.

A cycle specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle dependent spacer sequences. More specifically, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording tag, the cycle 2 specific spacer sequence is positioned at the 3′ terminus of the extended recording tag at the end of binding cycle 1. Coding tags of binding agents from the second binding cycle are capable of annealing to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent may fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step following binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

In one embodiment, binding cycle-specific encoder sequences are used in coding tags. Binding cycle-specific encoder sequences may be accomplished either via the use of completely unique analyte (e.g., NTAA)-binding cycle encoder barcodes or through a combinatoric use of an analyte (e.g., NTAA) encoder sequence joined to a cycle-specific barcode. The advantage of using a combinatoric approach is that fewer total barcodes need to be designed. For a set of 20 analyte binding agents used across 10 cycles, only 20 analyte encoder sequence barcodes and 10 binding cycle specific barcodes need to be designed. In contrast, if the binding cycle is embedded directly in the binding agent encoder sequence, then a total of 200 independent encoder barcodes may need to be designed. An advantage of embedding binding cycle information directly in the encoder sequence is that the total length of the coding tag can be minimized when employing error-correcting barcodes. The use of error-tolerant barcodes allows highly accurate barcode identification using sequencing platforms and approaches that are more error-prone, but have other advantages such as rapid speed of analysis, lower cost, and/or more portable instrumentation.

In some embodiments, a coding tag comprises a cleavable or nickable DNA strand within the second (3′) spacer sequence proximal to the binding agent. For example, the 3′ spacer may have one or more uracil bases that can be nicked by uracil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments is particularly useful in assays analysing proteins in their native conformation, as it allows the non-denaturing removal of the binding agent from the (extended) recording tag after primer extension has occurred and leaves a single stranded DNA spacer sequence on the extended recording tag available for subsequent binding cycles.

The coding tags may also be designed to contain palindromic sequences. Inclusion of a palindromic sequence into a coding tag allows a nascent, growing, extended recording tag to fold upon itself as coding tag information is transferred. The extended recording tag is folded into a more compact structure, effectively decreasing undesired inter-molecular binding and primer extension events.

In some embodiments, a coding tag comprises analyte-specific spacer that is capable of priming extension only on recording tags previously extended with binding agents recognizing the same analyte. An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles to identify the analyte. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

In certain embodiments, a coding tag may further comprise a unique molecular identifier for the binding agent to which the coding tag is linked. A UMI for the binding agent may be useful in embodiments utilizing extended coding tags or di-tag molecules for sequencing readouts, which in combination with the encoder sequence provides information regarding the identity of the binding agent and number of unique binding events for a polypeptide.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

A coding tag is joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin).

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In certain embodiments, an ensemble of nucleic acids on the recording tag may be employed per polypeptide to improve the overall robustness and efficiency of coding tag information transfer. The use of an ensemble of nucleic acids associated with a given polypeptide rather than a single nucleic acid may improve the efficiency of library construction.

In some embodiments, the method includes removing the binding agent following transfer of the identifying information from the coding tag to the recording tag. For embodiments involving analysis of denatured proteins, polypeptides, and peptides, the bound binding agent and annealed coding tag can be removed following transfer of the identifying information (e.g., primer extension) by using highly denaturing conditions (e.g., 0.1-0.2 N NaOH, 6M Urea, 2.4 M guanidinium isothiocyanate, 95% formamide, etc.).

a. Binding Agents

In certain embodiments, the methods for the macromolecule, e.g., the protein (e.g., polypeptide), analysis assay provided in the present disclosure comprise multiple binding cycles, where the polypeptide is contacted with a plurality of binding agents, and successive binding of binding agents transfers historical binding information in the form of a nucleic acid based coding tag to at least one nucleic acid (e.g., recording tag) associated with the polypeptide. In this way, a historical record containing information about multiple binding events is generated in a nucleic acid format.

The methods described herein use a binding agent capable of binding to the macromolecule, e.g., the polypeptide. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In certain embodiments, a binding agent may be designed to bind covalently. Covalent binding can be designed to be conditional or favored upon binding to the correct moiety. For example, an NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target allows for more stringent washing to be used to remove binding agents that are non-specifically bound, thus increasing the specificity of the assay.

In some embodiments, the binding agent binds to an unmodified or native amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified NTAA, high specificity for a native or unmodified NTAA, or both. In some embodiments, binding agents can be developed through directed evolution of promising affinity scaffolds using phage display.

In certain embodiments, a binding agent may be a selective binding agent. In some embodiments, the binding agent binds to a single amino acid residue, a dipeptide, a tripeptide or a post-translational modification of the polypeptide. In some examples, the binding agent is configured to bind a N-terminal amino acid residue, a C-terminal amino acid residue, or an internal amino acid residue. A binding agent may bind to an N-terminal or C-terminal diamino acid moiety. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In an example of non-selective binding, a binding agent may bind to two or more of the twenty standard amino acids. In some examples, a binding agent binds to an N-terminal amino acid residue, a C-terminal amino acid residue, or an internal amino acid residue.

In some embodiments, the binding agent is partially specific or selective. In some aspects, the binding agent preferentially binds one or more amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and one or more penultimate amino acid. In some cases, the binding agent preferentially binds to one or more specific terminal amino acid(s) and one penultimate amino acid. For example, a binding agent may preferentially bind AA, AC, and AG or a binding agent may preferentially bind AA, CA, and GA. In some specific examples, binding agents with different specificities can share the same coding tag.

In the practice of the methods disclosed herein, the ability of a binding agent to selectively bind a feature or component of a macromolecule, e.g., a polypeptide, need only be sufficient to allow transfer of its coding tag information to the recording tag associated with the polypeptide, transfer of the recording tag information to the coding tag, or transferring of the coding tag information and recording tag information to a di-tag molecule. Thus, selectively need only be relative to the other binding agents to which the polypeptide is exposed. It should also be understood that selectivity of a binding agent need not be absolute to a specific amino acid, but could be selective to a class of amino acids, such as amino acids with nonpolar or non-polar side chains, or with electrically (positively or negatively) charged side chains, or with aromatic side chains, or some specific class or size of side chains, and the like.

In a particular embodiment, the binding agent has a high affinity and high selectivity for the macromolecule, e.g., the polypeptide, of interest. In particular, a high binding affinity with a low off-rate is efficacious for information transfer between the coding tag and recording tag. In certain embodiments, a binding agent has a Kd of <500 nM, <200 nM, <100 nM, <50 nM, <10 nM, <5 nM, <1 nM, <0.5 nM, or <0.1 nM. In a particular embodiment, the binding agent is added to the polypeptide at a concentration >10×, >100×, or >1000× its Kd to drive binding to completion. A detailed discussion of binding kinetics of an antibody to a single protein molecule is described in Chang et al. (Chang, Rissin et al. 2012).

In some embodiments, the binding agent binds to a chemically modified N-terminal amino acid residue or a chemically modified C-terminal amino acid residue. To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer, Thomas et al. 2009); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent may bind to an NTAA, a CTAA, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC2L-A, BC2LCN, BPA, BPL, Calsepa, CGL2, CNL, Con, ConA, DBA, Discoidin, DSA, ECA, EEL, F17AG, Gal1, Gal1-S, Gal2, Gal3, Gal3C-S, Gal7-S, Gal9, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL_I, Malectin, MOA, MPA, MPL, NPA, Orysata, PA-IIL, PA-IL, PALa, PHA-E, PHA-L, PHA-P, PHAE, PHAL, PNA, PPL, PSA, PSL1a, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In some embodiments, a binding agent may bind to a native or unmodified or unlabeled terminal amino acid. In some examples, the binding agent binds to a chemically modified N-terminal amino acid residue or a chemically modified C-terminal amino acid residue. In certain embodiments, a binding agent may bind to a modified or labeled terminal amino acid (e.g., an NTAA that has been functionalized or modified). A modified or labeled NTAA can be one that is functionalized with PITC, 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), N-Acetyl-Isatoic Anhydride, Isatoic Anhydride, 2-Pyridinecarboxaldehyde, 2-Formylphenylboronic acid, 2-Acetylphenylboronic acid, 1-Fluoro-2,4-dinitrobenzene, Succinic anhydride, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N,Ä≤-Bis(pivaloyl)-1H-pyrazole-1-carboxamidine, N,N,Ä≤-Bis(benzyloxycarbonyl)-1H-pyrazole-1-carboxamidine, an acetylating reagent, a guanidinylation reagent, a thioacylation reagent, a thioacetylation reagent, or a thiobenzylation reagent, or a diheterocyclic methanimine reagent. In some examples, the binding agent binds an amino acid labeled by contacting with a reagent or using a method as described in International Patent Publication No. WO 2019/089846. In some cases, the binding agent binds to an amino acid labeled by an amine modifying reagent.

In certain embodiments, a binding agent can be an aptamer (e.g., peptide aptamer, DNA aptamer, or RNA aptamer), an antibody, an anticalin, an ATP-dependent Clp protease adaptor protein (ClpS), an antibody binding fragment, an antibody mimetic, a peptide, a peptidomimetic, a protein, or a polynucleotide (e.g., DNA, RNA, peptide nucleic acid (PNA), a γPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactivity component(s) of an antibody molecule that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab)₂ fragments, single chain antibody fragments (scFv), miniantibodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a macromolecule, e.g., a peptide or a polypeptide, can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, Jayasena, 1999, Clin Chem 45:1628-50; Kusser2000, J. Biotechnol. 74: 27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., reviewed by Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent that selectively binds to a functionalized NTAA can be utilized. For example, the NTAA may be reacted with phenylisothiocyanate (PITC) to form a phenylthiocarbamoyl-NTAA derivative. In this manner, the binding agent may be fashioned to selectively bind both the phenyl group of the phenylthiocarbamoyl moiety as well as the alpha-carbon R group of the NTAA. Use of PITC in this manner allows for subsequent elimination of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction. In yet another embodiment, a binding agent may be an aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In a preferred particular embodiment, the aminopeptidase is a monomeric metallo-protease, such an aminopeptidase activated by zinc (Calcagno and Klein 2016). In another example, a binding agent may selectively bind to an NTAA that is modified with sulfonyl nitrophenol (SNP), e.g., by using 4-sulfonyl-2-nitrofluorobenzene (SNFB).

Other reagents that may be used to functionalize the NTAA include trifluoroethyl isothiocyanate, allyl isothiocyanate, and dimethylaminoazobenzene isothiocyanate, or a reagent as described in International Patent Application No. PCT/US2018/58575.

A binding agent may be engineered for high affinity for a modified NTAA, high specificity for a modified NTAA, or both. In some embodiments, binding agents can be developed through directed evolution of promising affinity scaffolds using phage display.

In another example, highly-selective engineered ClpSs have also been described in the literature. Emili et al. describe the directed evolution of an E. coli ClpS protein via phage display, resulting in four different variants with the ability to selectively bind NTAAs for aspartic acid, arginine, tryptophan, and leucine residues (U.S. Pat. No. 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. See e.g., Schuenemann et al., (2009) EMBO Reports 10(5); Roman-Hernandez et al., (2009) PNAS 106(22):8888-93; Guo et al., (2002) JBC 277(48): 46753-62; Wang et al., (2008) Molecular Cell 32: 406-414. In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of a E. Coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g. PTC, modified-PTC, Cbz, DNP, SNP, acetyl, guanidinyl, diheterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer and Skerra 2012). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin and Charlton 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored. An alternative method for increasing avidity of a binding agent is to include complementary sequences in the coding tag attached to the binding agent and the recording tag associated with the polypeptide.

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binders for use in the methods described herein include: an anticalin, an amino acid tRNA synthetase (aaRS), ClpS, an Affiliₙ®, an Adnectin™, a T cell receptor, a zinc finger protein, a thioredoxin, GST A1-1, DARPin, an affimer, an affitin, an alphabody, an avimer, a Kunitz domain peptide, a monobody, a single domain antibody, EETI-II, HPSTI, intrabody, lipocalin, PHD-finger, V(NAR) LDTI, evibody, Ig(NAR), knottin, maxibody, neocarzinostatin, pVIII, tendamistat, VLR, protein A scaffold, MTI-II, ecotin, GCN4, Im9, kunitz domain, microbody, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2).

As described herein, a binding agent may bind to a post-translationally modified amino acid. Thus, in certain embodiments, an extended nucleic acid associated with the comprises coding tag information relating to amino acid sequence and post-translational modifications of the polypeptide. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA or CTAA). In one example, a peptide is contacted with binding agents for PTM modifications, and associated coding tag information are transferred to the recording tag associated with the immobilized peptide. Once the detection and transfer of coding tag information relating to amino acid modifications is complete, the PTM modifying groups can be removed before detection and transfer of coding tag information for the primary amino acid sequence using N-terminal or C-terminal degradation methods. Thus, resulting extended nucleic acids indicate the presence of post-translational modifications in a peptide sequence, though not the sequential order, along with primary amino acid sequence information.

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended nucleic acids on the recording tag associated with the immobilized peptide indicate the presence and order of post-translational modifications in the context of a primary amino acid sequence.

In certain embodiments, a macromolecule, e.g., a polypeptide, is also contacted with a non-cognate binding agent. As used herein, a non-cognate binding agent is referring to a binding agent that is selective for a different polypeptide feature or component than the particular polypeptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the nt^{h} NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n−1 amino acid of the peptide to the n−1 NTAA (e.g., tyrosine), and the peptide was then contacted with the same three binding agents, the binding agent selective for tyrosine would be second binding agent capable of selectively binding to the n−1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1,000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1,000 nM.

In some embodiments, the ratio between the soluble binding agent molecules and the immobilized macromolecule, e.g., polypeptides, can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher, or any ratio in between the above listed ratios. Higher ratios between the soluble binding agent molecules and the immobilized polypeptide(s) and/or the nucleic acids can be used to drive the binding and/or the coding tag information transfer to completion. This may be particularly useful for detecting and/or analyzing low abundance polypeptides in a sample.

b. Amino Acid Cleavage

In embodiments relating to methods of analyzing peptides or polypeptides using an N-terminal degradation based approach, following contacting and binding of a first binding agent to an n NTAA of a peptide of n amino acids and transfer of the first binding agent's coding tag information to a nucleic acid associated with the peptide, thereby generating a first order extended nucleic acid (e.g., on the recording tag), the n NTAA is eliminated as described herein. Removal of the n labeled NTAA by contacting with an enzyme or chemical reagents converts the n−1 amino acid of the peptide to an N-terminal amino acid, which is referred to herein as an n−1 NTAA. A second binding agent is contacted with the peptide and binds to the n−1 NTAA, and the second binding agent's coding tag information is transferred to the first order extended nucleic acid thereby generating a second order extended nucleic acid (e.g., for generating a concatenated nt^{h} order extended nucleic acid representing the peptide). Elimination of the n−1 labeled NTAA converts the n−2 amino acid of the peptide to an N-terminal amino acid, which is referred to herein as n−2 NTAA. Additional binding, transfer, labeling, and removal, can occur as described above up to n amino acids to generate an n^{th} order extended nucleic acid or n separate extended nucleic acids, which collectively represent the peptide. As used herein, an n “order” when used in reference to a binding agent, coding tag, or extended nucleic acid, refers to the n binding cycle, wherein the binding agent and its associated coding tag is used or the n binding cycle where the extended nucleic acid is created (e.g. on recording tag). In some embodiments, steps including the NTAA in the described exemplary approach can be performed instead with a C terminal amino acid (CTAA).

In some embodiments, contacting of the first binding agent and second binding agent to the polypeptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the polypeptide. In one embodiment, a library of binding agents comprises at least 20 binding agents that selectively bind to the 20 standard, naturally occurring amino acids. In some embodiments, a library of binding agents may comprise binding agents that selectively bind to the modified amino acids.

In other embodiments, the first binding agent and second binding agent, and optionally any further order binding agents, are each contacted with the polypeptide in separate binding cycles, added in sequential order. In certain embodiments, multiple binding agents are used at the same time in parallel. This parallel approach saves time and reduces non-specific binding by non-cognate binding agents to a site that is bound by a cognate binding agent (because the binding agents are in competition).

In certain embodiments relating to analyzing peptides, following binding of a terminal amino acid (N-terminal or C-terminal) by a binding agent and transfer of coding tag information to a recording tag, transfer of recording tag information to a coding tag, transfer of recording tag information and coding tag information to a di-tag construct, the terminal amino acid is removed or cleaved from the peptide to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, cleavage of a terminal amino acid uses a carboxypeptidase, an aminopeptidase, a dipeptidyl peptidase, a dipeptidyl aminopeptidase or a variant, mutant, or modified protein thereof a hydrolase or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc). In some embodiments, an engineered enzyme that catalyzes or reagent that promotes the removal of the PITC-derivatized or other labeled N-terminal amino acid is used. In some aspects, one or more chemical treatments are used to functionalize and/or to eliminate the terminal amino acid of a polypeptide. In some embodiments, the terminal amino acid is removed or eliminated using any of the methods as described in International Patent Publication No. WO 2019/089846 or International Patent Application No. PCT/US20/29969.

Enzymatic cleavage of a NTAA may be accomplished by an aminopeptidase or other peptidases. Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, diheterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. Engineered aminopeptidase mutants that bind to and cleave individual or small groups of labelled (biotinylated) NTAAs have been described (see, PCT Publication No. WO2010/065322). In some cases, the reagent for eliminating the functionalized NTAA is a carboxypeptidase, aminopeptidase, or dipeptidyl peptidase, dipeptidyl aminopeptidase, or variant, mutant, or modified protein thereof.

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Aminopeptidases may be engineered to specifically bind to 20 different NTAAs representing the standard amino acids that are labeled with a specific moiety (e.g., PTC, DNP, SNP, etc.). Control of the stepwise degradation of the N-terminus of the peptide is achieved by using engineered aminopeptidases that are only active (e.g., binding activity or catalytic activity) in the presence of the label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In certain embodiments, the aminopeptidase may be engineered to be non-specific, such that it does not selectively recognize one particular amino acid over another, but rather just recognizes the labeled N-terminus. In yet another embodiment, cyclic cleavage is attained by using an engineered acylpeptide hydrolase (APH) to cleave an acetylated NTAA. In yet another embodiment, amidination (guanidinylation) of the NTAA is employed to enable mild cleavage of the labeled NTAA using NaOH (Hamada, (2016) Bioorg Med Chem Lett 26(7): 1690-1695).

For embodiments relating to CTAA binding agents, methods of cleaving CTAA from peptides are also known in the art. For example, U.S. Pat. No. 6,046,053 discloses a method of reacting the peptide or protein with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In some embodiments, the polypeptide is contacted with one or more additional enzymes to eliminate the NTAA (e.g., a proline aminopeptidase to remove an N-terminal proline, if present). In some embodiments, the enzymes to treat the polypeptides can be used in combination with a chemical or enzymatic methods for removing/eliminating amino acids from the polypeptide. In some cases, enzymes can be provided as a cocktail.

B. Processing and Analysis

In some embodiments, the extended recording tag generated from performing the provided methods comprises information transferred from at least one probe tag and spatial tag. In some embodiments, the extended recording tags may further comprise identifying information from one or more coding tags. In some cases, the extended recording tag comprises information from two or more probe tags and optionally two or more coding tags. In some embodiments, the extended recording tags (or a portion thereof) are amplified prior to determining at least the sequence of the probe tag and spatial tag in the extended recording tag. In some embodiments, the extended recording tags (or a portion thereof) are released prior to determining at least the sequence of the probe tag and spatial tag in the extended recording tag.

Optionally, a spatial sample can be removed from a solid support after macromolecules, e.g., polypeptides, are labeled with the spatial tag and probe tag. Thus, a method of the present disclosure can include a step of removing nucleic acids, macromolecules, cells, tissue or other materials from the spatial sample. Removal of the sample or portions thereof can be performed using any suitable technique and will be dependent on the tissue sample. In some cases, the solid support can be washed with water containing various additives, such as surfactants, detergents, enzymes (e.g., proteases and collagenases), cleavage reagents, or the like, to facilitate removal of the specimen. In some embodiments, the solid support is treated with a solution comprising a proteinase enzyme. In some embodiments, polypeptides are released during or after the specimen is removed from the solid support. In some embodiments, the method includes releasing and/or collecting extended recording tags from the spatial sample. In some embodiments, the extended recording tags released and/or collected contain at least one probe tag and at least one spatial tag.

The length of the final extended nucleic acids (e.g., on the extended recording tag) generated by the methods described herein is dependent upon multiple factors, including the length of the coding tag (e.g., barcode sequence, encoder sequence and spacer), the length of the spatial tag, the length of the probe tag, the length of any other of the nucleic acids (e.g., on the recording tag, optionally including any unique molecular identifier, spacer, universal priming site, barcode, or combinations thereof), the number of transfer cycles performed, and whether coding tags from each binding cycle are transferred to the same extended nucleic acid or to multiple extended nucleic acids.

In some embodiments, an extended recording tag comprises from 5′ to 3′ direction: a universal forward (or 5′) priming sequence, information transferred from the probe tag or spatial tag, and a spacer sequence. In some embodiments, a recording tag comprises from 5′ to 3′ direction: a universal forward (or 5′) priming sequence, information transferred from the probe tag and spatial tag, optionally other barcodes (e.g., sample barcode, partition barcode, compartment barcode, or any combination thereof), and a spacer sequence. In some other embodiments, a recording tag comprises from 5′ to 3′ direction: a universal forward (or 5′) priming sequence, information transferred from the probe tag and spatial tag, optionally other barcodes (e.g., sample barcode, partition barcode, compartment barcode, or any combination thereof), an optional UMI, and a spacer sequence. In some embodiments, information transferred from one or more coding tags is also included.

After the transfer of the final tag information to the extended recording tag from a probe tag, spatial tag, and/or coding tag, the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT-3′-SEQ ID NO:2) or an Illumina P5 primer (5′-AATGATACGGCGACCACCGA-3′-SEQ ID NO:1). The sense or antisense P7 may be appended, depending on strand sense of the nucleic acid to which the identifying information from the coding tag is transferred to. An extended nucleic acid library can be cleaved or amplified directly from the solid support (e.g., beads) and used in traditional next generation sequencing assays and protocols.

In some embodiments, a primer extension reaction is performed on a library of single stranded extended nucleic acids (e.g., extended on the recording tag) to copy complementary strands thereof. In some embodiments, the peptide sequencing assay (e.g., ProteoCode assay), comprises several chemical and enzymatic steps in a cyclical progression. In some cases, one advantage of a single molecule assay is the robustness to inefficiencies in the various cyclical chemical/enzymatic steps. In some embodiments, the use of cycle-specific barcodes present in the coding tag sequence allows an advantage to the assay.

Extended nucleic acids (e.g., extended recording tags) can be processed and analysed using a variety of nucleic acid sequencing methods. In some embodiments, extended recording tags containing the information from one or more probe tags, spatial tags, and any other nucleic acid components are processed and analysed. In some embodiments, the collection of extended recording tags (comprising information from one or more probe tags) can be concatenated. In some embodiments, the extended recording tag(comprising information from one or more probe tags and any other nucleic acid components) can be amplified prior to determining the sequence.

In some embodiments, the recording tag or extended recording tag comprises information from one or more probe tags and spatial tag. In some embodiments, the contained one or more probe tag and spatial tag (e.g., barcodes) is analysed and/or sequenced. In some embodiments, the method includes analyzing the identifying information regarding the binding agent of the macromolecule analysis assay transferred to the recording tag.

Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, sequencing by hybridization, sequencing by synthesis technology (e.g., HiSeg™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™ Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

A library of nucleic acids (e.g., extended nucleic acids) may be amplified in a variety of ways. A library of nucleic acids (e.g., recording tags comprising information from one or more probe tags) undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (˜10 ng), 200 μM dNTP, 1 μM of each forward and reverse amplification primers, 0.5 μl (1U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of nucleic acids (e.g., extended nucleic acids) can undergo target enrichment. In some embodiments, target enrichment can be used to selectively capture or amplify extended nucleic acids representing macromolecules (e.g., polypeptides) of interest from a library of extended nucleic acids before sequencing. In some aspects, target enrichment for protein sequencing is challenging because of the high cost and difficulty in producing highly-specific binding agents for target proteins. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, peptides of interest can be enriched in a sample by enriching their corresponding extended nucleic acids. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of nucleic acids (e.g., extended nucleic acids) is enriched via a hybrid capture-based assay. In a hybrid-capture based assay, the library of extended nucleic acids is hybridized to target-specific oligonucleotides that are labeled with an affinity tag (e.g., biotin). Extended nucleic acids hybridized to the target-specific oligonucleotides are “pulled down” via their affinity tags using an affinity ligand (e.g., streptavidin coated beads), and background (non-specific) extended nucleic acids are washed away. The enriched extended nucleic acids (e.g., extended nucleic acids) are then obtained for positive enrichment (e.g., eluted from the beads). In some embodiments, oligonucleotides complementary to the corresponding extended nucleic acid library representations of peptides of interest can be used in a hybrid capture assay. In some embodiments, sequential rounds or enrichment can also be carried out, with the same or different bait sets.

To enrich the entire length of a polypeptide in a library of extended nucleic acids representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended nucleic acids from a library before sequencing. Examples of undesirable extended nucleic acids that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended nucleic acid library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

Any combination of fractionation, enrichment, and subtraction methods, of the polypeptides before attachment to the solid support and/or of the resulting extended nucleic acid library can economize sequencing reads and improve measurement of low abundance species.

In some embodiments, a library of nucleic acids (e.g., extended nucleic acids) is concatenated by ligation or end-complementary PCR to create a long DNA molecule comprising multiple different extended recorder tags, extended coding tags, or di-tags, respectively (Du et al., (2003) BioTechniques 35:66-72; Muecke et al., (2008) Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, direct single molecule analysis is performed on the nucleic acids (e.g., extended nucleic acids) (see, e.g., Harris et al., (2008) Science 320:106-109). The nucleic acids (e.g., extended nucleic acids) can be analysed directly on the solid support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labeled of decoding oligonucleotides (Gunderson et al., (2004) Genome Res. 14:970-7) can be used to ascertain both the identity and order of the coding tags within the extended nucleic acids (e.g., on the recording tag). In some embodiments, the binding agents may be labeled with cycle-specific coding tags as described above (see also, Gunderson et al., (2004) Genome Res. 14:970-7).

Following sequencing of the nucleic acid libraries (e.g., of extended nucleic acids), the resulting sequences can be collapsed by their UMIs and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

The methods disclosed herein can be used for analysis, including detection, quantitation and/or sequencing, of a plurality of macromolecules simultaneously (multiplexing). Multiplexing as used herein refers to analysis of a plurality of macromolecules (e.g. polypeptides) in the same assay. The plurality of macromolecules can be derived from the same sample or different samples. The plurality of macromolecules can be derived from the same subject or different subjects. The plurality of macromolecules that are analyzed can be different macromolecules, or the same macromolecule derived from different samples. A plurality of macromolecules includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

### V. CORRELATION OF SEQUENCES

The present methods can be used for any suitable purpose including to assess spatial information of one or more macromolecules or associated moieties in a spatial sample.

In some embodiments, the provided methods can be used to assess spatial information of one or more polypeptides in a spatial sample. In still other embodiments, the present methods can be used to assess spatial information or origin of a plurality of macromolecules in a spatial sample. In some embodiments, the identity or at least partial sequence of a plurality of macromolecules, e.g., polypeptides, from the same region is determined.

In some aspects, the transferred information from the probe tag and/or spatial tag to the recording tag links any of the information from extended recording tag to spatial location of the probe tag. In some cases, correlating includes comparing the spatial tag sequence associated with a recording tag to the spatial tag location. In some embodiments, the methods provided thereby allow associating of information from the sequence determined by analyzing the recording tag (e.g., extended recording tag) with spatial information from determining the spatial tag in situ to obtain the spatial location of the spatial tag in the spatial sample.

In some aspects, the transferred information from the probe tag to the recording tag links the information from the molecular probe to the information from the macromolecule analysis assay via sequence of the probe tag. For example, the sequence of the probe tag comprised by the extended recording tag is determined and is correlated to the molecular probe. In some cases, correlating includes comparing the probe tag sequence in an extended recording tag to the probe tag associated with a particular molecular probe to determine the identity of the molecular probe or the detectable label to which it is associated. In some embodiments, the methods provided thereby allow associating of information from the sequence determined by analyzing the recording tag (e.g., extended recording tag) with spatial information determined by assessing, e.g., observing, the detectable label of the molecular probe(s).

In some embodiments, further information from the molecular probe, including characteristics of the target of the molecular probe can be associated with the information on the extended recording tag. For example, any information regarding the sample bound by the molecular probe may also be correlated with the spatial information including tissue/cell phenotype, state, and presence or absence of particular markers.

In some embodiments, any additional information regarding the spatial sample may also be correlated with the information from the optional macromolecule analysis assay. For example, if any histological, cellular, morphological, or anatomical information from any additional staining or imaging is obtained, this information can also be connected to the sequence determined by analyzing the extended recording tag. For example, the other information may be combined by using means of registering the spatial information with other image information, such as fiducial markers that can be used to register and align the images, or by making use of intrinsic information, e.g. detecting a macromolecule in the spatial data set and also in the histological, cellular or other information and correlating the two.

In some embodiments, the method further comprises correlating the sequence of the extended recording tag comprising information transferred from the probe tag and/or spatial with the information of the spatial location of spatial tag determined. In some further embodiments, the provided methods allow determination of the sequence or a partial sequence of the polypeptide and the spatial location of the polypeptide in the spatial sample. In some embodiments, the provided methods allow determination of the identity of macromolecule, e.g., the polypeptide, and its spatial location in the spatial sample. In some embodiments, the provided methods allow determination of the location of the macromolecule in the spatial sample, anatomical, morphological, cellular or subcellular origin of the macromolecule in the spatial sample, information from binding one or more molecular probes, and optionally at least a portion of the sequence of the macromolecule (e.g. polypeptide).

In some instances, the information from the provided methods (spatial information, probe tag information, polypeptide sequence information, any other information on the recording tag, etc.) can be stored, analyzed, and/or determined using a software tool. In some cases, the correlating and associating step of the provided methods may comprise a software tool to determine with some likelihood that each macromolecule at a spatial location of the spatial sample is correlated with a molecular probe. The software may utilize information about the binding characteristics of each molecular probe and/or binding agent. The software could also utilize a listing of some or all spatial locations in which each molecular probe did not bind and use this information about the absence of binding to determine information regarding the macromolecule present at that location. In some embodiments, the software may comprise a database. The database may contain sequences of known proteins in the species from which the sample was obtained or also include related species (e.g. homologs). In some cases, if the species of the sample is unknown then a database of some or all protein sequences may be used. The database may also contain the sequences of any known protein variants and mutant proteins thereof.

In some embodiments, the software may comprise one or more algorithms, such as a machine learning, deep learning, statistical learning, supervised learning, unsupervised learning, clustering, expectation maximization, maximum likelihood estimation, Bayesian inference, linear regression, logistic regression, binary classification, multinomial classification, or other pattern recognition algorithm. For example, the software may perform the one or more algorithms to analyze the information regarding (i) the binding characteristic of each molecular probe used, (ii) information from the database of the macromolecules (e.g. proteins), (iii) information from the recording tag including information contained by the probe tag, spatial tag, and/or information transferred during the macromolecule/polypeptide analysis assay, (iv) the binding characteristics of each binding agent used in the macromolecule/polypeptide analysis assay, (v) information from assessing the spatial tag in situ, and/or (vi) a list of spatial locations, in order to generate or assign a probable identity to each spatial location or associated with each recording tag and/or a confidence (e.g., confidence level and/or confidence interval) for that information. In some aspects, the software performs and uses the information from the correlating and associating step of the methods provided.

In some examples, the provided methods can be used with other methods to identify features of a spatial sample, e.g. optical images of the spatial sample and/or images of histological staining. In some examples, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic reticulum, golgi body, nuclear envelope, and so forth), a compartment of the cell (e.g., cytosol, nuclear fraction, and so forth). The stain may enhance contrast or imaging of intracellular or extracellular structures. In some embodiments, the sample may be stained with haematoxylin and eosin (H&E). By combining other types of information, a richer spatial context for interpreting the protein information may be useful.

### VI. KITS AND ARTICLES OF MANUFACTURE

Provided herein are kits and articles of manufacture comprising components for preparing and analyzing macromolecules (e.g., proteins, polypeptides, or peptides), including spatial information, information from binding the molecular probe, and optionally the sequence or identity of the macromolecule in the sample. In some examples, the information includes spatial information regarding the protein and the sequence or identity of the protein. The kits and articles of manufacture may include any one or more of the reagents and components used in the methods described in Sections I-IV. In some embodiments, the kits optionally include instructions for use. In some embodiments, the kits comprise one or more of the following components: spatial probe(s), spatial tag(s), molecular probe(s), probe tag(s), reagent(s) for sequencing, recoding tag(s), reagent(s) for attaching the recording tag, reagent(s) for transferring information from the probe tag to the recording tag, reagent(s) for transferring information from the spatial tag to the recording tag, binding agent(s), reagent(s) for transferring identifying information from the coding tag to the recording tag, sequencing reagent(s), and/or solid support(s), as described in the methods for analyzing the macromolecules (e.g., proteins, polypeptides, or peptides), enzyme(s), buffer(s), sample processing reagent(s) (fixation and permeabilization reagent(s) and buffer(s).

In some embodiments, the kits also include other component(s) for treating the macromolecules (e.g., proteins, polypeptides, or peptides) and analysis of the same, including other reagent(s) for polypeptide analysis. In one aspect, provided herein are components used to prepare a reaction mixture. In preferred embodiments, the reaction mixture is a solution. In preferred embodiments, the reaction mixture includes one or more of the following: molecular probe(s) comprising a probe tag (and optional detectable label), recording tag, solid support(s), binding agent(s) with associated coding tag(s), one or more reagent(s) for attaching a tag to a macromolecule, reagent(s) for transferring information from the probe tag to the recording tag, enzyme(s), buffer(s), sample processing reagent(s) (fixation and permeabilization reagent(s) and buffer(s)).

In another aspect, disclosed herein is a kit for analyzing a polypeptide, comprising: a library of binding agents, wherein each binding agent comprises a binding moiety and a coding tag comprising identifying information regarding the binding moiety, wherein the binding moiety is capable of binding to one or more N-terminal, internal, or C-terminal amino acids of the fragment, or capable of binding to the one or more N-terminal, internal, or C-terminal amino acids modified by a functionalizing reagent.

In some embodiments, the kits and articles of manufacture comprise molecular probes as described in Section II.B and III.B and optionally spatial probes as described in Section II.C. The molecular probes may be provided as a library of molecular probes. The spatial probes may also be provided as a plurality of spatial probes. The molecular probes and/or spatial probes may be combined or provided in separate containers containing individual or subsets of the probes. In some embodiments, each of the molecular probes are associated with a probe tag. Optionally, each or some of the molecular probes may be associated with a detectable label. Also included are reagent(s) for transferring identifying information from the probe tag and spatial tag to the recording tag.

In some embodiments, the kits and articles of manufacture further comprise a plurality of barcodes. The barcode may include a compartment barcode, a partition barcode, a sample barcode, a fraction barcode, or any combination thereof. In some cases, the barcode comprises a unique molecule identifier (UMI). In some examples, the barcode comprises a peptide, DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof. In some embodiments, the barcodes are configured to attach the macromolecules, e.g., the proteins, in the sample or to attach to nucleic components associated with the macromolecules, e.g., the proteins. In some examples, additional linkers for attaching barcodes may be provided in the kit.

In some embodiments, the kit further comprises reagents for treating the macromolecules, e.g., the proteins. Any combination of fractionation, enrichment, and subtraction methods, of the macromolecules, e.g., the proteins, may be performed. For example, the reagent may be used to fragment or digest the macromolecules, e.g., the proteins. In some cases, the kit comprises reagents and components to fractionate, isolate, subtract, enrich the macromolecules, e.g., the proteins. In some examples, the kits further comprises a protease such as trypsin, LysN, or LysC.

In some embodiments, the kit also comprises one or more buffers or reaction fluids necessary for any of the desired reaction to occur. Buffers including wash buffers, reaction buffers, and binding buffers, elution buffers and the like are known to those or ordinary skill in the arts. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed.

In some embodiments, the kit includes one or more reagents for nucleic acid sequence analysis. In some examples, the reagent for sequence analysis is for use in sequencing by synthesis, sequencing by ligation, single molecule sequencing, single molecule fluorescent sequencing, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In some embodiments, the kits or articles of manufacture may further comprise instruction(s) on the methods and uses described herein. In some embodiments, the instructions are directed to methods of analyzing the macromolecules (e.g., proteins, polypeptides, or peptides). The kits described herein may also include other materials desirable from a commercial and user standpoint, including other buffers, diluents, filters, syringes, and package inserts with instructions for performing any methods described herein.

Any of the above-mentioned kit components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

### VII. EXEMPLARY EMBODIMENTS

Among the provided embodiments are:

1. A method of analyzing a macromolecule comprising:

(a) providing a spatial sample comprising a macromolecule associated with a recording tag;

(b1) providing a spatial probe comprising a spatial tag to the spatial sample;

(b2) assessing the spatial tag in situ to obtain the spatial location of the spatial tag in the spatial sample;

(b3) extending the recording tag by transferring information from the spatial tag in the spatial probe to the recording tag;

(c1) binding a molecular probe comprising a probe tag to the macromolecule or a moiety in proximity to the macromolecule in the spatial sample;

(c2) extending the recording tag by transferring information from the probe tag in the molecular probe to the recording tag, wherein transferring information from the spatial tag and/or probe tag to the recording tag generates an extended recording tag;

(d) determining at least the sequence of the probe tag and spatial tag in the extended recording tag; and

(e) correlating the sequence of the spatial tag determined in step (d) with the spatial tag assessed in step (b2);

thereby associating information from the sequence of the extended recording tag or a portion thereof, e.g., the information from the spatial tag and/or probe tag, determined in step (d) with the spatial location of the spatial probe assessed in step (b2).

2. The method of embodiment 1, wherein the method is for analyzing a plurality of macromolecules in the spatial sample.

3. The method of embodiment 1 or embodiment 2, wherein the macromolecule is a protein.

4. The method of any one of embodiments 1-3, wherein the macromolecule is a polypeptide or a peptide.

5. The method of any one of embodiments 1-4, wherein the method comprises binding a plurality of molecular probes to the spatial sample.

6. The method of any one of embodiments 1-5, wherein the method comprises providing a plurality of spatial probes to the spatial sample.

7. The method of any one of embodiments 1-6, further comprising repeating step (c1) and step (c2) sequentially two or more times.

8. The method of embodiment 6, further comprising removing the molecular probe from the spatial sample prior to repeating step (c1).

9. The method of any one of embodiments 1-8, wherein the spatial probe comprises a support and a spatial tag comprising a nucleic acid.

10. The method of embodiment 9, wherein the support comprises a bead or a nanoparticle.

11. The method of embodiment 10, wherein the bead or nanoparticle ranges between about 0.1 μm to about 100 μm, between about 0.1 μm to about 50 μm, between about 10 μm to about 50 μm, between about 5 μm to about 10 μm, between about 0.5 μm to about 100 μm, between about 0.5 μm to about 50 μm, between about 0.5 μm to about 10 μm, between about 0.5 μm to about 5 μm, or between about 0.5 μm to about 1 μm in diameter.

12. The method of any one of embodiments 1-11, wherein the spatial probe comprises a barcoded bead.

13. The method of any one of embodiments 6-12, wherein the spatial probes are randomly distributed on the spatial sample.

14. The method of any one of embodiments 9-13, wherein the spatial tag is attached to the support with a cleavable linker.

15. The method of any one of embodiments 1-14, wherein the spatial tag comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof.

16. The method of any one of embodiments 1-15, wherein the spatial tag comprises a universal priming site.

17. The method of any one of embodiments 1-16, wherein the spatial tag comprises a barcode.

18. The method of embodiment 17, wherein the spatial probe comprises a plurality of barcodes.

19. The method of embodiment 18, wherein the spatial probe comprises two or more copies of the same barcodes.

20. The method of any one of embodiments 1-19, wherein the spatial tag comprises a spacer.

21. The method of any one of embodiments 1-20, wherein the spatial tag comprises a sequence complementary to the recording tag or a portion thereof.

22. The method of any one of embodiments 1-21, wherein the spatial probe non-specifically associates with the spatial sample.

23. The method of embodiment 22, wherein the spatial probe associates with the spatial sample via charge interaction, DNA hybridization, and/or reversible chemical coupling.

24. The method of any one of embodiments 1-23, wherein performing step (b2) comprises obtaining an image of the spatial sample or a portion thereof.

25. The method of embodiment 24, wherein two or more images of the spatial sample or a portion thereof are obtained.

26. The method of embodiment 25, further comprising comparing, aligning, and/or overlaying two or more images.

27. The method of any one of embodiments 1-26, wherein performing step (b2) comprises using a microscope.

28. The method of embodiment 27, wherein the microscope is a fluorescence microscope.

29. The method of any one of embodiments 1-28, wherein the spatial tag is assessed in step (b2) using a decoder, wherein the decoder comprises a detectable label and a sequence complementary to the spatial tag or a portion thereof.

30. The method of embodiment 29, wherein two or more decoders are used to detect one or more of the spatial tags.

31. The method of embodiment 29 or embodiment 30, wherein the detectable label comprises a radioisotope, a fluorescent label, a colorimetric label or an enzyme-substrate label.

32. The method of embodiments 1-23, wherein step (b2) comprises sequencing by ligation, single molecule sequencing, single molecule fluorescent sequencing, or sequencing by probe detection.

33. The method of any one of embodiments 1-32, wherein the spatial tag is transferred to the recording tag by primer extension or ligation.

34. The method of any one of embodiments 1-33, wherein extending the recording tag by transferring information from the spatial tag to the recording tag comprises contacting the spatial sample with a polymerase and a nucleotide mix, thereby adding one or more nucleotides to the recording tag.

35. The method of any one of embodiments 1-34, wherein the molecular probe comprises a nucleic acid, a polypeptide, a small molecule, or any combination thereof.

36. The method of embodiment 35, wherein the molecular probe comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

37. The method of any one of embodiments 1-36, wherein the molecular probe comprises a targeting moiety capable of specific binding.

38. The method of embodiment 37, wherein the targeting moiety is configured to bind to a nucleic acid, a carbohydrate, a lipid, a polypeptide, a post-translational modification of a polypeptide, or any combination thereof.

39. The method of embodiment 37 or embodiment 38, wherein the targeting moiety is a protein-specific targeting moiety.

40. The method of embodiment 37 or embodiment 38, wherein the targeting moiety is an epitope-specific targeting moiety.

41. The method of embodiment 37 or embodiment 38, wherein the targeting moiety is a nucleic acid-specific targeting moiety.

42. The method of any one of embodiments 37-41, wherein the targeting moiety is configured to bind to a cell surface marker.

43. The method of any one of embodiments 1-42, wherein the binding in step (c1) comprises chemical binding, covalent binding, and/or reversible binding.

44. The method of any one of embodiments 1-43, wherein the probe tag comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof.

45. The method of any one of embodiments 1-44, wherein the probe tag comprises a universal priming site.

46. The method of any one of embodiments 1-45, wherein the probe tag comprises a barcode.

47. The method of any one of embodiments 1-46, wherein the probe tag comprises a spacer.

48. The method of any one of embodiments 1-47, wherein the probe tag comprises a complementary sequence to the recording tag or a portion thereof.

49. The method of any one of embodiments 1-48, wherein the probe tag is transferred to the recording tag by primer extension or ligation.

50. The method of any one of embodiments 1-49, wherein information from the probe tag is transferred to a recording tag in the vicinity of the associated molecular probe.

51. The method of any one of embodiments 1-50, wherein extending the recording tag by transferring information from the probe tag to the recording tag comprises contacting the spatial sample with a polymerase and a nucleotide mix, thereby adding one or more nucleotides to the recording tag.

52. The method of any one of embodiments 1-51, wherein step (c2) comprises transferring information from the probe tag directly or indirectly via a copy of the probe tag to the recording tag.

53. The method of any one of embodiments 1-52, wherein step (c2) comprises transferring the information from one probe tag to two or more recording tags.

54. The method of any one of embodiments 1-53, wherein the probe tag is amplified prior to step (c2).

55. The method of embodiment 54, wherein the amplification is linear amplification.

56. The method of embodiment 55, wherein amplification of the probe tag is performed using a RNA polymerase.

57. The method of embodiments 56, wherein transferring information of the probe tag to the recording tag is performed using reverse transcription.

58. The method of any one of embodiments 1-57, further comprising performing a macromolecule analysis assay.

59. The method of embodiment 58, wherein the macromolecule analysis assay is a polypeptide analysis assay.

60. The method of embodiment 58 or embodiment 59, wherein the macromolecule analysis assay is performed in situ.

61. The method of any one of embodiments 58-60, further comprising releasing the macromolecule associated with the recording tag from the spatial sample prior to performing the macromolecule analysis assay.

62. The method of any one of embodiments 58-61, further comprising collecting the macromolecule associated with the recording tag prior to performing the macromolecule analysis assay.

63. The method of any one of embodiments 58-62, wherein the macromolecule is coupled directly or indirectly to a solid support prior to performing the macromolecule analysis assay.

64. The method of any one of embodiments 58-63, wherein the macromolecule analysis assay comprises:

contacting the macromolecule with a binding agent capable of binding to the macromolecule, wherein the binding agent comprises a coding tag with identifying information regarding the binding agent; and extending the recording tag associated with the macromolecule by transferring the information of the coding tag to the recording tag.

65. The method of embodiment 64, further comprising repeating one or more times: contacting the macromolecule with an additional binding agent capable of binding to the macromolecule, wherein the additional binding agent comprises a coding tag with identifying information regarding the additional binding agent; and extending the recording tag associated with the macromolecule by transferring the identifying information of the coding tag regarding the additional binding agent to the recording tag.

66. The method of any one of embodiments 58-65, wherein transferring the identifying information of the coding tag to the recording tag is by primer extension or ligation.

67. The method of any one of embodiments 58-65, wherein transferring the identifying information of the coding tag to the recording tag is mediated by a DNA polymerase.

68. The method of any one of embodiments 58-65, wherein transferring the identifying information of the coding tag to the recording tag is mediated by a DNA ligase.

69. The method of any one of embodiments 58-68, wherein the coding tag further comprises a spacer, a binding cycle specific sequence, a unique molecular identifier, a universal priming site, or any combination thereof 70. The method of embodiment 69, wherein the coding tag comprises a spacer at its 3′-terminus.

71. The method of any one of embodiments 58-70, wherein the binding agent and the coding tag are joined by a linker. 72. The method of any one of embodiments 58-71, wherein the binding agent is a polypeptide or protein.

73. The method of embodiment 72, wherein the binding agent is a modified aminopeptidase, a modified amino acyl tRNA synthetase, a modified anticalin, or an antibody or a binding fragment thereof.

74. The method of any one of embodiments 58-73, wherein the binding agent binds to a single amino acid residue, a dipeptide, a tripeptide or a post-translational modification of the peptide.

75. The method of embodiment 74, wherein the binding agent binds to an N-terminal amino acid residue, a C-terminal amino acid residue, or an internal amino acid residue.

76. The method of embodiment 74, wherein the binding agent binds to a chemically modified N-terminal amino acid residue or a chemically modified C-terminal amino acid residue.

77. The method of embodiment 75 or embodiment 76, wherein the binding agent binds to the N-terminal amino acid residue and the N-terminal amino acid residue is cleaved after transferring the information of the coding tag to the recording tag.

78. The method of embodiment 75 or embodiment 76, wherein the binding agent binds to the C-terminal amino acid residue and the C-terminal amino acid residue is cleaved after transferring the information of the coding tag to the recording tag.

79. The method of embodiments 1-78, wherein the extended recording tag comprises information from one or more probe tags, one or more spatial tags, and optionally one or more coding tags.

80. The method of any one of embodiments 1-79, wherein the extended recording tag comprises information from two or more probe tags, two or more spatial tags, and optionally two or more coding tags.

81. The method of any one of embodiments 1-80, wherein the extended recording tag is amplified prior to step (d).

82. The method of any one of embodiments 1-80, wherein the extended recording tag is released from the spatial sample prior to step (d).

83. The method of any one of embodiments 58-82, further comprising determining at least a portion of the sequence of the macromolecule and associating with its spatial location assessed in step (b2).

84. The method of embodiment 83, wherein step (d) comprises sequencing by synthesis, sequencing by ligation, sequencing by hybridization, polony sequencing, ion semiconductor sequencing, pyrosequencing, single molecule real-time sequencing, nanopore-based sequencing, or direct imaging of DNA using advanced microscopy.

85. The method of any one of embodiments 1-84, wherein the spatial sample comprises a plurality of macromolecules, e.g., polypeptides.

86. The method of any one of embodiments 1-85, wherein the spatial sample is provided on a solid support.

87. The method of any one of embodiments 1-86, wherein the spatial sample comprises a plurality of cells deposited on a surface.

88. The method of any one of embodiments 1-87, wherein the spatial sample comprises a tissue sample.

89. The method of any one of embodiments 1-88, wherein the spatial sample is a formalin-fixed, paraffin-embedded (FFPE) section or a cell spread.

90. The method of any one of embodiments 1-89, further comprising treating the spatial sample with a fixing and/or cross-linking agent.

91. The method of any one of embodiments 1-90, further comprising treating the spatial sample with a permeabilizing agent.

92. The method of embodiment 90 or embodiment 91, wherein treating the spatial sample with the fixing, cross-linking, and/or permeabilizing reagent is performed prior to step (b1) and/or step (c).

93. The method of any one of embodiments 58-92, wherein the polypeptide is fragmented prior to performing the polypeptide analysis assay.

94. The method of embodiment 93, wherein the fragmenting is performed by contacting the polypeptide(s) with a protease.

95. The method of embodiment 94, wherein the protease is trypsin, LysN, or LysC.

96. The method of any one of embodiments 63-95, wherein the solid support comprises a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.

97. The method of embodiment 96, wherein the solid support comprises a polystyrene bead, a polyacrylate bead, a cellulose bead, a dextran bead, a polymer bead, an agarose bead, an acrylamide bead, a solid core bead, a porous bead, a paramagnetic bead, glass bead, or a controlled pore bead, or any combination thereof.

98. The method of any one of embodiments 1-97, wherein the recording tag comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof.

99. The method of any one of embodiments 1-98, wherein step (a) comprises providing the spatial sample with a plurality of recording tags.

100. The method of any one of embodiments 1-99, wherein the recording tag is comprised in a matrix applied to the spatial sample.

101. The method of any one of embodiments 1-99, wherein the recording tag is associated directly or indirectly to the macromolecule.

102. The method of any one of embodiments 1-99, wherein the macromolecule is coupled directly or indirectly to the recording tag.

103. The method of any one of embodiments 1-102, wherein the recording tag, spatial tag, and/or probe tag comprises a unique molecular identifier (UMI).

104. The method of any one of embodiments 1-103, wherein the recording tag comprises a compartment tag.

105. The method of any one of embodiments 1-104, wherein the recording tag comprises a universal priming site.

106. The method of any one of embodiments 1-105, wherein the recording tag comprises a spacer polymer.

107. The method of embodiment 106, wherein the spacer is at the 3′-terminus of the recording tag.

108. The method of any one of embodiments 1-107, wherein:

step (a) is performed prior to steps (b1), (b2), (b3), (c1), (c2), (d), and (e);

step (b1) is performed prior to steps (b2), (d), and (e);

steps (e1) and (c2) is performed prior to steps (d) and step (e);

steps (e1) and (c2) is performed prior to or after steps (b1), (b2), and/or (b3);

step (d) is performed prior to step (e); and/or

step (e) is performed after steps (a) (b1), (b2), (b3), (e1), (c2), and (d).

109. The method of any one of embodiments 1-108, wherein steps (e1) and (c2) are sequentially repeated two or more times prior to performing steps (d) and (e).

110. The method of any one of embodiments 1-109 wherein steps (e1) and (c2) are performed prior to steps (b1), (b2), and (b3).

111. The method of any one of embodiments 1-110, wherein step (b2) is performed after step

112. The method of any one of embodiments 1-111, wherein step (b2) is performed prior to or after step (b3).

113. The method of any one of embodiments 1-112, wherein:

steps (a), (c1), (c2), (b1), (b2), (b3), (d), and (e) occur in sequential order.

114. The method of any one of embodiments 1-113, wherein:

the molecular probe is removed prior to providing a spatial probe to the spatial sample; or

the spatial probe is removed from the sample prior to binding the sample with a molecular probe.

115. The method of any one of embodiments 58-114, the macromolecule analysis assay is performed before step (d) and step (e).

116. A method of analyzing a macromolecule comprising:

(a) providing a spatial sample comprising a macromolecule with a recording tag;

(b) binding a molecular probe comprising a detectable label and a probe tag to the macromolecule or a moiety in proximity to the macromolecule in the spatial sample;

(c) transferring information from the probe tag in the molecular probe to the recording tag to generate an extended recording tag;

(d) assessing, e.g., observing, the detectable label to obtain spatial information of the molecular probe;

(e) determining at least the sequence of the probe tag in the extended recording tag; and correlating the sequence of the probe tag determined in step (e) with the molecular probe;

thereby associating information from the sequence determined in step (e) with its spatial information determined in step (d).

117. The method of embodiment 116, wherein the macromolecule is a protein.

118. The method of embodiment 116, wherein the macromolecule is a polypeptide or a peptide.

119. The method of any one of embodiments 116-118, wherein the method comprises binding a plurality of the molecular probes to the spatial sample.

120. The method of embodiment 119, wherein two or more probes are associated with the same detectable label.

121. The method of embodiment 119, wherein each molecular probe in the plurality of molecular probes is associated with a unique detectable label.

122. The method of any one of embodiments 116-121, further comprising repeating step (b) and step (c) sequentially two or more times.

123. The method of embodiment 122, further comprising repeating step (d) two or more times.

124. The method of embodiment 122 or embodiment 123, further comprising removing the molecular probe from the spatial sample prior to repeating step (b).

125. The method of embodiment 112 or embodiment 123, further comprising inactivating the detectable label after assessing, e.g., observing the detectable label.

126. The method of any one of embodiments 116-125, wherein the molecular probe comprises a nucleic acid, a polypeptide, a small molecule, or any combination thereof.

127. The method of any one of embodiments 116-126, wherein the molecular probe comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

128. The method of any one of embodiments 116-127, wherein the molecular probe comprises a targeting moiety capable of specific binding.

129. The method of embodiment 128, wherein the targeting moiety is configured to bind a nucleic acid, a carbohydrate, a lipid, a polypeptide, a post-translational modification of a polypeptide, or any combination thereof.

130. The method of embodiment 128 or embodiment 129, wherein targeting moiety is a protein-specific targeting moiety.

131. The method of embodiment 128 or embodiment 129, wherein targeting moiety is an epitope-specific targeting moiety.

132. The method of embodiment 128 or embodiment 129, wherein the targeting moiety is a nucleic acid-specific targeting moiety.

133. The method of any one of embodiments 128-132, wherein targeting moiety is configured to bind a cell surface marker.

134. The method of any one of embodiments 128-133, wherein the binding in step (b) includes chemical binding, covalent binding, and/or reversible binding.

135. The method of any one of embodiments 116-134, wherein the detectable label comprises a radioisotope, a fluorescent label, a colorimetric label or an enzyme-substrate label.

136. The method of any one of embodiments 116-135, wherein assessing, e.g., observing, the detectable label comprises obtaining a digital image of the spatial sample or a portion thereof.

137. The method of embodiment 136, wherein two or more digital images of the spatial sample are obtained.

138. The method of embodiment 137, wherein the two or more digital images provide combinatorial spatial information of the plurality of molecular probes.

139. The method of embodiment 137 or embodiment 138, further comprising comparing, aligning, and/or overlaying at least two of the images.

140. The method of any one of embodiments 116-139, further comprising inactivating the detectable label after assessing, e.g., observing, the detectable label.

141. The method of any one of embodiments 116-140, wherein assessing, e.g., observing, the detectable label is performed using a microscope.

142. The method of embodiment 141, wherein assessing, e.g., observing, the detectable label is performed using a fluorescence microscope.

143. The method of any one of embodiments 116-142, wherein information from the the probe tag is transferred to the recording tag by primer extension or ligation.

144. The method of embodiment 143, wherein transferring information from the probe tag to the recording tag comprises contacting the spatial sample with a polymerase and a nucleotide mix, thereby adding one or more nucleotides to the recording tag.

145. The method of any one of embodiments 116-144, wherein information from the probe tag is transferred to a recording tag in the vicinity of the probe tag.

146. The method of any one of embodiments 116-145, wherein step (c) comprises transferring information from the probe tag directly or indirectly via a copy of the probe tag to the recording tag.

147. The method of any one of embodiments 116-146, wherein step (c) comprises transferring the information from one probe tag to two or more recording tags.

148. The method of any one of embodiments 116-147, wherein the probe tag is amplified prior to step (c).

149. The method of embodiment 148, wherein amplification of the probe tag is performed using a RNA polymerase.

150. The method of embodiment 148, wherein the amplification is linear amplification.

151. The method of embodiments 149 or embodiment 150, wherein transferring information from the probe tag to the recording tag is performed using reverse transcription.

152. The method of any one of embodiments 116-151, wherein step (a) comprises providing the spatial sample with a plurality of recording tags.

153. The method of any one of embodiments 116-152, wherein the recording tag is comprised in a matrix applied to the spatial sample.

154. The method of any one of embodiments 116-152, wherein the recording tag is associated directly or indirectly to the macromolecule.

155. The method of any one of embodiments 116-151 and 154, wherein the macromolecule is coupled directly or indirectly to the recording tag.

156. The method of any one of embodiments 116-155, further comprising performing a macromolecule analysis assay.

157. The method of embodiment 156, wherein the macromolecule analysis assay is a polypeptide analysis assay.

158. The method of embodiment 156 or embodiment 157, wherein the macromolecule analysis assay is performed in situ.

159. The method of any one of embodiments 156-158, further comprising releasing the macromolecule associated with the recording tag from the spatial sample prior to performing the macromolecule analysis assay.

160. The method of any one of embodiments 156-159, further comprising collecting the macromolecule associated with the recording tag prior to performing the macromolecule analysis assay.

161. The method of any one of embodiments 156-160, wherein the macromolecule is coupled directly or indirectly to a solid support prior to performing the macromolecule analysis assay.

162. The method of any one of embodiments 156-161, wherein the macromolecule analysis assay comprises:

contacting the macromolecule with a binding agent capable of binding to the macromolecule, wherein the binding agent comprises a coding tag with identifying information regarding the binding agent; and

transferring the information of the coding tag to the recording tag to generate the extended recording tag.

163. The method of embodiment 162, further comprising repeating one or more times:

contacting the macromolecule with an additional binding agent capable of binding to the macromolecule, wherein the additional binding agent comprises a coding tag with identifying information regarding the additional binding agent; and

transferring the identifying information of the coding tag regarding the additional binding agent to the extended recording tag.

164. The method of embodiment 162 or embodiment 163, wherein transferring the identifying information of the coding tag to the recording tag is mediated by a DNA ligase.

165. The method of embodiment 162 or embodiment 163, wherein transferring the identifying information of the coding tag to the recording tag is mediated by a DNA polymerase.

166. The method of embodiment 162 or embodiment 163, wherein transferring the identifying information of the coding tag to the recording tag is mediated by chemical ligation.

167. The method of any one of embodiments 162-166, wherein the coding tag further comprises a spacer, a binding cycle specific sequence, a unique molecular identifier, a universal priming site, or any combination thereof.

168. The method of embodiment 167, wherein the coding tag comprises a spacer at its 3′-terminus.

169. The method of any one of embodiments 162-168, wherein the binding agent and the coding tag are joined by a linker.

170. The method of any one of embodiments 162-169, wherein the binding agent is a polypeptide or protein.

171. The method of embodiment 170, wherein the binding agent is a modified aminopeptidase, a modified amino acyl tRNA synthetase, a modified anticalin, or an antibody or a binding fragment thereof.

172. The method of any one of embodiments 162-171, wherein the binding agent binds to a single amino acid residue, a dipeptide, a tripeptide or a post-translational modification of the polypeptide.

173. The method of embodiment 172, wherein the binding agent binds to an N-terminal amino acid residue, a C-terminal amino acid residue, or an internal amino acid residue.

174. The method of embodiment 172, wherein the binding agent binds to a chemically modified N-terminal amino acid residue or a chemically modified C-terminal amino acid residue.

175. The method of embodiment 173 or embodiment 174, wherein the binding agent binds to the N-terminal amino acid residue and the N-terminal amino acid residue is cleaved after transferring the information of the coding tag to the recording tag.

176. The method of embodiment 173 or embodiment 174, wherein the binding agent binds to the C-terminal amino acid residue and the C-terminal amino acid residue is cleaved after transferring the information of the coding tag to the recording tag.

177. The method of any one of embodiments 162-176, wherein the extended recording tag comprises information from one or more probe tags and one or more coding tags.

178. The method of any one of embodiments 162-176, wherein the extended recording tag comprises information from two or more probe tags and two or more coding tags.

179. The method of any one of embodiments 116-178, wherein the extended recording tag is amplified prior to step (e).

180. The method of any one of embodiments 116-179, wherein step (e) comprises sequencing by synthesis, sequencing by ligation, sequencing by hybridization, polony sequencing, ion semiconductor sequencing, pyrosequencing, single molecule real-time sequencing, nanopore-based sequencing, or direct imaging of DNA using advanced microscopy.

181. The method of any one of embodiments 116-180, wherein the spatial sample comprises a plurality of the macromolecules, e.g., the polypeptides.

182. The method of any one of embodiments 116-181, wherein the spatial sample is provided on a solid support.

183. The method of embodiment 182, wherein the spatial sample comprises a plurality of cells deposited on a surface.

184. The method of any one of embodiments 116-182, wherein the spatial sample comprises a tissue sample.

185. The method of any one of embodiments 116-182, wherein the spatial sample is a formalin-fixed, paraffin-embedded (FFPE) section or a cell spread.

186. The method of any one of embodiments 156-185, further comprising determining at least a portion of the sequence of the macromolecule and associating with its spatial location determined in step (d).

187. The method of any one of embodiments 116-185, further comprising treating the spatial sample with a fixing agent, a cross-linking agent, and or a permeabilizing agent.

188. The method of embodiment 187, wherein the fixing, cross-linking, and/or permeabilizing the spatial sample is performed prior to step (b).

189. The method of any one of embodiments 157-188, wherein the polypeptide is fragmented prior to performing the polypeptide analysis assay.

190. The method of embodiment 189, wherein the fragmenting is performed by contacting the polypeptide(s) with a protease.

191. The method of embodiment 190, wherein the protease is trypsin, LysN, or LysC.

192. The method of any one of embodiments 161-191, wherein the solid support comprises a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.

193. The method of embodiment 192, wherein the solid support comprises a polystyrene bead, a polyacrylate bead, a cellulose bead, a dextran bead, a polymer bead, an agarose bead, an acrylamide bead, a solid core bead, a porous bead, a paramagnetic bead, glass bead, or a controlled pore bead, or any combinations thereof.

194. The method of any one of embodiments 116-193, wherein the probe tag comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof.

195. The method of any one of embodiments 116-194, wherein the probe tag comprises a universal priming site.

196. The method of any one of embodiments 116-195, wherein the probe tag comprises a barcode.

197. The method of any one of embodiments 116-196, wherein the probe tag comprises a spacer.

198. The method of any one of embodiments 116-197, wherein the recording tag comprises a DNA molecule, DNA with pseudo-complementary bases, an RNA molecule, a BNA molecule, an XNA molecule, a LNA molecule, a PNA molecule, a γPNA molecule, a non-nucleic acid sequenceable polymer, e.g., a polysaccharide, a polypeptide, a peptide, or a polyamide, or a combination thereof.

199. The method of any one of embodiments 116-198, wherein the recording tag and/or probe tag comprises a unique molecular identifier (UMI).

200. The method of any one of embodiments 116-199, wherein the recording tag comprises a compartment tag.

201. The method of any one of embodiments 116-200, wherein the recording tag comprises a universal priming site.

202. The method of any one of embodiments 116-200, wherein the recording tag comprises a spacer polymer.

203. The method of embodiment 202, wherein the spacer is at the 3′-terminus of the recording tag.

204. The method of any one of embodiments 116-203, wherein:

step (a) is performed prior to steps (b), (c), (d), (e), and (f);

step (b) is performed prior to steps (c), (d), (e), and (f);

step (c) is performed prior to or after step (d);

step (c) is performed before steps (e), and (f);

step (d) is performed before steps (e), and (f);

step (e) is performed after steps (a) (b), (c), and (d); and/or

step (e) is performed before steps (0.

205. The method of any one of embodiments 116-203, wherein:

steps (a), (b), (c), (d), (e), and (f) occur in sequential order; or

steps (a), (b), (d), (c), (e), and (f) occur in sequential order.

206. The method of embodiment 205, wherein steps (b), (c), and (d) are sequentially repeated two or more times prior to performing steps (e) and (f).

207. The method of embodiment 205, wherein steps (b), (d), and (c) are sequentially repeated two or more times prior to performing steps (e) and (f).

208. The method of any one of embodiments 156-207, wherein the macromolecule analysis assay is performed prior to step (e) and step (f).

209. The method of any one of embodiments 156-208, wherein the macromolecule analysis assay is performed after steps (a), (b), (c), and (d).

210. A method of analyzing a macromolecule comprising:

(a) providing a spatial sample comprising a macromolecule associated with a recording tag;

(b) assessing the spatial location of the macromolecule in the spatial sample in situ;

(c1) binding a molecular probe comprising and a probe tag to the macromolecule or a moiety in proximity to the macromolecule in the spatial sample;

(c2) extending the recording tag by transferring information from the probe tag in the molecular probe to the recording tag, wherein transferring information from the probe tag to the recording tag generates an extended recording tag;

(d) determining at least the sequence of the probe tag in the extended recording tag; and

(e) correlating the sequence of the probe tag determined in step (d) with the molecular probe and/or spatial location assessed in step (b);

thereby associating information from the sequence of the extended recording tag or a portion thereof determined in step (d) with the spatial location assessed in step (b).

211. The method of embodiment 210, wherein the macromolecule in step (a) is provided with a spatial tag associated directly or indirectly with the recording tag.

212. The method of embodiment 211, wherein the recording tag comprises a UMI.

213. The method of any one of embodiments 210-212, wherein step (b) comprises analyzing the spatial tag in situ.

214. The method of embodiment 213, wherein the spatial tag sequence is analyzed using a microscope-based method.

215. The method of embodiment 214, wherein the microscope-based method is multiplexed.

216. The method of any one of embodiments 211-215, wherein the spatial tag sequence is analyzed by sequencing.

217. The method of embodiment 216, wherein the sequencing comprises sequencing by ligation, single molecule sequencing, single molecule fluorescent sequencing, or sequencing by probe detection.

218. The method of embodiment 210, wherein step (b) comprises:

(b1) providing a spatial probe comprising a spatial tag to the spatial sample;

(b2) assessing the spatial tag in situ to obtain the spatial location of the spatial tag in the spatial sample; and

(b3) extending the recording tag by transferring information from the spatial tag in the spatial probe to the recording tag.

219. The method of embodiment 210, wherein step (b) comprises:

(b1) binding a molecular probe comprising a detectable label and a probe tag to the macromolecule or a moiety in proximity to the macromolecule in the spatial sample; and

(b2) assessing, e.g., observing, the detectable label to obtain spatial information of the molecular probe.

### VIII. EXAMPLES

The following examples are offered to illustrate but not to limit the methods, compositions, and uses provided herein.

### Example 1—Exemplary Assessment of Proteins in a Spatial Sample

This example describes an exemplary workflow for providing polypeptides in a tissue section with recording tags and other preparation steps for spatial analysis, including assessing spatial location of a plurality of proteins in the sample. Two exemplary methods for assessing spatial location in situ are described. Also described are exemplary procedures for binding molecular probes to the spatial sample and transferring information from the probe tag of the molecular probe to the recording tags.

### A1. Assessment of Spatial Location Using Barcoded Beads

One way of assessing the spatial location of the proteins in the sample is by providing the spatial sample with barcoded beads and decoding the barcoded beads in situ, as generally depicted in FIG. 2A-2F. Spatial tags are introduced into a mounted tissue section (fresh frozen or paraffin embedded) by overlaying and assembling DNA barcoded beads used as spatial probes on the surface of the mounted tissue section on the slide (Fischer et al., CSH Protoc (2008) pdb prot4991; Fischer et al., CSH Protoc (2008) pdb top36; Fischer et al., CSH Protoc. (2008) pdb.prot4988). Fresh-frozen tissue cryosections (10 μm thickness) are transferred onto the slide surface and undergo 4% formaldehyde fixation for about 20 minutes. The tissue section slides are dried with forced nitrogen air before the barcode bead overlay. Barcoded beads are brought into contact with the tissue section by incubating beads with the slides and spinning down the beads to form a monoloayer on the slide surface. The tissue surface is covered with beads attached non-specifically to the tissue surface through adhesive forces such as charge interactions, DNA hybridization, or reversible chemical coupling (FIG. 2B). In another embodiment, the beads are embedded in a hydrogel coated over the tissue section surface. In one embodiment, the beads are porous to accommodate a higher loading of barcodes on a bead (a porous 5 μm bead can be loaded with >10¹⁰ DNA barcodes, e.g. Daisogel SP-2000-5 porous silica beads). DNA barcodes (e.g., spatial tags) are attached to the bead via a photocleavable linker enabling easy removal and subsequent diffusive transfer of the barcodes to the tissue section. After decoding or sequencing the tissue-attached barcoded DNA beads (FIG. 2C), the DNA barcodes are released by enzymatic, chemical, or photocleavage of a cleavable linker. These barcodes permeate the tissue slice and anneal to the DNA stubs (e.g., recording tags) attached to proteins within the tissue slice (FIG. 2D). A polymerase extension step is used to write the barcodes to the DNA recording tags on the proteins, generating an extended recording tag. Further details are provided as follows:

Tissue Section Permeabilization

For fresh frozen samples, the tissue section permeabilized using standard methods such a 0.1%-1% TX-100 incubation prior to chemical activation of protein molecules (Fischer et al., CSH Protoc (2008) pdb prot4991; Fischer et al., CSH Protoc (2008) pdb top36; Fischer et al., CSH Protoc. (2008) pdb.prot4988). For FFPE tissue sections, the embedding media is removed (e.g. dewaxed in the case of paraffin), and the sections permeabilized using standard methods (Ramos-Vera et al., J Vet Diagn Invest. (2008) 20(4):393-413). Standard conditions for tissue permeabilization include incubation in 0.1%-1% TX-100 or NP-40 for 10-30 min. at 0.1 to 1%. Tween 20, Saponin, Digitonin can also be used at 0.2%-0.5% for 10-30 min (Fischer et al., CSH Protoc (2008) pdb top36). Acetone fixation is another method that generates tissue permeabilization.

Chemical Activation and DNA Tagging

After tissue section permeabilization and protein denaturation, in a preferred embodiment, proteins are chemically activated by incubation with an amine bifunctional bioconjugation reagent such as methyltetrazine-sulfo-NHS ester (Click Chemistry Tools); other bifunctional amine reactive bioconjugation reagents can also be employed (Hermanson, Bioconjugate Techniques, (2013) Academic Press). The density of DNA tagging can be controlled by titrating in non-activated amine modifying reagent such as mPEG-NHS ester. An exemplar activation condition includes incubating slides with 1 mM NHS-mTet for 30 min in PBS buffer (pH 7.4) to label epsilon-amine on lysines. Wash in 3× in PBS supplemented with 5 mM ethanolamine for 10 min. each to quench reaction. After activation and washing, a common DNA tag (comprising a suitable architecture for a recording tag) containing an iEDDA coupling label such as trans-cyclooctene (TCO), norbornene, or vinyl boronic acid is incubated with the tissue section to “click on” the DNA tags to the mTet moieties on the activated protein molecules (Knall et al., Tetrahedron Lett (2014) 55(34): 4763-4766). An exemplar coupling condition includes incubating the slide with 1 mM TCO-DNA stub for 1 hr in PBS buffer (pH 7.4).

DNA Barcoded Bead Distribution Over Tissue Section

In a preferred embodiment, DNA barcoded beads are generated through a split-pool synthesis strategy (Klein et al., Lab Chip (2017) 17(15): 2540-2541; Rodrigues et al., Science (2019) 363(6434):1463-1467). Each bead has a single population of DNA barcodes. In one embodiment, the beads are 0.5-10 um in diameter and contain a DNA barcode flanked by an upstream spacer sequence and a downstream primer extension sequence complementary to the DNA tag sequence attached to the proteins. In a preferred embodiment, the DNA barcodes are attached to the bead with a photo-cleavable linker, such as PC linker (PC Linker-CE Phosphoramidite, Glenn Research). In another embodiment, tissue section slides are assembled in a capillary gap flow-cell (˜50 um gap) such as the Te-Flow system from Tecan (Gunderson, Methods Mol Biol (2009) 529: 197-213). This provides a format for easily exchanging solutions on the slide surface.

In one embodiment, DNA barcoded beads are distributed across the surface of the tissue section, using the capillary gap flow cell system. The DNA barcode beads contain complementary sequences to the DNA tags on the proteins. This creates a “stickiness” of the barcoded beads to the surface of the tissue section with exposed DNA tags. In another embodiment, the beads are 0.5-10 um in diameter and contain both DNA barcodes and free amines on their surface. These free amine groups enhance adhesion to tissue surfaces since most tissues are slightly negatively charged (this is the mode to mount tissue slices on positively-charged slides for IHC). The barcoded beads can be covalently cross-linked to the tissue using standard fixation chemistry with glutaraldehyde.

Spatial Decoding of Barcoded Beads Assembled on Tissue Section

The assembled barcoded beads are spatially decoded in situ using fluorescent imaging and combinatorial hybridization-based approaches or in situ NGS sequencing (Gunderson et al., Genome Res (2004) 14(5): 870-877; Lee et al., Nat Protoc. (2015) 10(3): 442-458 Rodrigues et al., Science (2019) 363(6434): 1463-1467).

Transferring DNA Barcodes from Beads to DNA Tagged Proteins

After assembling barcode beads on the surface of the tissue section, the barcodes are photo-cleaved from the bead (via long wavelength UV exposure, e.g. 365 nm UV). A majority of linkages are cleaved, but not all, since photo-cleavage is generally only 70-90% efficient and can be adjusted by UV intensity and exposure time (3-100 mW/cm2 @ 340-365 nm for 1-60 min) (Bai et al., Proc Natl Acad Sci USA 100(2): 409-413). The cleaved barcodes diffuse into the tissue section and hybridize with their complement on DNA tags (e.g., recording tags) previously attached to proteins. After incubation for about 30 min., the tissue section is exposed to a polymerase extension mix to transfer barcode information from the hybridized barcode to the protein DNA recording tag.

### A2. Assessment of Spatial Location by Detecting Label of Molecular Probe

Another way to assess the spatial location of the proteins in the sample is performed by observing the detectable labels associated with molecular probes, as generally depicted in FIG. 1A-1D.

Proteins in the sample are first provided with DNA recording tags (FIG. 1A). A plurality of molecular probes are provided to the spatial sample, each molecular probe being associated with a detectable signal or label (e.g. fluorescence) which can be observed. Either before or after transferring information from the probe tag associated with the molecular probes to the recording tags (as described in section B of this example), an imaging step is performed to observe the detectable label (FIG. 1B). Multiple rounds of contacting the sample with molecular probes and observing the detectable labels can be performed. In some cases, one or more washes are performed after the signals are detected and before another cycle of molecular probes are provided. This assessment of the detectable label is performed for each set of molecular probes bound to the spatial sample. The position of each molecular probe observed is recorded and used in a later step to correlate to the probe tag information transferred to the recording tag. A known database or record of probe tag barcodes, molecular probe binding characteristics, and/or detectable labels associated with each molecular probe can be used.

B. Information Transfer from Probe Tag

Either after the spatial sample is labeled with spatial tags as described in section A1 of this example or as described in section A2 of this example, the spatial sample is contacted with multiple rounds of molecular probes, where each molecular probe is associated with a probe tag. The molecular probe binds to the proteins in the sample, and a reaction is carried out to extend the recording tag associated with the protein by transferring information from the probe tag of the molecular probe to the recording tag by extension. The transferring of information from the probe tag to the recording tag generates additional sequence on the recording tag (FIGS. 1C and 2E), generating an extended recording tag. The extended recording tags of the assay are released and/or amplified to be analyzed by next-generation sequencing (NGS) at this stage (FIGS. 1D and 2F). Alternatively, the proteins with the attached recording tags are released from the tissue and used in a further macromolecule analysis assay.

C. Harvesting of Proteins from Tissue Section

To use the proteins in a further analysis assay to obtain the sequence of the proteins (or a portion thereof), the tissue sections are scraped into a tube and standard trypsin digestion used to extract barcode labeled peptides. Trypsin digestion is accomplished by incubating slides in 0.1% trypsin in PBS for 12 hrs. at 37° C., and washed with three times with 1×PBS supplemented with 5 mM ethanolamine. In some cases, the peptide-DNA chimera can be directly ligated to sequencing beads and used in a further protein analysis assay (e.g., ProteoCode sequencing assay). The probe tag and optional spatial tag transferred as described is contained as a portion of the recording tag attached to peptides, which is suitable for use in a ProteoCode assay (see e.g., in International Patent Publication No. WO 2017/192633).

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

SEQUENCE TABLE
SEQUENCE TABLE

| SEQ | | |
|---|---|---|
| ID NO | Sequence (5′-3′) | Description |
| 1 | AATGATACGGCGACCACCGA | P5 primer |
| 2 | CAAGCAGAAGACGGCATACGAGAT | P7 primer |

1. A method of analyzing a macromolecule comprising:
(a) providing a spatial sample comprising a macromolecule associated with a recording tag at a spatial location;
(b) assessing the spatial location of the macromolecule in the spatial sample in situ;
(c1) binding a molecular probe comprising a probe tag to the macromolecule or a moiety in proximity to the macromolecule in the spatial sample;
(c2) extending the recording tag by transferring information from the probe tag in the molecular probe to the recording tag, wherein transferring information from the probe tag to the recording tag generates an extended recording tag;
(d) determining at least the sequence of the probe tag in the extended recording tag; and
(e) correlating the sequence of the probe tag determined in step (d) with the molecular probe and/or the spatial location assessed in step (b);
thereby associating information from the sequence of the extended recording tag or a portion thereof determined in step (d) with the spatial location assessed in step (b).

2. A method of analyzing a macromolecule comprising:
(a) providing a spatial sample comprising a macromolecule associated with a recording tag;
(b1) providing a spatial probe comprising a spatial tag to the spatial sample;
(b2) assessing the spatial tag in situ to obtain a spatial location of the spatial tag in the spatial sample;
(b3) extending the recording tag by transferring information from the spatial tag in the spatial probe to the recording tag;
(c1) binding a molecular probe comprising a probe tag to the macromolecule or a moiety in proximity to the macromolecule in the spatial sample;
(c2) extending the recording tag by transferring information from the probe tag in the molecular probe to the recording tag, wherein transferring information from the spatial tag and/or probe tag to the recording tag generates an extended recording tag;
(d) determining at least the sequence of the probe tag and spatial tag in the extended recording tag; and
(e) correlating the sequence of the probe tag determined in step (d) with the spatial tag assessed in step (b2);
thereby associating information from the sequence of the extended recording tag or a portion thereof, determined in step (d) with the spatial location of the spatial probe assessed in step (b2).

3. The method of claim 1, wherein assessing the spatial location of the macromolecule in the spatial sample in situ comprises:
(b1) providing a spatial probe comprising a spatial tag to the spatial sample;
(b2) assessing the spatial tag in situ to obtain a spatial location of the spatial tag in the spatial sample; and
(b3) extending the recording tag by transferring information from the spatial tag in the spatial probe to the recording tag;
and the method further comprises determining the sequence of the spatial tag in the extended recording tag at step (d).

4. The method of claim 1, wherein the macromolecule is a polypeptide.

5. The method of claim 1, wherein the molecular probe further comprises a detectable label, and the method further comprises assessing or observing the detectable label in order to assess the spatial location of the macromolecule in the spatial sample in situ at step (b).

6. (canceled)

7. The method of claim 1, further comprising repeating step (c1) and step (c2) sequentially two or more times.

8. (canceled)

9. The method of claim 3, wherein the spatial probe comprises a support and a spatial tag comprising a nucleic acid.

10-20. (canceled)

21. The method of claim 3, wherein the spatial tag comprises a sequence complementary to the recording tag or a portion thereof.

22. (canceled)

23. The method of claim 3, wherein the spatial probe associates with the spatial sample via charge interaction, DNA hybridization, and/or reversible chemical coupling.

24. The method of claim 3, wherein performing step (b2) comprises obtaining an image of the spatial sample or a portion thereof.

25-26. (canceled)

27. The method of claim 24, wherein performing step (b2) comprises using a microscope.

28. (canceled)

29. The method of claim 3, wherein the spatial tag is assessed in step (b2) using a decoder, wherein the decoder comprises a detectable label and a sequence complementary to the spatial tag or a portion thereof, wherein the detectable label comprises a radioisotope, a fluorescent label, a colorimetric label or an enzyme-substrate label.

30-33. (canceled)

34. The method of claim 3, wherein extending the recording tag by transferring information from the spatial tag to the recording tag comprises contacting the spatial sample with a polymerase and a nucleotide mix, thereby adding one or more nucleotides to the recording tag.

35-36. (canceled)

37. The method of claim 1, wherein the molecular probe comprises a targeting moiety capable of specific binding.

38-57. (canceled)

58. The method of claim 1, further comprising performing a macromolecule analysis assay for the macromolecule associated with the recording tag.

59-60. (canceled)

61. The method of claim 58, further comprising releasing the macromolecule associated with the recording tag from the spatial sample prior to performing the macromolecule analysis assay.

62. (canceled)

63. The method of claim 58, wherein the macromolecule is coupled directly or indirectly to a solid support prior to performing the macromolecule analysis assay.

64. The method of claim 58, wherein the macromolecule analysis assay comprises:
contacting the macromolecule with a binding agent capable of binding to the macromolecule, wherein the binding agent comprises a coding tag with identifying information regarding the binding agent; and
extending the recording tag associated with the macromolecule by transferring the information of the coding tag to the recording tag.

65. The method of claim 64, further comprising repeating one or more times:
contacting the macromolecule with an additional binding agent capable of binding to the macromolecule, wherein the additional binding agent comprises a coding tag with identifying information regarding the additional binding agent; and
extending the recording tag associated with the macromolecule by transferring the identifying information of the coding tag regarding the additional binding agent to the recording tag.

66-115. (canceled)

116. A method of analyzing a macromolecule comprising:
(a) providing a spatial sample comprising a macromolecule with a recording tag;
(b) binding a molecular probe comprising a detectable label and a probe tag to the macromolecule or a moiety in proximity to the macromolecule in the spatial sample;
(c) transferring information from the probe tag in the molecular probe to the recording tag to generate an extended recording tag;
(d) assessing or observing, the detectable label to obtain spatial information of the molecular probe;
(e) determining at least the sequence of the probe tag in the extended recording tag; and
correlating the sequence of the probe tag determined in step (e) with the molecular probe;
thereby associating information from the sequence determined in step (e) with its spatial information determined in step (d).

117-219. (canceled)


SEQUENCE LISTING (2 sequences)

SEQ ID NO: 1 - DNA, 20 bp, Artificial Sequence
  P5 primer
aatgatacggcgaccaccga

SEQ ID NO: 2 - DNA, 24 bp, Artificial Sequence
  P7 primer
caagcagaagacggcatacgagat